(19) United States
(12) Patent Application Publication
(10) Pub. No.: US 2021/0047297 A1
(43) Pub. Date: Feb. 18, 2021

(54) 4-(3-AMINO-6-FLUORO-1H-INDAZOL-5-YL)-1,2,6-TRIMETHYL-1,4-DIHYDROPYRIDINE-3,5-DIC ARBONITRILE COMPOUNDS FOR TREATING HYPERPROLIFERATIVE DISORDERS

(71) Applicant: Bayer Pharma Aktiengesellschaft, Berlin (DE)

(72) Inventors: Volker SCHULZE, Hohen Neuendorf (DE); Tobias HEINRICH, Berlin (DE); Clara CHRIST, Berlin (DE); Hans BRIEM, Berlin (US); Adelaide Clara FARIA ALVARES DE LEMOS, Berlin (DE); Benjamin BADER, Berlin (DE); Simon HOLTON, Berlin (DE); Ulf BÖMER, Glienicke (DE); Philip LIENAU, Berlin (DE); Lara Patricia KUHNKE, Berlin (DE)

(73) Assignee: Bayer Pharma Aktiengesellschaft, Berlin (DE)

(21) Appl. No.: 17/042,756

(22) PCT Filed: Mar. 25, 2019
(86) PCT No.: PCT/EP2019/057401

§ 371 Date: Sep. 28, 2020

(30) Foreign Application Priority Data
Mar. 28, 2018 (EP) 18164496.4

(51) Int. Cl. C07D 401/14; C07D 401/04

(52) U.S. Cl. CPC C07D 401/14; A61K 45/06; C07D 401/04

(57) ABSTRACT

The present invention covers 4-(3-amino-6-fluoro-1H-indazol-5-yl)-1,2,6-trimethyl-1,4-dihydropyridine-3,5-dicarbonitrile compounds of general formula (I): in which R¹, R² and R³ are as defined herein, methods of preparing said compounds, intermediate compounds useful for preparing said compounds, pharmaceutical compositions and combinations comprising said compounds and the use of said compounds for manufacturing pharmaceutical compositions for the treatment and/or prophylaxis of diseases.

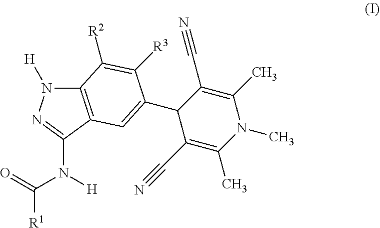

The invention relates to substituted 4-(3-amino-6-fluoro-1H-indazol-5-yl)-1,2,6-trimethyl-1,4-dihydropyridine-3,5-dicarbonitrile compounds, a process for their production and uses thereof.

### BACKGROUND OF THE INVENTION

The AMP-activated protein kinase (AMPK) is a sensor of the energy status in the cells, playing a key role in controlling their metabolism. Increases in the AMP and ADP cellular levels result in activation of AMPK leading to a general inhibition of anabolic pathways and activation of catabolic pathways that generate ATP. This leads to an increase in the ATP concentrations and restoration of the energy levels in the cells, thereby ensuring their energy homeostasis (Hardie et al, 2012. Nat Rev Mol Cell Biol, 13:251-62; Hardie and Alessi, 2013. BMC Biol, 11:36).

AMPK is a heterotrimeric complex with one catalytic (α) and two regulatory subunits (β and γ) at a 1:1:1 ratio. The catalytic subunit is a serine/threonine kinase and has two highly homologous isoforms (AMPKα1 and AMPKα2). AMPK activation requires phosphorylation of the activation loop (Thr 172) within the kinase domain of the α-catalytic subunit. CaMKKβ (calcium/calmodulin-dependent protein kinase kinase-β) and the tumor suppressor LKB1 are the best described kinases upstream of AMPK (Xiao et al, 2011. Nature, 472: 230-3).

For many years, AMPK was mainly perceived as a tumor suppressor in agreement with being a component of the LKB1 tumor suppressor cascade, which inhibits mTORC1. However, in the last few years, some studies suggested that AMPK might actually exert a pro-tumorigenic role in certain contexts (Faubert et al, 2015. Cancer Lett, 356:165-70; Jeon and Nay, 2015. Arch Pharm Res, 38:346-57). For instance, Liu and colleagues demonstrated that dysregulated MYC expression renders tumor cells sensitive to AMPK depletion. The authors showed that, due to their increased anabolism, MYC-dependent cells rely on AMPK to restore ATP levels and to prevent an energy crisis that results in apoptosis and cell death (Liu et al, 2012. Nature, 483:608-12). Another study has shown that AMPK plays a key role in maintaining NADPH levels under energy stress conditions such as nutrient deprivation as well as solid tumor formation in vivo. Under these circumstances, AMPK regulation of NADPH homeostasis promotes cancer cell survival (Jeon et al, 2012. Nature, 485:661-5). Consistently, the idea that AMPK is critical for tumor growth under nutrient and oxygen deprivation has been reported by other authors (Kato et al, 2002. Oncogene, 21:6082-90; Laderoute et al, 2006. Mol Cell Biol, 26:5336-47). Furthermore, AMPK mediates tumor survival during mitotic arrest (Doménech et al, 2015. Nat Cell Biol, 17:1304-16) and in response to ionizing radiation (Zannella et al, 2011. Radiother Oncol, 99:293-9). A growing list of reports suggests that the AR-CaMKKβ-AMPK axis plays a key role in prostate cancer survival (for review see Popovics et al, 2015. Expert Opin Ther Targets, 19:617-32). CaMKKβ is a transcriptional target of the androgen receptor (AR) and it is frequently overexpressed in prostate cancer. Of note, CaMKKβ-mediated activation of AMPK is required for androgen-dependent growth and migration of prostate cancer cells (Park et al, 2009. Mol Cancer Ther, 8:733-41; Frigo et al, 2011. Cancer Res, 71:528-37; Tennakoon et al, 2014. Oncogene, 33:5251-61). In this context, activated AMPK has been shown to induce autophagy and promote mitochondrial biogenesis, thereby promoting prostate cancer growth and survival (Shi et al, 2013. Mol Endocrinol, 27:280-95; Tennakoon et al, 2014. Oncogene, 33:5251-61).

Based on the increasing evidence supporting a pro-tumorigenic role of AMPK, inhibitors that can potently and selectively inhibit this protein could be useful for treating various tumor diseases.

WO2008/071451 A1 describes dihydropyridine derivatives having protein tyrosine kinase inhibitory activity and the use thereof for the treatment of c-Met-mediated diseases or c-Met-mediated conditions.

However, the state of the art does not describe the specific 4-(3-amino-6-fluoro-1H-indazol-5-yl)-1,2,6-trimethyl-1,4-dihydropyridine-3,5-dicarbonitrile compounds of general formula (I) of the present invention as described and defined herein.

It has now been found, and this constitutes the basis of the present invention, that the compounds of the present invention have surprising and advantageous properties.

In particular, the compounds of the present invention have surprisingly been found to effectively inhibit AMPK and may therefore be used for the treatment or prophylaxis of hyperproliferative disorders, such as cancer, for example.

### DESCRIPTION OF THE INVENTION

In accordance with a first aspect, the present invention covers compounds of general formula (I):

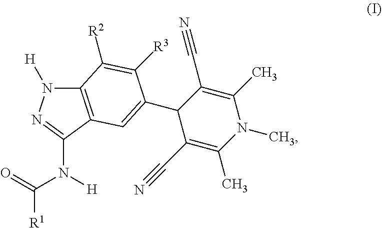

in which:
R¹ represents a group selected from

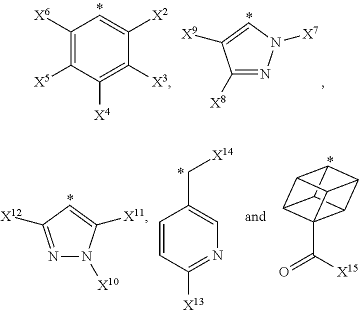

wherein “*” represents the point of attachment to the rest of the molecule,
and
wherein X² represents a hydrogen atom or a halogen atom or a group selected from C₁-C₂-alkyl and C₁-C₂-haloalkyl,
wherein X³ represents a hydrogen atom, wherein X⁴ represents a hydrogen atom or a (R⁴)(R⁵)N—(C₂-C₃-alkoxy)- group,
wherein X⁵ and X⁶, independently of each other, represent a hydrogen atom or a halogen atom,
wherein X⁷ represents a group selected from C₁-C₄-alkyl, C₂-C₄-hydroxyalkyl, methoxy-(C₂-C₄-alkyl)- and (R⁴)(R⁵)N—(C₂-C₃-alkoxy)-,
wherein X⁸ and X⁹, independently of each other, represent a hydrogen atom or a halogen atom or a group selected from methyl and C₁-haloalkyl,
wherein X¹⁰ represents a group selected from C₁-C₄-alkyl, C₂-C₄-hydroxyalkyl, methoxy-(C₂-C₄-alkyl)- and (R⁴)(R⁵)N—(C₂-C₃-alkoxy)-,
wherein X¹¹ and X¹², independently of each other, represent a hydrogen atom or a halogen atom or a group selected from methyl and C₁-haloalkyl,
wherein X¹³ and X¹⁴, independently of each other, represent a hydrogen atom or a methyl group,
and
wherein X¹⁵ represents a group selected from methoxy and —N(R⁴)(R⁵),
R² represents a halogen atom or a group selected from C₁-C₂-alkyl, C₁-C₂-fluoroalkyl and vinyl,
R³ represents a fluorine atom or a chlorine atom,
R⁴ and R⁵ represent, independently of each other, a hydrogen atom or a methyl group,
   or
R⁴ and R⁵ together with the nitrogen to which they are attached represent a nitrogen containing 4- to 6-membered heterocycloalkyl group,
   wherein said 4- to 6-membered nitrogen containing heterocycloalkyl group is optionally substituted, one or two times, with a methyl group,
      and stereoisomers, tautomers, N-oxides, hydrates, solvates, and salts thereof, and mixtures of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3 or 4, in particular 1, 2 or 3.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

Should a composite substituent be composed of more than one part, e.g. methoxy-(C₂-C₄-alkyl)-, it is possible for a given part to be attached at any suitable position of said composite substituent, e.g. it is possible for the methoxy part to be attached to any suitable carbon atom of the C₂-C₄-alkyl part of said methoxy-(C₂-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom.

The term “C₁-C₄-alkyl” means a linear or branched, saturated, monovalent hydrocarbon group having 1, 2, 3 or 4 carbon atoms, e.g. a methyl, ethyl, propyl, isopropyl, butyl, sec-butyl, isobutyl or tert-butyl. Particularly, said group has 2, 3 or 4 carbon atoms (“C₂-C₄-alkyl”), e.g. a ethyl, propyl, isopropyl, butyl, sec-butyl isobutyl, or tert-butyl group, or 1, 2 or 3 carbon atoms (“C₁-C₃-alkyl”), e.g. a methyl, ethyl, n-propyl or isopropyl group, more particularly 1 or 2 carbon atoms (“C₁-C₂-alkyl”), e.g. a methyl or ethyl group.

The term “C₂-C₄-hydroxyalkyl” means a linear or branched, saturated, monovalent hydrocarbon group in which the term “C₂-C₄-alkyl” is defined supra, and in which 1 hydrogen atom is replaced with a hydroxy group, e.g. a 2-hydroxyethyl, 3-hydroxypropyl, 2-hydroxypropyl, 1-hydroxypropan-2-yl, 3-hydroxy-2-methyl-propyl, 2-hydroxy-2-methyl-propyl, 2-hydroxybutyl, 3-hydroxybutyl, 4-hydroxybutyl group.

The term “C₁-C₂-haloalkyl” means a saturated, monovalent hydrocarbon group in which the term “C₁-C₂-alkyl” is as defined supra, and in which one or more of the hydrogen atoms are replaced, identically or differently, with a halogen atom. Particularly, said halogen atom is a fluorine atom. Said C₁-C₂-haloalkyl group is, for example, fluoromethyl, difluoromethyl, trifluoromethyl, 2-fluoroethyl, 2,2-difluoroethyl, 2,2,2-trifluoroethyl or pentafluoroethyl. Particularly, said group has 1 carbon atom (“C₁-haloalkyl”), e.g. a fluoromethyl, difluoromethyl or trifluoromethyl group.

The term “C₁-C₂-fluoroalkyl” means a saturated, monovalent hydrocarbon group in which the term “C₁-C₂-alkyl” is as defined supra, and in which one or more of the hydrogen atoms are replaced, identically or differently, with a fluorine atom. Said C₁-C₂-fluoroalkyl group is, for example, fluoromethyl, difluoromethyl, trifluoromethyl, 2-fluoroethyl, 2,2-difluoroethyl, 2,2,2-trifluoroethyl or pentafluoroethyl. Particularly, said group has 1 carbon atom (“C₁-fluoroalkyl”), e.g. a fluoromethyl, difluoromethyl or trifluoromethyl group.

The term “C₁-C₃-alkoxy” means a linear or branched, saturated, monovalent group of formula (C₁-C₃-alkyl)-O—, in which the term “C₁-C₃-alkyl” is as defined supra, e.g. a methoxy, ethoxy, n-propoxy or isopropoxy group.

The term nitrogen containing 4- to 6-membered heterocycloalkyl group means a monocyclic, saturated heterocycle with 4, 5 or 6 ring atoms in total, which contains one ring nitrogen atom and optionally one further ring heteroatom from the series N, O and S.

Said nitrogen containing 4- to 6-membered heterocycloalkyl group, without being limited thereto, can be a 4-membered ring, such as azetidinyl, for example; or a 5-membered ring, such as pyrrolidinyl, imidazolidinyl, pyrazolidinyl, 1,2-oxazolidinyl, 1,3-oxazolidinyl or 1,3-thiazolidinyl, for example; or a 6-membered ring, such as piperidinyl, morpholinyl, thiomorpholinyl, piperazinyl or 1,2-oxazinanyl, for example.

The term “C₁-C₄”, as used in the present text, e.g. in the context of the definition of “C₁-C₄-alkyl” means an alkyl group having a finite number of carbon atoms of 1 to 4, i.e. 1, 2, 3 or 4 carbon atoms.

Further, as used herein, the term “C₂-C₄”, as used in the present text, e.g. in the context of the definition of “C₂-C₄-hydroxyalkyl”, means a hydroxyalkyl group having a finite number of carbon atoms of 2 to 4, i.e. 2, 3 or 4 carbon atoms.

When a range of values is given, said range encompasses each value and sub-range within said range.

For example:

“C₁-C₄” encompasses C₁, C₂, C₃, C₄, C₁-C₄, C₁-C₃, C₁-C₂, C₂-C₄, C₂-C₃, and C₃-C₄;

“C₂-C₄” encompasses C₂, C₃, C₄, C₂-C₄, C₂-C₃, and C₃-C₄,

“C₂-C₃” encompasses C₂, C₃ and C₂-C₃.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as 2H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a direct route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

In another embodiment the present invention concerns a deuterium-containing compound of general formula (I) having 1, 2, 3 or 4 deuterium atoms, particularly with 1, 2 or 3 deuterium atoms.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of the present invention of the structural formula (I) optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres.

Preferred isomers are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the mixtures according to conventional processes, for example, by the formation of diastereoisomeric salts using an optically active acid or base or formation of covalent diastereomers. The optically active bases or acids are then liberated from the separated diastereomeric salts. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Deicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, the compounds of the present invention contain a indazole moiety and can exist as a 1H tautomer, or a 2H tautomer, or even a mixture in any amount of the two tautomers, namely:

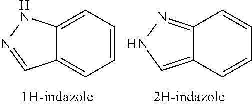

The present invention includes all possible tautomers of the compounds of the present invention as single tautomers, or as any mixture of said tautomers, in any ratio.

Further, the compounds of the present invention can exist as N-oxides, which are defined in that at least one nitrogen atom of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, dimethylsulfoxide, tetrahydrofuran, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with, unless defined, unknown stoichiometric composition.

As used herein, the term “in vivo hydrolysable ester” means an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkyloxy-carbonyloxy-C₁-C₆ alkyl esters, e.g. 1-cyclohexyloxycarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, it being possible for said esters to be formed at any carboxy group in the compounds of the present invention.

An in vivo hydrolysable ester of a compound of the present invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted alkanoyl, benzoyl and phenylacetyl, alkoxycarbonyl (to give alkyl carbonate esters), N,N-dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with a second embodiment of the first aspect, the present invention covers compounds of general formula (I), supra, in which:
R¹ represents a group selected from

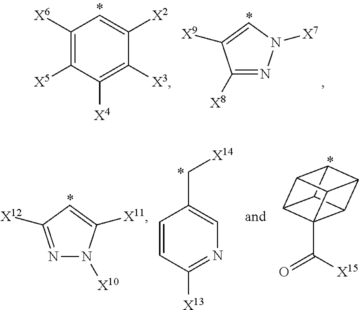

wherein “*” represents the point of attachment to the rest of the molecule,
and
wherein X² represents a hydrogen atom or a fluorine atom or a chlorine atom or a group selected from ethyl and trifluoromethyl,
wherein X³ represents a hydrogen atom,
wherein X⁴ represents a hydrogen atom or a 2-pyrrolidin-1-ylethoxy- group,
wherein X⁵ and X⁶, independently of each other, represent a hydrogen atom or a fluorine atom,
wherein X⁷ represents a group selected from methyl, ethyl and isopropyl,
wherein X⁸ and X⁹, independently of each other, represent a hydrogen atom or a chlorine atom or a methyl group,
wherein X¹⁰ represents a group selected from methyl, ethyl, 2-hydroxy-2-methylpropyl, 2-methoxyethyl and 2-pyrrolidin-1-ylethoxy,
wherein X¹¹ and X¹², independently of each other, represent a hydrogen atom or a group selected from methyl and trifluoromethyl,
wherein X¹³ and X¹⁴, independently of each other, represent a hydrogen atom or a methyl group,
and
wherein X¹⁵ represents a group selected from methoxy, azetidin-1-yl and 4-methylpiperazin-1-yl,
R² represents an iodine atom or a group selected from methyl, ethyl, trifluoromethyl and vinyl,
R³ represents a fluorine atom,
   and stereoisomers, tautomers, N-oxides, hydrates, solvates, and salts thereof, and mixtures of same.

In accordance with a third embodiment of the first aspect, the present invention covers compounds of general formula (I), supra, in which:
R¹ represents a group selected from

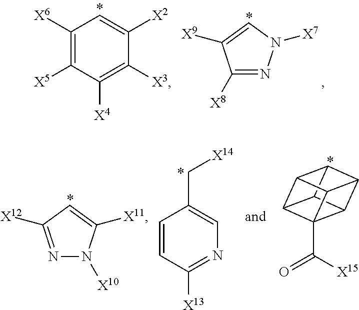

wherein “*” represents the point of attachment to the rest of the molecule,
and
wherein X² represents a hydrogen atom or a fluorine atom or a chlorine atom or a group selected from ethyl and trifluoromethyl,
wherein X³ represents a hydrogen atom,
wherein X⁴ represents a hydrogen atom or a 2-pyrrolidin-1-ylethoxy- group,
wherein X⁵ and X⁶, independently of each other, represent a hydrogen atom or a fluorine atom,
wherein X⁷ represents a group selected from methyl, ethyl and isopropyl,
wherein X⁸ represents a hydrogen atom or a methyl group,
wherein X⁹ represent a hydrogen atom or a chlorine atom,
wherein X¹⁰ represents a group selected from methyl, ethyl, 2-hydroxy-2-methylpropyl, 2-methoxyethyl and 2-pyrrolidin-1-ylethoxy,
wherein X¹¹ represents a hydrogen atom or trifluoromethyl group,
wherein X¹² represent a hydrogen atom or a group selected from methyl and trifluoromethyl,
wherein X¹³ and X¹⁴, independently of each other, represent a hydrogen atom or a methyl group,
and
wherein X¹⁵ represents a group selected from methoxy, azetidin-1-yl and 4-methylpiperazin-1-yl,
R² represents an iodine atom or a group selected from methyl, ethyl, trifluoromethyl and vinyl,
R³ represents a fluorine atom,
   and stereoisomers, tautomers, N-oxides, hydrates, solvates, and salts thereof, and mixtures of same.

In accordance with a fourth embodiment of the first aspect, the present invention covers compounds of general formula (I), supra, which are selected from the group consisting of:
N-[5-(3,5-dicyano-1,2,6-trimethyl-1,4-dihydropyridin-4-yl)-6-fluoro-7-iodo-1H-indazol-3-yl]-1-ethyl-3-methyl-1H-pyrazole-5-carboxamide,
2-chloro-N-[5-(3,5-dicyano-1,2,6-trimethyl-1,4-dihydropyridin-4-yl)-6-fluoro-7-iodo-1H-indazol-3-yl]benzamide,
N-[5-(3,5-dicyano-1,2,6-trimethyl-1,4-dihydropyridin-4-yl)-6-fluoro-7-iodo-1H-indazol-3-yl]-1-methyl-3-(trifluoromethyl)-1H-pyrazole-4-carboxamide,
2-chloro-N-[5-(3,5-dicyano-1,2,6-trimethyl-1,4-dihydropyridin-4-yl)-6-fluoro-7-iodo-1H-indazol-3-yl]-4-[2-(pyrrolidin-1-yl)ethoxy]benzamide,
N-[5-(3,5-dicyano-1,2,6-trimethyl-1,4-dihydropyridin-4-yl)-6-fluoro-7-methyl-1H-indazol-3-yl]-1-ethyl-3-methyl-1H-pyrazole-5-carboxamide,
2-chloro-N-[5-(3,5-dicyano-1,2,6-trimethyl-1,4-dihydropyridin-4-yl)-6-fluoro-7-methyl-1H-indazol-3-yl]benzam ide,
N-[5-(3,5-dicyano-1,2,6-trimethyl-1,4-dihydropyridin-4-yl)-6-fluoro-7-methyl-1H-indazol-3-yl]-2,6-difluorobenzamide,
N-[5-(3,5-dicyano-1,2,6-trimethyl-1,4-dihydropyridin-4-yl)-6-fluoro-7-methyl-1H-indazol-3-yl]-2-ethylbenzamide,
N-[5-(3,5-dicyano-1,2,6-trimethyl-1,4-dihydropyridin-4-yl)-6-fluoro-7-methyl-1H-indazol-3-yl]-1-methyl-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-5-fluoro-2-(trifluoromethyl)benzamide,
2-chloro-N-[5-(3,5-dicyano-1,2,6-trimethyl-1,4-dihydropyridin-4-yl)-6-fluoro-7-methyl-1H-indazol-3-yl]-6-fluorobenzamide,
N-[5-(3,5-dicyano-1,2,6-trimethyl-1,4-dihydropyridin-4-yl)-6-fluoro-7-methyl-1H-indazol-3-yl]-1-ethyl-3-methyl-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-4-[2-(pyrrolidin-1-yl)ethoxy]benzamide,
N-[5-(3,5-dicyano-1,2,6-trimethyl-1,4-dihydropyridin-4-yl)-6-fluoro-7-methyl-1H-indazol-3-yl]-2-(6-methylpyridin-3-yl)acetamide,
N-[5-(3,5-dicyano-1,2,6-trimethyl-1,4-dihydropyridin-4-yl)-6-fluoro-7-methyl-1H-indazol-3-yl]-1-(2-methoxyethyl)-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-[2-(pyrrolidin-1-yl)ethyl]-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-(2-hydroxy-2-methylpropyl)-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-(2-hydroxy-2-methylpropyl)-5-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-[2-(pyrrolidin-1-yl)ethyl]-5-(trifluoromethyl)-1H-pyrazole-4-carboxamide,
2-chloro-N-[5-(3,5-dicyano-1,2,6-trimethyl-1,4-dihydropyridin-4-yl)-6-fluoro-7-methyl-1H-indazol-3-yl]-4-[2-(pyrrolidin-1-yl)ethoxy]benzamide,
(2R)—N-[5-(3,5-dicyano-1,2,6-trimethyl-1,4-dihydropyridin-4-yl)-6-fluoro-7-methyl-1H-indazol-3-yl]-2-(pyridin-3-yl)propanamide,
methyl 4-{[5-(3,5-dicyano-1,2,6-trimethyl-1,4-dihydropyridin-4-yl)-6-fluoro-7-methyl-1H-indazol-3-yl]carbamoyl}cubane-1-carboxylate,
4-(azetidin-1-ylcarbonyl)-N-[5-(3,5-dicyano-1,2,6-trimethyl-1,4-dihydropyridin-4-yl)-6-fluoro-7-methyl-1H-indazol-3-yl]cubane-1-carboxamide,
N-[5-(3,5-dicyano-1,2,6-trimethyl-1,4-dihydropyridin-4-yl)-6-fluoro-7-methyl-1H-indazol-3-yl]-4-[(4-methylpiperazin-1-yl)carbonyl]cubane-1-carboxamide,
N-[5-(3,5-dicyano-1,2,6-trimethyl-1,4-dihydropyridin-4-yl)-7-ethyl-6-fluoro-1H-indazol-3-yl]-2,6-difluorobenzamide,
N-[5-(3,5-dicyano-1,2,6-trimethyl-1,4-dihydropyridin-4-yl)-7-ethyl-6-fluoro-1H-indazol-3-yl]-2-ethylbenzamide,
N-[5-(3,5-dicyano-1,2,6-trimethyl-1,4-dihydropyridin-4-yl)-7-ethyl-6-fluoro-1H-indazol-3-yl]-1-(propan-2-yl)-1H-pyrazole-5-carboxamide,
4-chloro-N-[5-(3,5-dicyano-1,2,6-trimethyl-1,4-dihydropyridin-4-yl)-7-ethyl-6-fluoro-1H-indazol-3-yl]-1-methyl-1H-pyrazole-5-carboxamide,
N-[5-(3,5-dicyano-1,2,6-trimethyl-1,4-dihydropyridin-4-yl)-7-ethyl-6-fluoro-1H-indazol-3-yl]-1-ethyl-3-methyl-1H-pyrazole-5-carboxamide,
2-chloro-N-[5-(3,5-dicyano-1,2,6-trimethyl-1,4-dihydropyridin-4-yl)-7-ethyl-6-fluoro-1H-indazol-3-yl]benzamide,
2-chloro-N-[5-(3,5-dicyano-1,2,6-trimethyl-1,4-dihydropyridin-4-yl)-6-fluoro-7-(trifluoromethyl)-1H-indazol-3-yl]benzamide,
N-[5-(3,5-dicyano-1,2,6-trimethyl-1,4-dihydropyridin-4-yl)-6-fluoro-7-(trifluoromethyl)-1H-indazol-3-yl]-1-ethyl-3-methyl-1H-pyrazole-5-carboxamide, and
N-[5-(3,5-dicyano-1,2,6-trimethyl-1,4-dihydropyridin-4-yl)-7-ethenyl-6-fluoro-1H-indazol-3-yl]-1-ethyl-3-methyl-1H-pyrazole-5-carboxamide,
   and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from

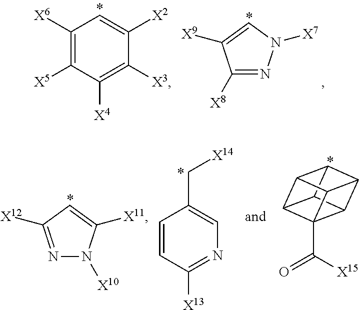

wherein “*” represents the point of attachment to the rest of the molecule,
and
wherein X² represents a hydrogen atom or a halogen atom or a group selected from C₁-C₂-alkyl and C₁-C₂-haloalkyl,
wherein X³ represents a hydrogen atom,
wherein X⁴ represents a hydrogen atom or a (R⁴)(R⁵)N—(C₂-C₃-alkoxy)- group,
wherein X⁵ and X⁶, independently of each other, represent a hydrogen atom or a halogen atom,
wherein X⁷ represents a group selected from C₁-C₄-alkyl, C₂-C₄-hydroxyalkyl, methoxy-(C₂-C₄-alkyl)- and (R⁴)(R⁵)N—(C₂-C₃-alkoxy)-,
wherein X⁸ and X⁹, independently of each other, represent a hydrogen atom or a halogen atom or a group selected from methyl and C₁-haloalkyl,
wherein X¹⁰ represents a group selected from C₁-C₄-alkyl, C₂-C₄-hydroxyalkyl, methoxy-(C₂-C₄-alkyl)- and (R⁴)(R⁵)N—(C₂-C₃-alkoxy)-,
wherein X¹¹ and X¹², independently of each other, represent a hydrogen atom or a halogen atom or a group selected from methyl and C₁-haloalkyl,
wherein X¹³ and X¹⁴, independently of each other, represent a hydrogen atom or a methyl group,
and
wherein X¹⁵ represents a group selected from methoxy and —N(R⁴)(R⁵),
   and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from

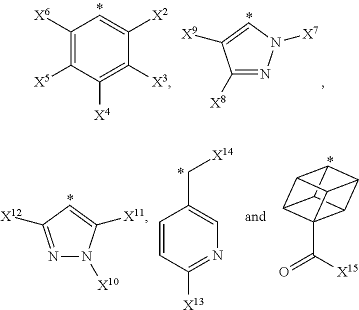

wherein “*” represents the point of attachment to the rest of the molecule,
and
wherein X² represents a hydrogen atom or a fluorine atom or a chlorine atom or a group selected from ethyl and trifluoromethyl,
wherein X³ represents a hydrogen atom,
wherein X⁴ represents a hydrogen atom or a 2-pyrrolidin-1-ylethoxy- group,
wherein X⁵ and X⁶, independently of each other, represent a hydrogen atom or a fluorine atom,
wherein X⁷ represents a group selected from methyl, ethyl and isopropyl,
wherein X⁸ and X⁹, independently of each other, represent a hydrogen atom or a chlorine atom or a methyl group,
wherein X¹⁰ represents a group selected from methyl, ethyl, 2-hydroxy-2-methylpropyl, 2-methoxyethyl and 2-pyrrolidin-1-ylethoxy,
wherein X¹¹ and X¹², independently of each other, represent a hydrogen atom or a group selected from methyl and trifluoromethyl,
wherein X¹³ and X¹⁴, independently of each other, represent a hydrogen atom or a methyl group,
and
wherein X¹⁵ represents a group selected from methoxy, azetidin-1-yl and 4-methylpiperazin-1-yl,
   and stereoisomers, tautomers, N-oxides, hydrates, solvates, and salts thereof, and mixtures of same.

In a further embodiment of the first aspect, the present invention covers compounds of formula (I), supra, in which:
R¹ represents a group selected from

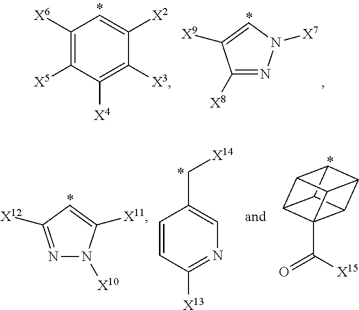

wherein “*” represents the point of attachment to the rest of the molecule,
and
wherein X² represents a hydrogen atom or a fluorine atom or a chlorine atom or a group selected from ethyl and trifluoromethyl,
wherein X³ represents a hydrogen atom,
wherein X⁴ represents a hydrogen atom or a 2-pyrrolidin-1-ylethoxy- group,
wherein X⁵ and X⁶, independently of each other, represent a hydrogen atom or a fluorine atom,
wherein X⁷ represents a group selected from methyl, ethyl and isopropyl,
wherein X⁸ represents a hydrogen atom or a methyl group,
wherein X⁹ represent a hydrogen atom or a chlorine atom,
wherein X¹⁰ represents a group selected from methyl, ethyl, 2-hydroxy-2-methylpropyl, 2-methoxyethyl and 2-pyrrolidin-1-ylethoxy,
wherein X¹¹ represents a hydrogen atom or trifluoromethyl group,
wherein X¹² represent a hydrogen atom or a group selected from methyl and trifluoromethyl,
wherein X¹³ and X¹⁴, independently of each other, represent a hydrogen atom or a methyl group,
and
wherein X¹⁵ represents a group selected from methoxy, azetidin-1-yl and 4-methylpiperazin-1-yl,
   and stereoisomers, tautomers, N-oxides, hydrates, solvates, and salts thereof, and mixtures of same.

In a further embodiment of the first aspect, the present invention covers compounds of formula (I), supra, in which:
R² represents a halogen atom or a group selected from C₁-C₂-alkyl, C₁-C₂-fluoroalkyl and vinyl,
   and stereoisomers, tautomers, N-oxides, hydrates, solvates, and salts thereof, and mixtures of same.

In a further embodiment of the first aspect, the present invention covers compounds of formula (I), supra, in which:
R² represents an iodine atom or a group selected from methyl, ethyl, trifluoromethyl and vinyl,
   and stereoisomers, tautomers, N-oxides, hydrates, solvates, and salts thereof, and mixtures of same.

In a further embodiment of the first aspect, the present invention covers compounds of formula (I), supra, in which:
R³ represents a fluorine atom or a chlorine atom,
   and stereoisomers, tautomers, N-oxides, hydrates, solvates, and salts thereof, and mixtures of same.

In a further embodiment of the first aspect, the present invention covers compounds of formula (I), supra, in which:
R³ represents a fluorine atom,
   and stereoisomers, tautomers, N-oxides, hydrates, solvates, and salts thereof, and mixtures of same.

In a further embodiment of the first aspect, the present invention covers compounds of formula (I), supra, in which:
R⁴ and R⁵ represent, independently of each other, a hydrogen atom or a methyl group,
or
R⁴ and R⁵ together with the nitrogen to which they are attached represent a nitrogen containing 4- to 6-membered heterocycloalkyl group,
   wherein said 4- to 6-membered nitrogen containing heterocycloalkyl group is optionally substituted, one or two times, with a methyl group,
      and stereoisomers, tautomers, N-oxides, hydrates, solvates, and salts thereof, and mixtures of same.

In a further embodiment of the first aspect, the present invention covers compounds of formula (I), supra, in which:
R⁴ and R⁵ represent, independently of each other, a hydrogen atom or a methyl group,
   and stereoisomers, tautomers, N-oxides, hydrates, solvates, and salts thereof, and mixtures of same.

In a further embodiment of the first aspect, the present invention covers compounds of formula (I), supra, in which:
R⁴ and R⁵ together with the nitrogen to which they are attached represent a nitrogen containing 4- to 6-membered heterocycloalkyl group,
   wherein said 4- to 6-membered nitrogen containing heterocycloalkyl group is optionally substituted, one or two times, with a methyl group,
      and stereoisomers, tautomers, N-oxides, hydrates, solvates, and salts thereof, and mixtures of same.

General Procedures

The compounds according to the invention can be prepared according to the following Schemes 1 through 4.

The Schemes and procedures described below illustrate synthetic routes to the compounds of general formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents R¹, R², R³, A, X², X³, X⁴, X⁵, X⁶, X⁷, X⁸, X⁹, X¹⁰, X¹², X¹³, X¹⁴ and X¹⁵ can be achieved before and/or after the exemplified transformations. These modifications can be such as the introduction of protecting groups, cleavage of protecting groups, reduction or oxidation of functional groups, dehydrogenation, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art.

Specific examples are described in the subsequent paragraphs.

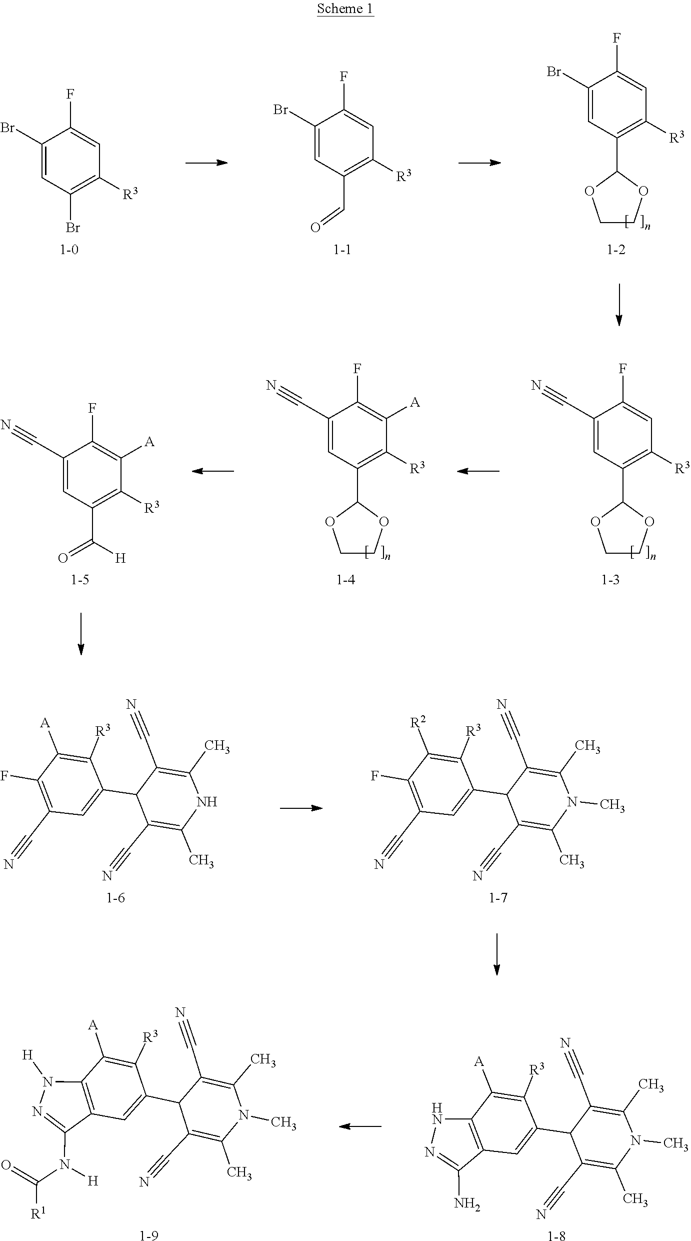

Scheme 1: Route for the preparation of compounds of general formula (I) for R²=A represented by formula (1-9), wherein R′ and R³ have the meaning as given for general formula (I), supra and A represents a halogen or an alkyl group that can optionally be substituted with one or more fluorine atoms and n represents 1 or 2.

Reagents of general formula (1-0) are commercially available and can be reacted with a suitable alkyl lithium reagent like for example n-butyl lithium or sec-butyl lithium or tert-butyl lithium, preferably n-butyl lithium in a suitable solvent, like an ether, preferably diethyl ether at low temperature, preferably between −60° C. and −78° C. to form an aryl lithium reagent that can then be reacted with N,N-dimethylformamide at a temperature between −78° C. and r.t. to furnish compounds of general formula (1-1).

Intermediates of general formula (1-1) can be converted to intermediates of general formula (1-2) by reaction with ethane-1,2-diol or propane-1,3-diol, preferably ethane-1,2-diol in a suitable solvent system, such as, for example, toluene or chloroform at a temperature between room temperature and the boiling point of the respective solvents with a catalytic amount of an acid like for example 4-methylbenzenesulfonic acid or camphersulphonic acid. Preferably the reaction is carried out at the boiling point of the respective solvents, whereby the water formed in the reaction can be removed from the reaction by methods known to those skilled in the art, such as, for example, azeotropic removal of water (Dean-Stark conditions) or with molecular sieves, to furnish intermediates of general formula (1-2). A detailed description for the formation of acetals as protecting groups is found, for example, in T. W. Greene, Protective Groups in Organic Synthesis, John Wiley & Sons, 1999, 3rd Ed., or in P. Kocienski, Protecting Groups, Thieme Medical Publishers, 2000.

Intermediates of general formula (1-2) can be converted to intermediates of general formula (1-3) by reaction with a cyanide salt, like zink cyanide or copper cyanide, preferably copper cyanide, in a suitable solvent system, such as, for example, dimethylacetamide or N-methylpyrrolidinone, preferably N-methylpyrrolidinone, at a temperature between 100° C. and 200° C., preferably 175° C. Preferably the reaction can be carried out in a microwave oven to furnish intermediates of general formula (1-3).

Intermediates of general formula (1-3) can be reacted with a suitable alkyl lithium reagent like for example n-butyl lithium or with a suitable lithium amide base like for example lithium 2,2,6,6-tetramethylpiperidin-1-ide, in a suitable solvent, like an ether or a cyclic ether, preferably tetrahydrofuran at low temperature, preferably between −60° C. and −78° C. to form an aryl lithium reagent that can then be reacted with an electrophile, like iodine or 2-iodo-1H-isoindole-1,3(2H)-dione (NIS), or 2-bromo-1H-isoindole-1,3(2H)-dione (NBS) or 2-chloro-1H-isoindole-1,3(2H)-dione (NCS) or 1,2-dibromo-1,1,2,2-tetrafluoroethane or an alkyl halide or alkyl triflate, preferably an alkyl iodide like for example methyl iodide or ethyl iodide or an alkyl iodide or alkyl triflate substituted with one or more fluorine atoms like for example 1,1,1-triflouro-2-iodoethane or 2,2,2-trifluoroethyl methanesulfonate at a temperature between −78° C. and r.t. to furnish compounds of general formula (1-4).

Intermediates of general formula (1-4) can be converted to intermediates of general formula (1-5) by reaction with an excess of an aqueous solution of an acid, such as for example hydrochloric acid or sulfuric acid in a suitable solvent system, such as, for example, 1,4-dioxane or acetone at a temperature between room temperature and the boiling point of the respective solvents. A detailed description for the cleavage of acetal protecting groups as needed for the conversion of intermediates of general formula (1-4) to intermediates of general formula (1-5) is found, for example, in T. W. Greene, Protective Groups in Organic Synthesis, John Wiley & Sons, 1999, 3rd Ed., or in P. Kocienski, Protecting Groups, Thieme Medical Publishers, 2000.

Intermediates of general formula (1-5) can be reacted with an excess of (2E)-3-aminobut-2-enenitrile, preferably 2 equivalents, in a suitable solvent, like for example acetic acid or a mixture of acetic acid and a further inert solvent like for example N-methylpyrrolidinone, at a temperature between 60° C. and 120° C., preferably 90° C. to furnish compounds of general formula (1-6).

Intermediates of general formula (1-6) can be reacted with a suitable base like for example potassium carbonate or cesium carbonate or sodium hydride in a suitable solvent, like for example N,N-dimethylformamide or dimethylacetamide or N-methylpyrrolidinone or an ether or a cyclic ether, preferably N,N-dimethylformamide or tetrahydrofuran at a temperature between 0° C. and 50° C., preferably at room temperature to furnish compounds of general formula (1-7).

Intermediates of general formula (1-7) can be reacted with an hydrazine or hydrazine hydrate, preferably with an excess of hydrazine hydrate, in a suitable solvent, like for example an aliphatic alcohol, preferably 2-propanol or 1-propanol or 1-butanol at a temperature between 80° C. and 150° C., preferably in 1-propanol or 1-butanol at the boiling point of the respective solvent or a temperature above the boiling point of the respective solvent using a microwave oven or a sealed microwave vial preferably at 100° C. or 120° C. or 130° C., to furnish compounds of general formula (1-8).

Intermediates of general formula (1-8) can be subjected to a peptide coupling by reaction with a carboxylic acid of formula R¹—C(═O)OH, in which R¹ is as defined for compounds of general formula (I), in the presence of a peptide coupling reagent, selected from HATU (O-(7-azabenzotriazol-1-yl)-N,N,N′,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or T3P (2,4,6-tripropyl-1,3,5,2,4,6-trioxatriphosphinane 2,4,6-trioxide), all of them being well known to the person skilled in the art and all of them being commercially available, in the presence of a base such as a tertiary aliphatic amine of the formula N(C₁-C₄-alkyl)₃, or sodium bicarbonate, or potassium carbonarte, in an appropriate solvent such as N,N-dimethylformamide, N,N-dimethylacetamide, dimethylsulfoxide, tetrahydrofuran, dichloromethane or N-methyl pyrrolidin-2-one in a temperature range from 0° C. to the boiling point of the respective solvent to furnish compounds of general formula (1-9).

Alternatively, intermediates of general formula (1-8) can be reacted with an acylating reagent, like an acid fluoride, acid chloride or acid bromide, or an acid anhydride of the general formula

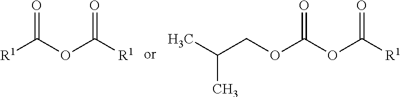

preferably in the presence of a base such as a tertiary aliphatic amine of the formula N(C₁-C₄-alkyl)₃, or pyridine or sodium bicarbonate, or potassium carbonarte optionally in the presence of a catalytic amount of N,N-dimethylpyridin-4-amine, in an appropriate solvent such as for example tetrahydrofuran, dichloromethane or N-methyl pyrrolidin-2-one in a temperature range from 0° C. to the boiling point of the respective solvent, preferably at room temperature to furnish compounds of general formula (1-9). Specific examples are described in the Experimental Section.

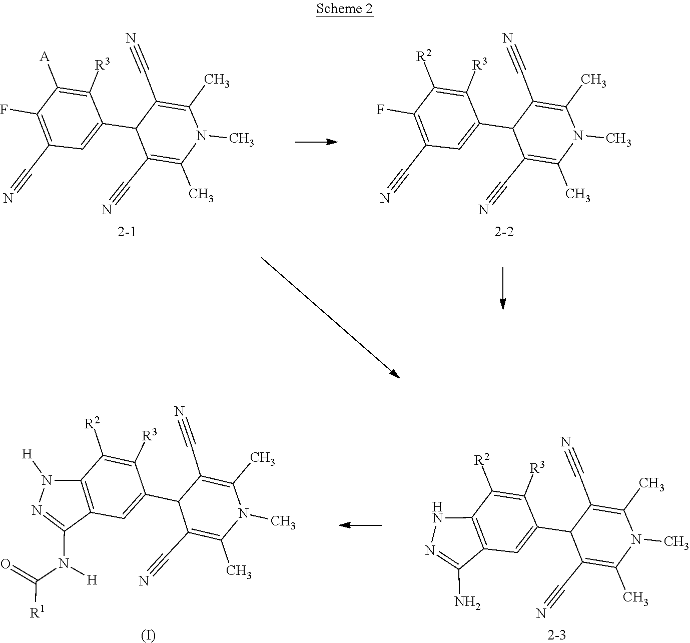

Scheme 2: Route for the preparation of compounds of general formula (I), wherein R¹, R² and R³ have the meaning as given for general formula (I), supra and A represents a halogen or an alkyl group that can optionally be substituted with one or more fluorine atoms.

Intermediates of general formula (2-1) in which A represents an idodine atom can be converted to intermediates of general formula (2-2) in which A represents a trifluoromethyl group by reaction with methyl 2,2-difluoro-2-(fluorosulfonyl)acetate in the presence of copper iodide, in a suitable solvent system, such as, for example, N,N-dimethylformamide, dimethylacetamide or

N-methylpyrrolidinone, or mixtures of these solvents preferably mixtures of N,N-dimethylformamide and N-methylpyrrolidinone, at a temperature between 75° C. and 150° C., preferably 100° C. to furnish such intermediates of general formula (2-2). Alternatively, intermediates of general formula (2-1) in which A represents an idodine atom can be converted to intermediates of general formula (2-2) in which A represents a vinyl group or an alkyl group or an alkyl group that is substituted with one or more fluorine atoms, by reaction with a boronic acid, a potassium trifluoroborate(1-) derivative or a boronic acid derivative, like for example potassium trifluoro(methyl)borate(1-), 2,4,4,5,5-pentamethyl-1,3,2-dioxaborolane, potassium ethyl(trifluoro)borate(1-), potassium ethenyl(trifluoro)borate(1-), triethenylboroxin, pyridine-triethenylboroxin, ethenylboronic acid, 2-ethenyl-4,4,5,5-tetramethyl-1,3,2-dioxaborolane, 2-ethenyl-6-methyl-1,3,6,2-dioxazaborocane-4,8-dione, 4,4,5,5-tetramethyl-2-(trifluoromethyl)-1,3,2-dioxaborolane, potassium trifluoro(trifluoromethyl)borate(1-), potassium trifluoro(2,2,2-trifluoroethyl)borate(1-) or potassium trifluoro(pentafluoroethyl)borate(1-) in the presence of a suitable palladium catalyst like for example dichloro[1,1′-bis(diphenyllphosphino)-ferrocene]palladium(II)×dichloromethane or dichlorobis(triphenylphosphine)palladium(II) or dichlorobis(triphenylphosphine)palladium(II) with additional triphenyl phosphine or tetrakis(triphenylphosphine)palladium and a suitable base like for example sodium bicarbonate, sodium carbonate or potassium carbonate, potassium phosphate or cesium carbonate or aqueous solutions of these bases in a suitable solvent or solvent mixture like ethanol, 1-propanol, 2-propanol, tetrahydrofuran, 1,2-dimethoxyethane, N,N-dimethylformamide, dimethylacetamide, N-methylpyrrolidinone or mixtures of any of these solvents together with water at temperatures between 60° C. and 150° C. preferably at 100° C. or at the boiling point of the respective solvent or solvent mixture. Such reactions of intermediates of general formula (2-1) in which A represents an idodine atom to intermediates of general formula (2-2) can also be carried out in a microwave oven.

Intermediates of general formula (2-1) and in the same way also intermediates of general formula (2-2) can be reacted with an hydrazine or hydrazine hydrate, preferably with an excess of hydrazine hydrate, in a suitable solvent, like for example an aliphatic alcohol, preferably 2-propanol or 1-propanol or 1-butanol at a temperature between 80° C. and 150° C., preferably in 1-propanol or 1-butanol at the boiling point of the respective solvent or a temperature above the boiling point of the respective solvent using a microwave oven or a sealed microwave vial preferably at 100° C. or 120° C. or 130° C., to furnish compounds of general formula (2-3).

Intermediates of general formula (2-3) can be subjected to a peptide coupling by reaction with a carboxylic acid of formula R¹—C(═O)OH, in which R¹ is as defined for compounds of general formula (I), in the presence of a peptide coupling reagent, selected from HATU (O-(7-azabenzotriazol-1-yl)-N,N,N′,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or T3P (2,4,6-tripropyl-1,3,5,2,4,6-trioxatriphosphinane 2,4,6-trioxide), all of them being well known to the person skilled in the art and all of them being commercially available, in the presence of a base such as a tertiary aliphatic amine of the formula N(C₁-C₄-alkyl)₃, or sodium bicarbonate, or potassium carbonarte, in an appropriate solvent such as N,N-dimethylformamide, N,N-dimethylacetamide, dimethylsulfoxide, tetrahydrofuran, dichloromethane or N-methyl pyrrolidin-2-one in a temperature range from 0° C. to the boiling point of the respective solvent to furnish compounds of general formula (I).

Alternatively, intermediates of general formula (2-3) can be reacted with an acylating reagent, like an acid fluoride, acid chloride or acid bromide, or an acid anhydride of the general formula

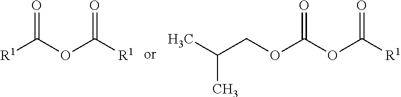

preferably in the presence of a base such as a tertiary aliphatic amine of the formula N(C₁-C₄-alkyl)₃, or pyridine or sodium bicarbonate, or potassium carbonarte optionally in the presence of a catalytic amount of N,N-dimethylpyridin-4-amine, in an appropriate solvent such as for example tetrahydrofuran, dichloromethane or N-methyl pyrrolidin-2-one in a temperature range from 0° C. to the boiling point of the respective solvent, preferably at room temperature to furnish compounds of general formula (I). Specific examples are described in the Experimental Section.

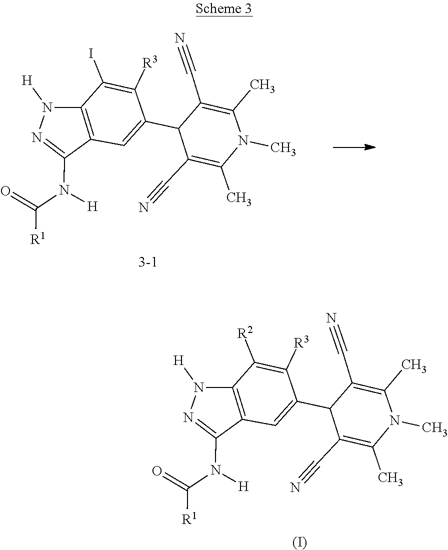

Scheme 3: Route for the preparation of compounds of general formula (I), wherein R¹, R² and R³ have the meaning as given for general formula (I).

Intermediates of general formula (3-1) can be converted compounds of general formula (I) in which R² represents a trifluoromethyl group by reaction with methyl 2,2-difluoro-2-(fluorosulfonyl)acetate in the presence of copper iodide, in a suitable solvent system, such as, for example, N,N-dimethylformamide, dimethylacetamide or N-methylpyrrolidinone, or mixtures of these solvents preferably mixtures of N,N-dimethylformamide and N-methylpyrrolidinone, at a temperature between 75° C. and 150° C., preferably 100° C. to furnish compounds of general formula (I) in which R² represents a trifluoromethyl group. Alternatively, intermediates of general formula (3-1) can be converted to compounds of general formula (I) in which R² represents a vinyl group or an alkyl group or an alkyl group that is substituted with one or more fluorine atoms, by reaction with a boronic acid, a potassium trifluoroborate(1-) derivative or a boronic acid derivative, like for example potassium trifluoro(methyl)borate(1-), 2,4,4,5,5-pentamethyl-1,3,2-dioxaborolane, potassium ethyl(trifluoro)borate(1-), potassium ethenyl(trifluoro)borate(1-), triethenylboroxin, pyridine-triethenylboroxin, ethenylboronic acid, 2-ethenyl-4,4,5,5-tetramethyl-1,3,2-dioxaborolane, 2-ethenyl-6-methyl-1,3,6,2-dioxazaborocane-4,8-dione, 4,4,5,5-tetramethyl-2-(trifluoromethyl)-1,3,2-dioxaborolane, potassium trifluoro(trifluoromethyl)borate(1-), potassium trifluoro(2,2,2-trifluoroethyl)borate(1-) or potassium trifluoro(pentafluoroethyl)borate(1-) in the presence of a suitable palladium catalyst like for example dichloro[1,1′-bis(diphenylphosphino)ferrocene]palladium(II)×dichloromethane or dichlorobis(triphenylphosphine)palladium(II) or dichlorobis(triphenylphosphine)palladium(II) with additional triphenyl phosphine or tetrakis(triphenylphosphine)palladium and a suitable base like for example sodium bicarbonate, sodium carbonate or potassium carbonate, potassium phosphate or cesium carbonate or aqueous solutions of these bases in a suitable solvent or solvent mixture like ethanol, 1-propanol, 2-propanol, tetrahydrofuran, 1,2-dimethoxyethane, N,N-dimethylformamide, dimethylacetamide, N-methylpyrrolidinone or mixtures of any of these solvents together with water at temperatures between 60° C. and 150° C. preferably at 100° C. or at the boiling point of the respective solvent or solvent mixture. Such reactions of intermediates of general formula (3-1) to icompounds of general formula (I) can also be carried out in a microwave oven.

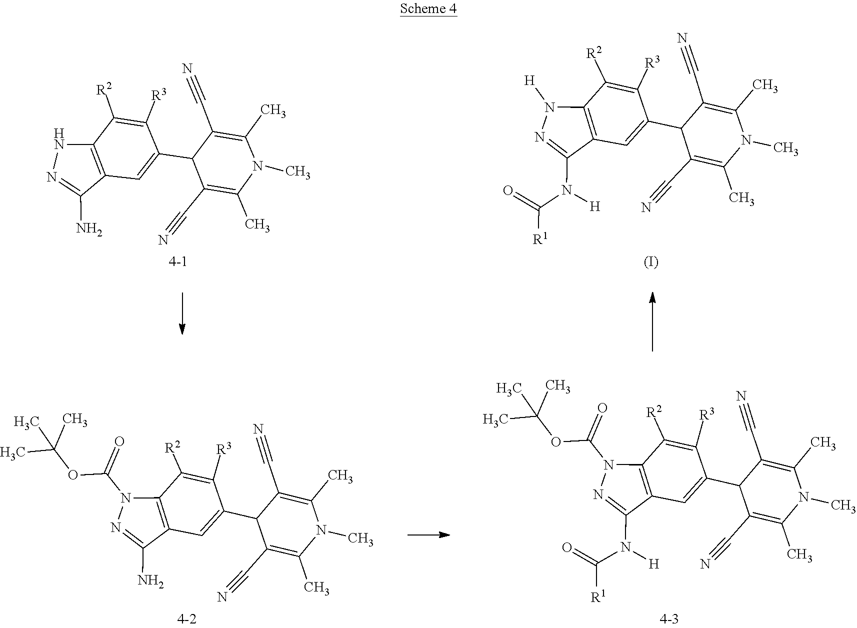

Scheme 4: Route for the preparation of compounds of general formula (I), wherein R¹, R² and R³ have the meaning as given for general formula (I).

Intermediates of general formula (4-1) can be reacted with a suitable base like for example or pyridine, triethylamine or diisopropylethylamine and a catalytic amount of N,N-dimethylpyridin-4-amine with di-tert-butyl dicarbonate in a suitable solvent, like for example N,N-dimethylformamide or dimethylacetamide or N-methylpyrrolidinone or an ether or a cyclic ether like for example tetrahydrofuran, preferably N,N-dimethylformamide or a mixture of N,N-dimethylformamide and tetrahydrofuran at a temperature between 0° C. and 50° C., preferably at room temperature to furnish compounds of general formula (4-2). A detailed overview for the formation Boc protected amino groups is found, for example, in T. W. Greene, Protective Groups in Organic Synthesis, John Wiley & Sons, 1999, 3rd Ed., or in P. Kocienski, Protecting Groups, Thieme Medical Publishers, 2000.

Intermediates of general formula (4-2) can be subjected to a peptide coupling by reaction with a carboxylic acid of formula R¹—C(═O)OH, in which R¹ is as defined for compounds of general formula (I), in the presence of a peptide coupling reagent, selected from HATU (O-(7-azabenzotriazol-1-yl)-N,N,N′,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or T3P (2,4,6-tripropyl-1,3,5,2,4,6-trioxatriphosphinane 2,4,6-trioxide), all of them being well known to the person skilled in the art and all of them being commercially available, in the presence of a base such as a tertiary aliphatic amine of the formula N(C₁-C₄-alkyl)₃, or sodium bicarbonate, or potassium carbonarte, in an appropriate solvent such as N,N-dimethylformamide, N,N-dimethylacetamide, dimethylsulfoxide, tetrahydrofuran, dichloromethane or N-methyl pyrrolidin-2-one in a temperature range from 0° C. to the boiling point of the respective solvent to furnish compounds of general formula (4-3).

Alternatively, intermediates of general formula (4-2) can be reacted with an acylating reagent, like an acid fluoride, acid chloride or acid bromide, or an acid anhydride of the general formula

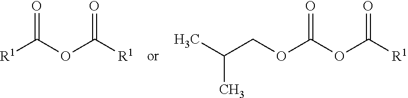

preferably in the presence of a base such as a tertiary aliphatic amine of the formula N(C₁-C₄-alkyl)₃, or pyridine or sodium bicarbonate, or potassium carbonarte optionally in the presence of a catalytic amount of N,N-dimethylpyridin-4-amine, in an appropriate solvent such as for example tetrahydrofuran, dichloromethane or N-methyl pyrrolidin-2-one in a temperature range from 0° C. to the boiling point of the respective solvent, preferably at room temperature to furnish compounds of general formula (4-3).

Intermediates of general formula (4-3) can be converted to compounds of general formula (I) by reaction with an excess of an aqueous solution of an acid, such as for example hydrochloric acid or sulfuric acid in a suitable solvent system, such as, for example, dichloromethane, tetrahydrofuran or 1,4-dioxane at a temperature between 0° C. and the boiling point of the respective solvents, preferably room temperature. A detailed overview for the cleavage of Boc protection groups as needed for the conversion of intermediates of general formula (4-3) to compounds of general formula (I) is found, for example, in T. W. Greene, Protective Groups in Organic Synthesis, John Wiley & Sons, 1999, 3rd Ed., or in P. Kocienski, Protecting Groups, Thieme Medical Publishers, 2000.

It is known to the person skilled in the art that, if there are a number of reactive centers on a starting or intermediate compound, it may be necessary to block one or more reactive centers temporarily by protective groups in order to allow a reaction to proceed specifically at the desired reaction center. A detailed description for the use of a large number of proven protective groups is found, for example, in T. W. Greene, Protective Groups in Organic Synthesis, John Wiley & Sons, 1999, 3rd Ed., or in P. Kocienski, Protecting Groups, Thieme Medical Publishers, 2000.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC may be applied. The compounds of the present invention which possess a sufficiently basic or acidic functionality, may result as a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of the compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, free acid, solvate, inclusion complex) of a compound of the present invention as isolated and described herein is not necessarily the only form in which said compound can be applied to a biological assay in order to quantify the specific biological activity.

Salts of the compounds of formula (I) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the example section.

Pure diastereomers and pure enantiomers of the compounds and salts according to the invention can be obtained e.g. by asymmetric synthesis, by using chiral starting compounds in synthesis or by splitting up enantiomeric and diasteriomeric mixtures obtained in synthesis.

Enantiomeric and diastereomeric mixtures can be split up into the pure enantiomers and pure diastereomers by methods known to the person skilled in the art. Preferably, diastereomeric mixtures are separated by crystallization, in particular fractional crystallization, or chromatography. Enantiomeric mixtures can be separated e.g. by forming diastereomers with a chiral auxiliary agent, resolving the diastereomers obtained and removing the chiral auxiliary agent. As chiral auxiliary agents, for example, chiral acids can be used to separate enantiomeric bases such as e.g. mandelic acid and chiral bases can be used to separate enantiomeric acids by formation of diastereomeric salts. Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxiliary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

Another aspect of the invention is the process for the preparation of the compounds of claims 1 to 4 according to the examples as well as the intermediates used for their preparation.

The intermediates used for the synthesis of the compounds of claims of formula (I) as described herein, as well as their use for the synthesis of the compounds of formula (I), are one further aspect of the present invention. Preferred intermediates are the Intermediate Examples as disclosed herein.

The present invention covers the intermediate compounds which are disclosed in the Experimental Section of this text, infra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action, which could not have been predicted. Compounds of the present invention have surprisingly been found to effectively inhibit the activity of AMPK and it is possible therefore that said compounds be used for the treatment or prophylaxis of diseases, preferably hyperproliferative disorders in humans and animals.

Compounds of the present invention can be utilized to inhibit the activity of AMPK. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

In another aspect, the present invention provides methods of treating cancer, which cancer is selected from breast cancer, cancer of the respiratory tract, brain cancer, prostate cancer, testicular cancer, endometrial cancer, cervical cancer, ovarian cancer, vaginal cancer, vulvar cancer, sarcoma of the uterus, anal cancer, colon cancer, colorectal cancer, oesophageal cancer, gallbladder cancer, gastric cancer, pancreatic cancer, rectal cancer, small-intestine cancer, salivary gland cancer, bladder cancer, penile cancer, kidney cancer, renal pelvis cancer, ureter cancer, urethral cancer, human papillary renal cancer, eye cancer, liver cancer, skin cancer, head-and-neck cancer, lymphoma, sarcoma and leukemia.

In another aspect, the present invention provides the use of a compound of general formula (I) of the present invention, or a pharmaceutically acceptable salt, polymorph, metabolite, hydrate, solvate or ester thereof, for the treatment of cancer, which cancer is selected from breast cancer, cancer of the respiratory tract, brain cancer, prostate cancer, testicular cancer, endometrial cancer, cervical cancer, ovarian cancer, vaginal cancer, vulvar cancer, sarcoma of the uterus, anal cancer, colon cancer, colorectal cancer, oesophageal cancer, gallbladder cancer, gastric cancer, pancreatic cancer, rectal cancer, small-intestine cancer, salivary gland cancer, bladder cancer, penile cancer, kidney cancer, renal pelvis cancer, ureter cancer, urethral cancer, human papillary renal cancer, eye cancer, liver cancer, skin cancer, head-and-neck cancer, lymphoma, sarcoma and leukemia.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

In the context of the present invention, the term “treating” or “treatment” means combatting, inhibiting, delaying, hindering, alleviating, diminishing, limiting, reducing, suppressing, repressing or curing of a disease, of a complaint, of an illness, of an injury or of a health disorder, or of the development, the course or the progression of same.

In the context of the present invention, the term “prevention” or “prophylaxis” means avoiding or decreasing of the risk of getting, suffering from, sustaining or having a disease, a complaint, an illness, an injury or health disorder, or the development, the course, the progression or the symptoms of same.

Said treatment and/or prevention of a disease, a complaint, an illness, an injury or health disorder can be carried out partially or totally.

The compounds of the present invention can be used in particular in therapy and prevention, i.e. prophylaxis, of hyperproliferative disorders, more particularly cancer.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyperproliferative disorders, particularly benign hyperproliferative disorders, more particularly cancer.

The pharmacological activity of the compounds according to the invention can be explained by their ability to inhibit the activity of AMPK.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment and/or prophylaxis of diseases, in particular hyperproliferative disorders, particularly cancer.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment and/or prophylaxis of diseases, in particular hyperproliferative disorders, particularly cancer disorders.

In accordance with a further aspect, the present invention covers the use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer disorders.

In accordance with a further aspect, the present invention covers a method of treatment or prophylaxis of diseases, in particular hyperproliferative disorders, particularly cancer, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular medicaments, comprising compounds of general formula (I), as described supra, or stereoisomers, tautomers, N-oxides, hydrates, solvates, salts thereof, particularly pharmaceutically acceptable salts, or mixtures of same, and one or more excipient(s), in particular one or more pharmaceutically acceptable excipient(s).

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®),
flow regulators, lubricants, glidants and mould release agents (for example magnesium stearate, stearic acid, talc, highly-disperse silicas (such as, for example, Aerosil®),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and block copolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to pharmaceutical compositions which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a hyperproliferative disorder, particularly cancer.

Particularly, the present invention covers a pharmaceutical combination, which comprises:
one or more first active ingredients, in particular compounds of general formula (I) as defined supra, and
one or more further active ingredients, in particular anti-cancer agents.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-cancer agents.

Examples of Anti-Cancer Agents Include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporf in, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the present invention can be administered as the sole pharmaceutical agent or in combination with one or more medical therapeutic means (e.g. surgical intervention, irradiation) and/or medical devices or appliances (e.g. breathing apparatuses, pacemaker implants, electrostimulation, stents).

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Therapeutically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg body weight. The average daily rectal dosage regimen will preferably be from 0.01 to 200 mg/kg body weight. The average daily vaginal dosage regimen will preferably be from 0.01 to 200 mg/kg body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg body weight.

Nevertheless, it may be necessary to deviate from the stated amounts, depending on the body weight, the route of administration, the individual behavior towards the active substance, the type of preparation and the time or interval at which the application takes place. Thus, in some cases, it may be sufficient to get by with less than the aforementioned minimum quantity, while in other cases the above-mentioned upper limit must be exceeded. In the case of the application of larger quantities, it may be advisable to distribute these in several doses throughout the day.

Nevertheless, it may be necessary to deviate from the stated amounts, depending on the body weight, the route of administration, the individual behavior towards the active substance, the type of formulation and the time or interval at which the application takes place. Thus, in some cases, it may be possible to achieve the desired effect with less than the aforementioned minimum quantity, while in other cases the above-mentioned upper limit must be exceeded. In the case of the application of larger quantities, it may be advisable to distribute these in several doses throughout the day.

### EXPERIMENTAL SECTION

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ(intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “Minimum Height”<1%.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

| Abbreviation | Meaning |
|---|---|
| AcOH | acetic acid (ethanoic acid) |
| aq. | aqueous |
| Boc | t-butoxycarbonyl |
| BOP | (benzotriazol-1-yloxy)tris(dimethylamino) |
| | phosphonium hexafluorophosphate |
| br | broad |
| Brett-Phos | 2-(dicyclohexylphosphino)-3,6-dimethoxy-2′-4′- |
| | 6′-tri-i-propyl-1,1′-biphenyl |
| Cl | chemical ionisation |
| Cs₂CO₃ | caesium carbonate |
| d | doublet |
| dd | doublet of doublets |
| DAD | diode array detector |
| DBU | 1,8-diazabicyclo(5.4.0)undec-7-ene |
| DCC | N,N′-dicyclohexylcarbodiimide |
| DCM | dichloromethane |
| dd | double-doublet |
| DIC | N,N′-diisopropylcarbodiimide |
| DIPEA | diisopropylethylamine |
| DMA | dimethylacetamide |
| DMAP | N,N-dimethylpyridin-4-amine |
| DMF | N,N-dimethylformamide |
| DMSO | dimethyl sulfoxide |
| dppf | 1,1′-bis(diphenylphosphino)ferrocene |
| dt | double-triplet |
| EDC, EDCI | 1-ethyl-3-(3-dimethylaminopropyl)carbodiimide |
| ELSD | Evaporative Light Scattering Detector |
| EtOAc | ethyl acetate |
| EtOH | ethanol |
| eq. | equivalent |
| ESI | electrospray (ES) ionisation |
| h | hour |
| HATU | 1-[bis(dimethylamino)methylene]-1H-1,2,3- |
| | triazolo[4,5-b]-pyridinium 3-oxid |
| | hexafluorophosphate |
| HBTU | (o-benzotriazole-10yl)-N,N,N′,N,- |
| | tetramethyluronium hexafluorophosphate |
| HCl | hydrochloric acid |
| HPLC | high performance liquid chromatography |
| K₂CO₃ | potassium carbonate |
| LC-MS | liquid chromatography mass spectrometry |
| m | multiplet |
| min | minute |
| MeCN | acetonitrile |
| MeOH | methanol |
| MS | mass spectrometry |
| MW | molecular weight |
| NaCl | sodium chloride |
| NaHCO₃ | sodium hydrogen carbonate or sodium |
| | bicarbonate |
| NBS | 2-bromo-1H-isoindole-1,3(2H)-dione |
| NCS | 2-chloro-1H-isoindole-1,3(2H)-dione |
| NIS | 2-iodo-1H-isoindole-1,3(2H)-dione |
| NMP | N-methylpyrrolidinone |
| NMR | nuclear magnetic resonance spectroscopy: |
| | chemical shifts (δ) are given in ppm. The |
| | chemical shifts were corrected by setting |
| | the DMSO signal to 2.50 ppm unless otherwise |
| | stated. |
| PDA | Photo Diode Array |
| Pd/C | palladium on activated charcoal |
| PdCl₂(PPh₃)₂ | dichlorobis(triphenylphosphine)palladium(II) |
| Pd(dba)₂ | bis-(dibenzylideneacetone)palladium(0) complex |
| Pd₂(dba)₃ | tris-(dibenzylideneacetone)dipalladium(0) |
| | chloroform complex |
| Pd(dppf)Cl₂ | dichloro[1,1′-bis(diphenylphosphino)ferrocene] |
| | palladium(II) |
| Pd(dppf)Cl₂•CH₂Cl₂ | dichloro[1,1′-bis(diphenylphosphino)ferrocene] |
| | palladium(II) dichloromethane adduct |
| Pd-Brett-Phos-pre-cat | chloro[2-(dicyclohexylphosphino)-3,6- |
| | dimethoxy-2′-4′-6′-tri-iso-propyl-1,1′-biphenyl] |
| | [2-(2-aminoethyl)phenyl]palladium(II) |
| Pd-tBu-X-Phos-pre-cat | chloro(2-di-tert-butylphosphino-2′,4′,6-tri- |
| | isopropyl-1,1′-biphenyl)[2-(2-aminoethyl) |
| | phenyl] palladium(II) |
| Pd-X-Phos-pre-cat | chloro(2-dicyclohexylphosphino-2′,4′,6′-tri- |
| | isopropyl-1,1′-biphenyl)[2-(2-aminoethyl) |
| | phenyl] palladium(II) methyl-tert-butylether |
| | adduct |
| PPh₃ | triphenylphosphine |
| PyBOP | (benzotriazol-1-yloxy)tripyrrolidinophosphonium |
| | hexafluorophosphate |
| q | quartet |
| r.t. or rt or RT | room temperature |
| Rt | retention time (as measured either with HPLC or |
| | UPLC) in minutes |
| s | singlet |
| S-Phos | dicyclohexyl(2′,6′-dimethoxybiphenyl-2-yl) |
| | phosphine |
| sat. | saturated |
| SIBX | stabilized 2-iodoxybenzoic acid |
| SM | starting material |
| SQD | Single-Quadrupole-Detector |
| T3P | propylphosphonic anhydride |
| tBu-X-Phos | 2-di-tert-butylphosphino-2′,4′,6′-tri-isopropyl- |
| | 1,1′-biphenyl |
| t | triplet |
| td | triple-doublet |
| TEA | triethylamine |
| TFA | trifluoroacetic acid |
| THF | tetrahydrofuran |
| UPLC | ultra performance liquid chromatography |

Other abbreviations have their meanings customary per se to the skilled person. The various aspects of the invention described in this application are illustrated by the following examples which are not meant to limit the invention in any way.

Specific Experimental Descriptions

NMR peak forms in the following specific experimental descriptions are stated as they appear in the spectra, possible higher order effects have not been considered.

Reactions employing microwave irradiation may be run with a Biotage Initator® microwave oven optionally equipped with a robotic unit. The reported reaction times employing microwave heating are intended to be understood as fixed reaction times after reaching the indicated reaction temperature.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example pre-packed silica gel cartridges, e.g. from Separtis such as Isolute® Flash silica gel or Isolute® Flash NH₂ silica gel in combination with a Isolera® autopurifier (Biotage) and eluents such as gradients of e.g. hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The percentage yields reported in the following examples are based on the starting component that was used in the lowest molar amount. Air and moisture sensitive liquids and solutions were transferred via syringe or cannula and introduced into reaction vessels through rubber septa. Commercial grade reagents and solvents were used without further purification. The term “concentrated in vacuo” refers to the use of a Buchi rotary evaporator at a minimum pressure of approximately 15 mm of Hg. All temperatures are reported uncorrected in degrees Celsius (C).

In order that this invention may be better understood, the following examples are set forth. These examples are for the purpose of illustration only, and are not to be construed as limiting the scope of the invention in any manner. All publications mentioned herein are incorporated by reference in their entirety.

Analytical Conditions

UPLC-MS Standard Procedures

UPLC-MS-data given in the subsequent specific experimental descriptions refer (unless otherwise noted) to the following conditions:

Method 1:

| | |
|---|---|
| System: | Waters Acquity UPLC-MS: Binary Solvent Manager, |
| | Sample Manager/Organizer, Column Manager, PDA, |
| | ELSD, SQD 3001 |
| Column: | Acquity BEH C18 1.7 50 × 2.1 mm |
| Solvent: | A = water + 0.1% vol. formic acid (99%) |
| | B = acetonitrile |
| Gradient: | 0-1.6 min 1-99% B, 1.6-2.0 min 99% B |
| Flow: | 0.8 ml/min |
| Temperature: | 60° C. |
| Injection: | 2.0 μl |
| Detection: | DAD scan range 210-400 nm |
| | MS ESI+, ESI−, scan range 160-1000 m/z |
| | ELSD |

Method 2:

| | |
|---|---|
| System: | Waters Acquity UPLC-MS: Binary Solvent Manager, |
| | Sample Manager/Organizer, Column Manager, PDA, |
| | ELSD, SQD 3001 |
| Column: | Acquity BEH C18 1.7 50 × 2.1 mm |
| Solvent: | A = water + 0.2% vol. ammonia (32%) |
| | B = acetonitrile |
| Gradient: | 0-1.6 min 1-99% B, 1.6-2.0 min 99% B |
| Flow: | 0.8 ml/min |
| Temperature: | 60° C. |
| Injection: | 2.0 μl |
| Detection: | DAD scan range 210-400 nm |
| | MS ESI+, ESI−, scan range 160-1000 m/z |
| | ELSD |

Preparative HPLC Conditions

“Purification by preparative HPLC” in the subsequent experimental descriptions refers to the following conditions (unless otherwise noted):

Preparative HPLC (Method Acidic):

| | |
|---|---|
| System: | Waters Autopurificationsystem: Pump 2545, Sample |
| | Manager 2767, CFO, DAD 2996, ELSD 2424, SQD |
| | 3001 |
| Column: | XBridge C18 5 μm 100 × 30 mm |
| Solvent: | A = water + 0.1% vol. formic acid (99%) |
| | B = acetonitrile |
| Gradient: | 0-1 min 1% B, 1-8 min 1-99% B, 8-10 min 99% B |
| Flow: | 50 ml/min |
| Temperature: | RT |
| Solution: | max. 250 mg/2.5 ml dimethyl sufoxide or DMF |
| Injection: | 1 × 2.5 ml |
| Detection: | DAD scan range 210-400 nm |
| | MS ESI+, ESI−, scan range 160-1000 m/z |

Preparative HPLC (Method Basic):

| | |
|---|---|
| System: | Waters Autopurificationsystem: Pump 2545, Sample |
| | Manager 2767, CFO, DAD 2996, ELSD 2424, |
| | SQD 3001 |
| Column: | XBridge C18 5 μm 100 × 30 mm |
| Solvent: | A = water + 0.2% vol. ammonia (32%) |
| | B = acetonitrile |
| Gradient: | 0-1 min 1% B, 1-8 min 1-99% B, 8-10 min 99% B |
| Flow: | 50 ml/min |
| Temperature: | RT |
| Solution: | max. 250 mg/2.5 ml dimethyl sufoxide or DMF |
| Injection: | 1 × 2.5 ml |
| Detection: | DAD scan range 210-400 nm |
| | MS ESI+, ESI−, scan range 160-1000 m/z |

Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

Chemical names were generated using the ACD/Name software from ACD/Labs. In some cases generally accepted names of commercially available reagents were used in place of ACD/Name generated names.

Optical rotations were measured using a JASCO P2000 Polarimeter. Typical, a solution of the compound with a concentration of 1 mg/mL to 15 mg/mL was used for the measurement. The specific rotation [α]_{D} was calculated according to the following formula:

$$[ α ] D = \frac{∝}{β \times d}$$

In this equation, α is the measured rotation in degrees; d is the path length in decimeters and β is the concentration in g/mL.

### EXPERIMENTAL SECTION—INTERMEDIATES

### Intermediate 1

### 5-bromo-2,4-difluorobenzaldehyde

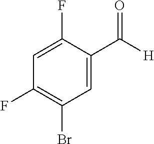 [SMILES: O=Cc1cc(Br)c(F)cc1F]

To a stirred solution of 1,5-dibromo-2,4-difluorobenzene (20.0 g, 73.6 mmol) in diethyl ether (400 mL) in a dry ice bath was added n-butyl lithium in hexanes (31 ml, 2.5 M, 77 mmol) so that the temperature of the reaction mixture stayed between −65° C. and −70° C. and then the mixture was stirred at −70° C. for further 0.5 h. N, N-dimethylformamide (7.1 ml, 92 mmol) was slowly added so that the temperature of the reaction mixture stayed between −65° C. and −70° C. and then the mixture was stirred at −70° C. for further 0.5 h. The dry ice bath was removed and the mixture was allowed to warm up to 0° C. A mixture of water (180 mL) and acetic acid (11 ml, 200 mmol) was added and the mixture was stirred for 15 min. The phases were separated and the aqueous phase was extracted with ethyl acetate. The organic phases were combined and washed with saturated sodium chloride solution, dried (sodium sulfate), filtered and the solvent was removed in vacuum. Silicagel chromatography gave 12.6 g (77% yield) of the title compound.

LC-MS (Method 1): Rₜ=1.09 min; MS (ESIpos): m/z=220 [M+H]⁺

¹H-NMR (400 MHz, DMSO-d6) δ[ppm]: 2.518 (0.45), 7.716 (4.55), 7.738 (4.98), 7.742 (4.92), 7.765 (4.71), 8.141 (4.86), 8.159 (6.75), 8.179 (4.87), 10.079 (16.00).

### Intermediate 2

### 2-(5-bromo-2,4-difluorophenyl)-1,3-dioxolane

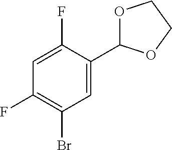 [SMILES: Fc1cc(F)c(C2OCCO2)cc1Br]

To a stirred solution of 5-bromo-2,4-difluorobenzaldehyde (12.0 g, 54.3 mmol) in chloroform (860 ml) were added ethane-1,2-diol (4.6 ml, 81 mmol) and 4-methylbenzenesulfonic acid monohydrate (1.03 g, 5.43 mmol) and the mixture was stirred at reflux using an inverse Dean-Stark trapp for 16 h. An aqueous solution of sodium bicarbonate was added, and the mixture was extracted with dichloromethane. The organic phase was washed with saturated sodium chloride solution, dried (sodium sulfate), filtered and the solvent was removed in vacuum. Silicagel chromatography gave 12.2 g (84% yield) of the title compound.

LC-MS (Method 1): Rₜ=1.18 min; MS (ESIpos): m/z=265 [M+H]⁺

¹H-NMR (400 MHz, DMSO-d6) δ[ppm]: 1.138 (0.71), 2.518 (1.37), 2.523 (0.95), 3.921 (0.48), 3.941 (3.55), 3.954 (5.16), 3.959 (11.63), 3.963 (6.65), 3.968 (6.03), 3.975 (6.58), 4.001 (2.03), 4.030 (2.04), 4.054 (6.56), 4.062 (5.75), 4.067 (6.32), 4.072 (11.44), 4.077 (4.72), 4.089 (3.59), 4.109 (0.46), 5.963 (16.00), 7.529 (4.94), 7.552 (5.20), 7.555 (5.24), 7.577 (4.93), 7.774 (4.03), 7.794 (6.93), 7.813 (4.04).

### Intermediate 3

### 5-(1,3-dioxolan-2-yl)-2,4-difluorobenzonitrile

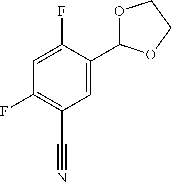 [SMILES: N#Cc1cc(C2OCCO2)c(F)cc1F]

A mixture of 2-(5-bromo-2,4-difluorophenyl)-1,3-dioxolane (12.1 g, approx. 75% purity, approx. 34.2 mmol) and (3.57 g, 39.9 mmol) copper cyanide in N-methylpyrrolidone (25 ml) was equally distributed to two microwave vials, and each vial was heated to 175° C. in a microwave oven for 75 min. Water (20 mL) and an aqueous solution of ammonium hydroxide (3 mL) were added and the mixture was extracted with ethyl acetate. The organic phase was washed with saturated sodium chloride solution, dried (sodium sulfate), filtered and the solvent was removed in vacuum. Silicagel chromatography gave 5.30 g of the title compound.

LC-MS (Method 1): Rₜ=0.95 min; MS (ESIpos): m/z=212 [M+H]⁺

¹H-NMR (400 MHz, DMSO-d6) δ[ppm]: 1.987 (0.69), 2.518 (1.54), 2.523 (1.03), 3.935 (0.42), 3.954 (3.49), 3.967 (4.78), 3.972 (11.09), 3.976 (6.39), 3.981 (5.82), 3.988 (6.81), 4.013 (2.30), 4.033 (2.23), 4.057 (6.79), 4.064 (5.56), 4.069 (6.09), 4.074 (10.54), 4.079 (4.91), 4.092 (3.42), 4.111 (0.48), 6.017 (16.00), 7.708 (4.28), 7.734 (6.58), 7.759 (4.22), 8.081 (3.64), 8.100 (7.21), 8.119 (3.62), 10.091 (0.43).

### Intermediate 4

### 5-(1,3-dioxolan-2-yl)-2,4-difluoro-3-iodobenzonitrile

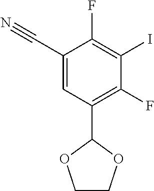 [SMILES: N#Cc1cc(C2OCCO2)c(F)c(I)c1F]

To a stirred solution of 2,2,6,6-tetramethylpiperidine (2.8 ml, 16 mmol) in THF (40 mL) in an ice bath was added n-butyl lithium in hexanes (6.1 ml, 2.5 M, 15 mmol) and the mixture was stirred at 0° C. for 0.5 h. This solution was slowly added to a stirred solution of 5-(1,3-dioxolan-2-yl)-2,4-difluorobenzonitrile (2.30 g, 10.9 mmol) in THF (120 mL) in a dry ice bath at approx. −78° C. and the mixture was stirred at −78° C. for further 0.5 h. Iodine (3.87 g, 15.2 mmol) was added and the mixture was stirred at −78° C. for 0.5 h. The dry ice bath was removed and the mixture was allowed to warm up to 0° C. Water and a n aqueous solution of disodium sulfurothioate were added, the mixture was stirred for 15 min and then the mixture was extracted with ethyl acetate. The organic phases were combined and washed with saturated sodium chloride solution, dried (sodium sulfate), filtered and the solvent was removed in vacuum. Silicagel chromatography gave 3.02 g (82% yield) of the title compound.

LC-MS (Method 1): Rₜ=1.16 min; MS (ESIpos): m/z=338 [M+H]⁺

¹H-NMR (400 MHz, DMSO-d6) δ[ppm]: 1.154 (1.94), 1.172 (3.94), 1.190 (1.87), 1.987 (6.73), 2.084 (0.63), 2.518 (2.27), 2.523 (1.75), 3.938 (0.40), 3.955 (3.54), 3.969 (4.62), 3.972 (8.90), 3.974 (7.88), 3.978 (6.00), 3.983 (5.80), 3.990 (7.49), 3.999 (0.61), 4.014 (2.74), 4.017 (1.83), 4.024 (2.48), 4.034 (1.50), 4.047 (7.24), 4.054 (5.69), 4.059 (5.79), 4.066 (9.09), 4.069 (4.67), 4.082 (3.46), 6.034 (16.00), 8.074 (3.73), 8.092 (7.51), 8.111 (3.81).

### Intermediate 5

### 2,4-difluoro-5-formyl-3-iodobenzonitrile

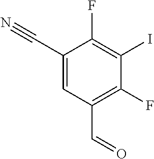 [SMILES: N#Cc1cc(C=O)c(F)c(I)c1F]

To a stirred solution of 5-(1,3-dioxolan-2-yl)-2,4-difluoro-3-iodobenzonitrile (3.01 g, 8.93 mmol) in acetone (81 ml) was added an aqueous solution of sulfuric acid (45 ml, 4.0 M, 180 mmol) and the mixture was stirred at 55° C. for 45 min. Water was added, the mixture was extracted with ethyl acetate. The organic phase was washed with saturated sodium chloride solution, dried (sodium sulfate), filtered and the solvent was removed in vacuu to give 2.59 g (99 yield) of the title compound as a crude product that was used without further purification.

¹H-NMR (400 MHz, DMSO-d6) δ[ppm]: 8.48 (t, 1H), 10.06 (s, 1H)

### Intermediate 6

### 4-(5-cyano-2,4-difluoro-3-iodophenyl)-2,6-dimethyl-1,4-dihydropyridine-3,5-dicarbonitrile

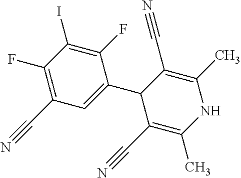 [SMILES: CC1=C(C#N)C(c2cc(C#N)c(F)c(I)c2F)C(C#N)=C(C)N1]

To a stirred solution of 2,4-difluoro-5-formyl-3-iodobenzonitrile (2.58 g, 8.81 mmol) in acetic acid (10 ml) was added (2E)-3-aminobut-2-enenitrile (1.63 g, 19.8 mmol) and the mixture was stirred at 90° C. for 1 h. The mixture was concentrated in vacuum. An aqueous solution of sodium bicarbonate was added and the mixture was extracted with ethyl acetate. The organic phase was washed with saturated sodium chloride solution, dried (sodium sulfate), filtered and the solvent was removed in vacuum. Silicagel chromatography gave a solid that was triturated with dichloromethane to give 2.65 g (71% yield) of the title compound.

LC-MS (Method 1): Rₜ=1.11 min; MS (ESIpos): m/z=422 [M+H]⁺

¹H-NMR (400 MHz, DMSO-d6) δ[ppm]: 2.042 (16.00), 2.518 (0.64), 2.523 (0.46), 4.881 (2.00), 5.756 (11.74), 8.117 (0.82), 8.136 (1.34), 8.156 (0.83), 9.742 (1.19).

### Intermediate 7

### 4-(5-cyano-2,4-difluoro-3-iodophenyl)-1,2,6-trimethyl-1,4-dihydropyridine-3,5-dicarbonitrile

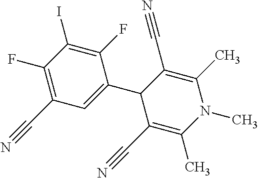 [SMILES: CC1=C(C#N)C(c2cc(C#N)c(F)c(I)c2F)C(C#N)=C(C)N1C]

To a stirred solution of 4-(5-cyano-2,4-difluoro-3-iodophenyl)-2,6-dimethyl-1,4-dihydropyridine-3,5-dicarbonitrile (870 mg, 2.06 mmol) in DMF (14 mL) was added potassium carbonate (2.1 g), and iodomethane (1.3 ml, 21 mmol) and the mixture was stirred at room temperature for 24 h. An aqueous solution of ammonium chloride was added, and the mixture was extracted with ethyl acetate. The organic phase was washed with a saturated solution of sodium bicarbonate and then with a saturated sodium chloride solution, dried (sodium sulfate), filtered and the solvent was removed in vacuum to give a a solid that was crystallized from ethanol to give 610 mg (67% yield) of the title compound.

LC-MS (Method 1): Rₜ=1.18 min; MS (ESIpos): m/z=436 [M+H]⁺

¹H-NMR (500 MHz, DMSO-d6) δ[ppm]: 1.987 (0.64), 2.239 (16.00), 2.515 (0.47), 2.518 (0.45), 3.329 (9.75), 4.828 (1.89), 5.758 (0.52), 8.042 (0.70), 8.057 (1.24), 8.072 (0.71).

### Intermediate 8

### 4-(3-amino-6-fluoro-7-iodo-1H-indazol-5-yl)-1,2,6-trimethyl-1,4-dihydropyridine-3,5-dicarbonitrile

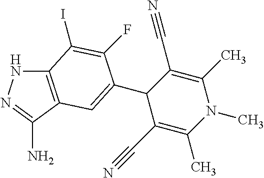 [SMILES: CC1=C(C#N)C(c2cc3c(N)n[nH]c3c(I)c2F)C(C#N)=C(C)N1C]

To a stirred solution of 4-(5-cyano-2,4-difluoro-3-iodophenyl)-1,2,6-trimethyl-1,4-dihydropyridine-3,5-dicarbonitrile (672 mg, 1.54 mmol) in 2-propanol (12 ml) in a microwave vial, was added hydrazine hydrate (530 μl, 65% purity, 15 mmol) and the mixture was stirred at 100° C. for 1 h. An aqueous solution of sodium bicarbonate was added, and the mixture was extracted with ethyl acetate. The organic phase was washed with saturated sodium chloride solution, dried (sodium sulfate), filtered and the solvent was removed in vacuum.

Aminophase-silicagel chromatography followed by crystallization from ethanol gave 467 mg (67% yield) of the title compound.

LC-MS (Method 1): Rₜ=0.89 min; MS (ESIpos): m/z=449 [M+H]⁺

¹H-NMR (400 MHz, DMSO-d6) δ[ppm]: 1.035 (0.84), 1.053 (1.45), 1.070 (0.85), 1.154 (0.45), 1.171 (0.90), 1.189 (0.43), 1.987 (1.61), 2.249 (16.00), 2.518 (0.69), 2.522 (0.46), 3.224 (10.60), 4.355 (0.43), 4.611 (1.59), 5.614 (1.97), 7.547 (1.57), 7.565 (1.57), 11.742 (1.46).

### Intermediate 9

### 5-(1,3-dioxolan-2-yl)-2,4-difluoro-3-methylbenzonitrile

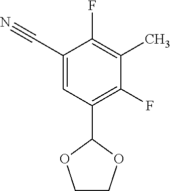 [SMILES: Cc1c(F)c(C#N)cc(C2OCCO2)c1F]

To a stirred solution of 2,2,6,6-tetramethylpiperidine (6.3 ml, 38 mmol) in THF (80 mL) in an ice bath was added n-butyl lithium in hexanes (14 ml, 2.5 M, 35 mmol) and the mixture was stirred at 0° C. for 0.5 h. This solution was slowly added to a stirred solution of 5-(1,3-dioxolan-2-yl)-2,4-difluorobenzonitrile (5.29 g, 25.1 mmol) in THF (160 mL) in a dry ice bath at approx. −78° C. and the mixture was stirred at −78° C. for further 0.5 h. Iodomethane (2.2 ml, 35 mmol) was added and the mixture was stirred at −78° C. for 0.5 h. The dry ice bath was removed and the mixture was allowed to warm up to 0° C. Water was added and the mixture was extracted with ethyl acetate. The organic phases were combined and washed with saturated sodium chloride solution, dried (sodium sulfate), filtered and the solvent was removed in vacuum. Silicagel chromatography gave 4.22 g (75% yield) of the title compound.

LC-MS (Method 1): Rₜ=1.10 min; MS (ESIpos): m/z=226 [M+H]⁺

¹H-NMR (400 MHz, DMSO-d6) δ[ppm]: 2.200 (8.98), 2.205 (16.00), 2.209 (9.05), 2.518 (0.84), 2.523 (0.62), 3.952 (2.86), 3.965 (4.04), 3.970 (8.15), 3.974 (4.86), 3.980 (4.59), 3.987 (5.66), 4.011 (2.01), 4.027 (1.95), 4.052 (5.71), 4.059 (4.73), 4.064 (4.99), 4.068 (7.71), 4.073 (3.72), 4.086 (2.75), 6.011 (12.55), 7.903 (2.02), 7.922 (3.95), 7.940 (2.02).

### Intermediate 10

### 2,4-difluoro-5-formyl-3-methylbenzonitrile

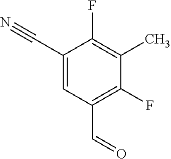

To a stirred solution of 5-(1,3-dioxolan-2-yl)-2,4-difluoro-3-methylbenzonitrile (4.22 g, 18.7 mmol) in acetone (170 ml) was added an aqueous solution of sulfuric acid (94 ml, 4.0 M, 370 mmol) and the mixture was stirred at 55° C. for 45 m in. Water was added, the mixture was extracted with ethyl acetate. The organic phase was washed with saturated sodium chloride solution, dried (sodium sulfate), filtered and the solvent was removed in vacuu to give 3.30 g (97% yield) of the title compound as a crude product that was used without any further purification.

LC-MS (Method 1): Rₜ=0.96 min; MS (ESIpos): m/z=182 [M+H]⁺

¹H-NMR (400 MHz, DMSO-d6) δ[ppm]: 1.136 (1.18), 1.906 (0.45), 2.114 (0.50), 2.125 (0.67), 2.199 (0.44), 2.205 (0.57), 2.248 (9.18), 2.252 (16.00), 2.257 (8.90), 2.518 (1.08), 2.522 (0.71), 8.317 (1.96), 8.335 (3.81), 8.354 (2.00), 10.107 (11.03).

### Intermediate 11

### 4-(5-cyano-2,4-difluoro-3-methylphenyl-2,6-dimethyl-1,4-dihydropyridine-3,5-dicarbonitrile

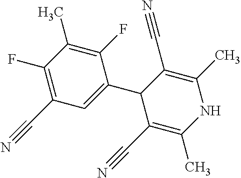 [SMILES: CC1=C(C#N)C(c2cc(C#N)c(F)c(C)c2F)C(C#N)=C(C)N1]

A mixture of 2,4-difluoro-5-formyl-3-methylbenzonitrile (3.30 g, 18.2 mmol) and (2E)-3-aminobut-2-enenitrile (3.37 g, 41.0 mmol) in acetic acid (21 ml) and N-methylpyrrolidone (9.9 ml) was equally distributed to two microwave vials, and each vial was heated to 90° C. in a microwave oven for 30 min. The reaction mixtures were combined and concentrated in vacuum. An aqueous solution of sodium bicarbonate was added and the mixture was extracted with ethyl acetate. The organic phase was washed with saturated sodium chloride solution, dried (sodium sulfate), filtered and the solvent was removed in vacuum. Silicagel chromatography gave a solid that was triturated with dichloromethane to give 4.01 g (71 yield) of the title compound.

LC-MS (Method 1): Rₜ=1.07 min; MS (ESIpos): m/z=311 [M+H]⁺

¹H-NMR (400 MHz, DMSO-d6) δ[ppm]: 2.035 (16.00), 2.230 (4.16), 2.518 (0.45), 4.823 (1.93), 7.921 (0.52), 7.940 (0.98), 7.959 (0.54), 9.701 (0.92).

### Intermediate 12

### 4-(5-cyano-2,4-difluoro-3-methylphenyl)-1,2,6-trimethyl-1,4-dihydropyridine-3,5-dicarbonitrile

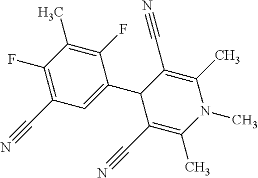 [SMILES: CC1=C(C#N)C(c2cc(C#N)c(F)c(C)c2F)C(C#N)=C(C)N1C]

To a stirred solution of 4-(5-cyano-2,4-difluoro-3-methylphenyl)-2,6-dimethyl-1,4-dihydropyridine-3,5-dicarbonitrile (1.30 g, 4.19 mmol) in THF (42 mL) was added sodium hydride in oil (274 mg, 55% purity, 6.28 mmol) at 0° C. and the mixture was stirred at room temperature for 30 min. Iodomethane (1.0 ml, 17 mmol) was added and the mixture was stirred at room temperature for 4 days. An aqueous solution of ammonium chloride was added, and the mixture was extracted with ethyl acetate. The organic phase was washed with a saturated sodium chloride solution, dried (sodium sulfate), filtered and the solvent was removed in vacuum. Silicagel chromatography gave 1.10 g (80% yield) of the title compound.

LC-MS (Method 1): Rₜ=1.14 min; MS (ESIpos): m/z=325 [M+H]⁺

¹H-NMR (400 MHz, DMSO-d6) δ[ppm]: 1.154 (0.58), 1.172 (1.17), 1.190 (0.57), 1.988 (2.09), 2.228 (4.74), 2.235 (16.00), 2.518 (0.56), 3.329 (9.37), 4.017 (0.46), 4.035 (0.45), 4.769 (1.74), 5.758 (0.62), 7.861 (0.50), 7.880 (0.93), 7.899 (0.52).

### Intermediate 13

### 4-(3-amino-6-fluoro-7-methyl-1H-indazol-5-yl)-1,2,6-trimethyl-1,4-dihydropyridine-3,5-dicarbonitrile

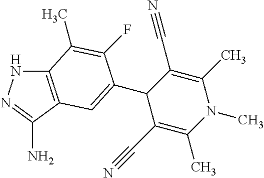 [SMILES: CC1=C(C#N)C(c2cc3c(N)n[nH]c3c(C)c2F)C(C#N)=C(C)N1C]

To a stirred solution of 4-(5-cyano-2,4-difluoro-3-methylphenyl)-1,2,6-trimethyl-1,4-dihydropyridine-3,5-dicarbonitrile (1.08 g, 3.31 mmol) in 2-propanol (20 ml) in a microwave vial, was added hydrazine hydrate (1.1 ml, 65% purity, 33 mmol) and the mixture was stirred at 130° C. for 3 h. An aqueous solution of sodium bicarbonate was added, and the mixture was extracted with ethyl acetate. The organic phase was washed with saturated sodium chloride solution, dried (sodium sulfate), filtered and the solvent was removed in vacuum. Aminophase-silicagel chromatography followed by crystallization from ethanol and trituration with dichloromethane gave 783 mg (70% yield) of the title compound.

LC-MS (Method 1): Rₜ=0.86 min; MS (ESIpos): m/z=337 [M+H]⁺

¹H-NMR (400 MHz, DMSO-d6) δ[ppm]: 2.242 (16.00), 2.289 (4.53), 2.292 (4.57), 2.518 (0.66), 2.523 (0.45), 3.333 (2.12), 4.533 (1.82), 5.466 (2.22), 5.759 (0.45), 7.375 (1.14), 7.392 (1.16), 11.618 (1.09).

### Intermediate 14

### 4-{[5-(3,5-dicyano-1,2,6-trimethyl-1,4-dihydropyridin-4-yl)-6-fluoro-7-methyl-1H-indazol-3-yl]carbamoyl}cubane-1-carboxylic Acid, Salt with Hydrochloric Acid

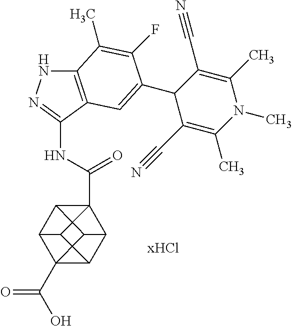 [SMILES: CC1=C(C#N)C(c2cc3c(NC(=O)C45C6C7C8(C(=O)O)C9C4C54C9C78C64)n[nH]c3c(C)c2F)C(C#N)=C(C)N1C]

To a stirred solution of methyl 4-{[5-(3,5-dicyano-1,2,6-trimethyl-1,4-dihydropyridin-4-yl)-6-fluoro-7-methyl-1H-indazol-3-yl]carbamoyl}cubane-1-carboxylate (290 mg, 553 μmol) in methanol (4.8 ml) was added an aqueous solution of sodium hydroxide (410 μl, 2.0 M, 830 μmol) and the mixture was stirred at r.t. for 2 h. Hydrochloric acid (c=4 N) was added until pH 3 was reached and the mixture was concentrated in vacuum until a solid precipitated. The solid was collected by filtration, washed with water and dried to give a solid that was triturated with a mixture of dichloromethane and hexane to give 311 mg (approx. 90% purity, approx. 93 yield) of the title compound as a crude product that was used without further purification.

LC-MS (Method 1): Rₜ=0.91 min; MS (ESIpos): m/z=511 [M+H]⁺

¹H-NMR (400 MHz, DMSO-d6) δ[ppm]: 2.228 (16.00), 2.327 (0.48), 2.394 (5.58), 2.522 (2.27), 2.669 (0.46), 3.333 (3.62), 4.145 (1.62), 4.287 (1.56), 4.601 (1.15), 5.759 (10.40), 7.520 (0.72), 7.537 (0.71), 10.375 (0.73).

### Intermediate 15

### 5-(1,3-dioxolan-2-yl)-3-ethyl-2,4-difluorobenzonitrile

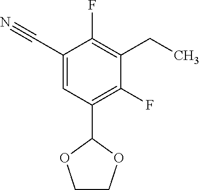 [SMILES: CCc1c(F)c(C#N)cc(C2OCCO2)c1F]

To a stirred solution of 5-(1,3-dioxolan-2-yl)-2,4-difluorobenzonitrile (2.00 g, 90% purity, 8.52 mmol) in THF (40 mL) was added n-butyl lithium in hexanes (4.1 ml, 2.5 M, 10 mmol) at −78° C. and the mixture was stirred at −78° C. for further 0.5 h. Then, iodoethane (3.99 g, 25.6 mmol) dissolved in THF was added at −78° C. and the mixture was slowly allowed to warm up to r.t. and was then stirred at r.t. for 0.5 h. A saturated solution of ammonium chloride was added and the mixture was extracted with ethyl acetate. The organic phases were combined and washed with water, saturated sodium chloride solution, dried (sodium sulfate), filtered and the solvent was removed in vacuum to give 2.10 g (88% yield) of the title compound as a brown oil that was used without further purification.

¹H-NMR (400 MHz, DMSO-d6) δ[ppm]: 1.14 (t, 3H), 2.67-2.70 (m, 2H), 3.92-4.07 (m, 4H), 6.02 (s, 1H), 7.91-7.96 (m, 1H).

### Intermediate 16

### 3-ethyl-2,4-difluoro-5-formylbenzonitrile

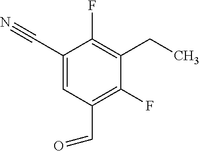

To a stirred solution of 5-(1,3-dioxolan-2-yl)-3-ethyl-2,4-difluorobenzonitrile (2.10 g, approx. 85% purity, approx. 7.46 mmol) in 1,4-dioxane (15 ml) was added an aqueous solution of hydrochloric acid (19 ml, 6.0 M, 110 mmol) at 0° C. and the mixture was stirred at r.t. for 6 h. A saturated solution of sodium bicarbonate was added until pH 7 was reached and the mixture was extracted with ethyl acetate. The organic phase was washed with water, saturated sodium chloride solution, dried (sodium sulfate), filtered and the solvent was removed in vacuu to give 1.50 g (88% yield) of the title compound as a brown oil that was used without further purification.

¹H-NMR (400 MHz, DMSO-d6) δ[ppm]: 1.19 (t, 3H), 2.75 (q, 2H), 8.34-8.38 (m, 1H), 10.12 (s, 1H).

### Intermediate 17

### 4-(5-cyano-3-ethyl-2,4-difluorophenyl)-2,6-dimethyl-1,4-dihydropyridine-3,5-dicarbonitrile

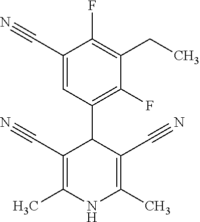 [SMILES: CCc1c(F)c(C#N)cc(C2C(C#N)=C(C)NC(C)=C2C#N)c1F]

To a stirred solution of 3-ethyl-2,4-difluoro-5-formylbenzonitrile (1.50 g, approx. 85% purity, approx. 6.53 mmol) in acetic acid (15 ml) was added and 3-aminobut-2-enenitrile (1.15 g, 14.0 mmol) and the mixture was stirred at 90° C. for 8 h. The mixture was concentrated in vacuum. An aqueous solution of sodium bicarbonate was added and the mixture was extracted with ethyl acetate. The organic phase was washed with saturated sodium chloride solution, dried (sodium sulfate), filtered and the solvent was removed in vacuum 1.60 g (68% yield) of the title compound as a light yellow solid.

LC-MS [Water (0.05% TFA)-Acetonitrile, 5% B]: Rₜ=1.12 min.

MS (ESIpos): m/z=325 (M+H)⁺.

¹H-NMR (400 MHz, DMSO-d6) δ[ppm]: 1.15 (t, 3H), 2.04 (s, 6H), 2.68 (q, 2H), 4.85 (s, 1H), 7.96 (t, 1H), 9.69 (s, 1H).

### Intermediate 18

### 4-(5-cyano-3-ethyl-2,4-difluorophenyl)-1,2,6-trimethyl-1,4-dihydropyridine-3,5-dicarbonitrile

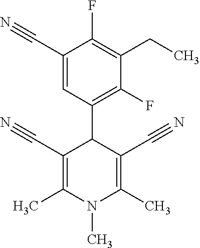 [SMILES: CCc1c(F)c(C#N)cc(C2C(C#N)=C(C)N(C)C(C)=C2C#N)c1F]

To a stirred solution of 4-(5-cyano-3-ethyl-2,4-difluorophenyl)-2,6-dimethyl-1,4-dihydropyridine-3,5-dicarbonitrile (1.60 g, approx. 80% purity, approx. 3.95 mmol) in THF (16 mL) was added potassium carbonate (1.64 g, 11.8 mmol), and iodomethane (1.2 ml, 20 mmol) and the mixture was stirred at room temperature for 38 h. Water was added, and the mixture was extracted with ethyl acetate. The organic phase was washed with a saturated sodium chloride solution, dried (sodium sulfate), filtered and the solvent was removed in vacuum Silicagel chromatography gave 1.40 g (84% yield) of the title compound as a yellow solid.

LC-MS [Water (0.1% FA)-Acetonitrile, 5% B]: Rₜ=1.13 min.

MS (ESIpos): m/z=339 (M+H)⁺.

¹H-NMR (400 MHz, DMSO-d6) δ[ppm]: 1.16 (t, 3H), 2.25 (s, 6H), 2.71 (q, 2H), 3.21 (s, 3H), 4.80 (s, 1H), 7.90 (t, 1H).

### Intermediate 19

### 4-(3-amino-7-ethyl-6-fluoro-1H-indazol-5-yl)-1,2,6-trimethyl-1,4-dihydropyridine-3,5-dicarbonitrile

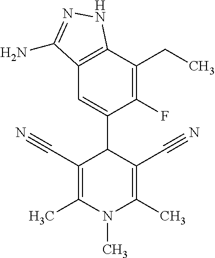 [SMILES: CCc1c(F)c(C2C(C#N)=C(C)N(C)C(C)=C2C#N)cc2c(N)n[nH]c12]

To a stirred solution of 4-(5-cyano-3-ethyl-2,4-difluorophenyl)-1,2,6-trimethyl-1,4-dihydropyridine-3,5-dicarbonitrile (1.40 g, 80% purity, 3.31 mmol) in 1-butanol (25 ml) was added hydrazine hydrate (432 mg, 98% purity, 13.2 mmol) and the mixture was stirred at 120° C. for 14 h. After cooled to room temperature, the solvent was removed in vacuo and the residue was washed with ethyl acetate/petroleum ether (v:v=1:5) to give 1.10 g (87% yield) of the title compound as a crude product that was used without further purification.

LC-MS [Water (0.05% TFA)-Acetonitrile, 5% B]: Rₜ=1.09 min.

MS (ESIpos): m/z=351 (M+H)⁺.

¹H-NMR (400 MHz, DMSO-d6) δ[ppm]: 1.17 (t, 3H), 2.25 (s, 6H), 2.78 (q, 2H), 3.22 (s, 3H), 4.56 (s, 1H), 5.46 (s, 2H), 7.38-7.39 (m, 1H), 11.63 (s, 1H).

### Intermediate 20

### 4-[5-cyano-2,4-difluoro-3-(trifluoromethyl)phenyl]-1,2,6-trimethyl-1,4-dihydropyridine-3,5-dicarbonitrile

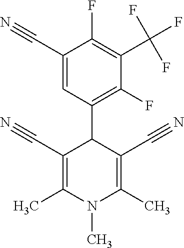 [SMILES: CC1=C(C#N)C(c2cc(C#N)c(F)c(C(F)(F)F)c2F)C(C#N)=C(C)N1C]

To a solution of 4-(5-cyano-2,4-difluoro-3-iodophenyl)-1,2,6-trimethyl-1,4-dihydropyridine-3,5-dicarbonitrile (400 mg, approx. 81% purity, approx. 743 μmol), in 15 mL of 1-methyl-2-pyrrolidinone/N,N-dimethylformamide (v:v=1:1) were added methyl 2,2-difluoro-2-(fluorosulfonyl)acetate, (1.14 g, 5.94 mmol), and copper(I) iodide (1.13 g, 5.94 mmol). The resulting mixture was stirred at 100° C. for 14 hours under nitrogen atmosphere. After cooling to room temperature, the solid was removed by filtration and the filtrate was diluted with water. The resulting solution was extracted with ethyl acetate. The combined organic phases were washed with water, saturated sodium chloride solution, dried (sodium sulfate), filtered and the solvent was removed in vacuum. Silicagel chromatography gave 250 mg (70% yield) of the title compound as a brown solid.

LC-MS [Water(0.1% FA)-Acetonitrile, 10% B]: Rₜ=1.57 min.

MS (ESIpos): m/z=379 (M+H)⁺.

¹H-NMR (400 MHz, DMSO-d6) δ[ppm]: 2.26 (s, 6H), 3.22 (s, 3H), 4.94 (s, 1H), 8.39-8.43

### Intermediate 21

### 4-[3-amino-6-fluoro-7-(trifluoromethyl)-1H-indazol-5-yl]-1,2,6-trimethyl-1,4-dihydropyridine-3,5-dicarbonitrile

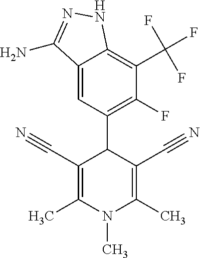 [SMILES: CC1=C(C#N)C(c2cc3c(N)n[nH]c3c(C(F)(F)F)c2F)C(C#N)=C(C)N1C]

To a stirred solution of 4-[5-cyano-2,4-difluoro-3-(trifluoromethyl)phenyl]-1,2,6-trimethyl-1,4-dihydropyridine-3,5-dicarbonitrile (250 mg, 80% purity, 529 μmol) in 1-butanol (20 ml) was added hydrazine hydrate (67.8 mg, 2.11 mmol) and the mixture was stirred at 120° C. for 14 h. After cooled to room temperature, the solvent was removed in vacuo and the residue was washed with ethyl acetate/petroleum ether (v:v=1:5) to give 300 mg (83% yield) of the title compound as a crude product that was used without further purification.

LC-MS [Water(0.1% FA)-Acetonitrile, 10% B]: Rₜ=1.23 min.

MS (ESIpos): m/z=391 (M+H)⁺.

¹H-NMR (400 MHz, DMSO-d6) δ[ppm]: 2.27-2.29 (s, 6H), 3.23 (s, 3H), 4.73 (s, 1H), 5.83 (s, 2H), 7.93 (d, 1H), 12.00 (s, 1H).

### Intermediate 22

### Ethyl 2-chloro-4-hydroxybenzoate

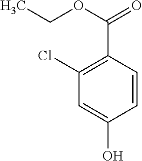

To a stirred solution of 2-chloro-4-hydroxybenzoic acid (3.50 g, 20.3 mmol) in ethanol (85 mL) was added thionyl dichloride (2.2 ml, 30 mmol). The mixture was heated to reflux for 4 h. The solvent was removed in vacuum. A solution of potassium carbonate (c=1 M) was added and the mixture was extracted with ethyl acetate. The organic phase was washed with saturated sodium chloride solution, dried (sodium sulfate) and the solvent was removed in vacuum to give 4.05 g (99% yield) of the title compound as a crude product that was used without further purification.

LC-MS (Method 1): Rₜ=0.98 min; MS (ESIpos): m/z=201 [M+H]⁺

¹H-NMR (400 MHz, DMSO-d6) δ[ppm]: 1.267 (7.36), 1.285 (16.00), 1.303 (7.37), 4.221 (2.36), 4.239 (7.49), 4.257 (7.36), 4.274 (2.23), 6.797 (2.89), 6.804 (3.16), 6.819 (2.74), 6.825 (3.42), 6.891 (5.81), 6.897 (4.97), 7.742 (5.03), 7.764 (4.88), 10.670 (10.31).

### Intermediate 23

### ethyl 2-chloro-4-[2-(pyrrolidin-1-yl)ethoxy]benzoate

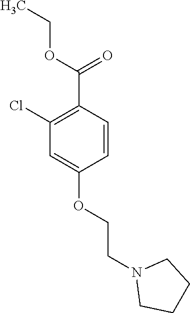 [SMILES: CCOC(=O)c1ccc(OCCN2CCCC2)cc1Cl]

To a stirred solution of ethyl 2-chloro-4-hydroxybenzoate (4.00 g, 19.9 mmol) in DMF (27 mL) was added potassium carbonate (13.8 g, 99.7 mmol), and 1-(2-chloroethyl)pyrrolidine-hydrogen chloride (4.50 g, 25.9 mmol) and the mixture was heated to 100° C. 2 h. Water was added, and the mixture was extracted with ethyl acetate. The organic phase was dried (sodium sulfate), filtered and the solvent was removed in vacuum. Silicagel chromatography gave 4.88 g (82% yield) of the title compound.

LC-MS (Method 1): Rₜ=0.74 min; MS (ESIpos): m/z=298 [M+H]⁺

¹H-NMR (400 MHz, DMSO-d6) δ[ppm]: 1.281 (7.52), 1.299 (16.00), 1.317 (7.65), 1.656 (3.21), 1.663 (4.87), 1.672 (9.64), 1.681 (4.84), 1.689 (3.34), 1.697 (0.58), 2.729 (0.58), 2.763 (3.68), 2.777 (7.87), 2.792 (3.82), 2.888 (0.65), 4.141 (3.89), 4.155 (7.79), 4.170 (3.66), 4.246 (2.39), 4.264 (7.39), 4.281 (7.27), 4.299 (2.28), 7.004 (2.48), 7.010 (2.65), 7.025 (2.58), 7.032 (2.88), 7.136 (5.47), 7.142 (4.96), 7.802 (5.60), 7.824 (5.16).

### Intermediate 24

### 2-chloro-4-[2-(pyrrolidin-1-yl)ethoxy]benzoic Acid

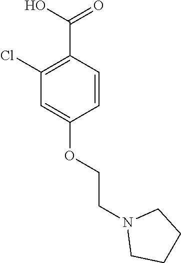 [SMILES: O=C(O)c1ccc(OCCN2CCCC2)cc1Cl]

To a stirred solution of ethyl 2-chloro-4-[2-(pyrrolidin-1-yl)ethoxy]benzoate (4.88 g, 16.4 mmol) in ethanol (100 ml) was added an aqueous solution of sodium hydroxide (12 ml, 2.0 M, 25 mmol) and the mixture was stirred at r.t. for 2 h. Hydrochloric acid (4 N) was added until pH 3 was reached and the mixture was concentrated in vacuum. Toluene was added and the mixture was concentrated again in vacuum. A mixture of methanol and dichloromethane was added and sodium sulfate was added and the mixture was stirred for 0.5 h. The mixture was filtered and the solvent was removed in vacuum to give 2.55 g (58% yield) of the title compound as a crude product that was used without further purification.

LC-MS (Method 1): Rₜ=0.49 min; MS (ESIpos): m/z=270 [M+H]⁺

¹H-NMR (400 MHz, DMSO-d6) δ[ppm]: 1.679 (0.65), 1.698 (5.55), 1.706 (8.38), 1.714 (16.00), 1.722 (8.22), 1.731 (5.57), 1.750 (0.60), 1.905 (0.74), 2.522 (0.92), 2.615 (6.05), 2.617 (6.13), 2.631 (13.36), 2.668 (0.59), 2.876 (5.49), 2.890 (11.11), 2.905 (5.59), 3.165 (4.82), 4.160 (6.12), 4.174 (11.82), 4.188 (5.77), 6.932 (4.22), 6.939 (4.58), 6.954 (4.29), 6.961 (4.76), 7.048 (9.32), 7.053 (8.28), 7.726 (9.15), 7.747 (8.51), 8.259 (0.42).

### Intermediate 25

### ethyl 4-[2-(pyrrolidin-1-yl)ethoxy]benzoate

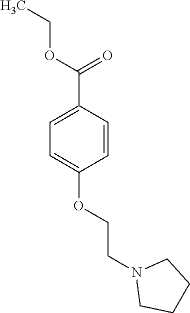 [SMILES: CCOC(=O)c1ccc(OCCN2CCCC2)cc1]

To a stirred solution of ethyl 4-hydroxybenzoate (5.00 g, 30.1 mmol) in DMF (40 mL) was added potassium carbonate (20.8 g, 150 mmol), and 1-(2-chloroethyl)pyrrolidine-hydrogen chloride (6.79 g, 98% purity, 39.1 mmol) and the mixture was heated to 100° C. for 2 h. Water was added, and the mixture was extracted with ethyl acetate. The organic phase was dried (sodium sulfate), filtered and the solvent was removed in vacuum. Aminophase-silicagel chromatography gave 7.30 g (92% yield) of the title compound.

LC-MS (Method 2): Rₜ=12.00 min; MS (ESIpos): m/z=264 [M+H]⁺

¹H-NMR (400 MHz, DMSO-d6) δ[ppm]: 1.281 (7.11), 1.299 (16.00), 1.317 (7.26), 1.657 (2.69), 1.662 (2.28), 1.665 (4.00), 1.670 (3.48), 1.674 (8.16), 1.683 (3.94), 1.691 (2.79), 2.520 (2.81), 2.523 (2.88), 2.776 (3.21), 2.791 (7.02), 2.805 (3.36), 4.121 (3.33), 4.136 (6.89), 4.150 (3.18), 4.239 (2.07), 4.257 (6.57), 4.275 (6.46), 4.293 (1.97), 7.030 (6.16), 7.035 (1.85), 7.047 (1.96), 7.052 (6.72), 7.882 (6.80), 7.887 (1.98), 7.899 (1.95), 7.904 (6.66).

### Intermediate 26

### 4-[2-(pyrrolidin-1-yl)ethoxy]benzoic acid salt, with hydrochloric Acid

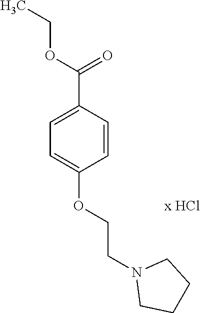 [SMILES: CCOC(=O)c1ccc(OCCN2CCCC2)cc1 ; "x HCl"]

To a stirred solution of ethyl 4-[2-(pyrrolidin-1-yl)ethoxy]benzoate (7.30 g, 27.7 mmol) in methanol methanol (70 ml) was added an aqueous solution of sodium hydroxide (17 ml, 5.0 M, 87 mmol) and the mixture was stirred at 50° C. for 4 h. The solvent was removed in vacuum. Hydrochloric acid was added until pH 6.5 was reached and the mixture was concentrated in vacuum. Toluene was added and the mixture was concentrated again in vacuum to give 10.2 g (approx. 64% purity) of the title compound as a crude product that was used without further purification.

LC-MS (Method 2): Rₜ=0.47 min; MS (ESIpos): m/z=236 [M+H]⁺

¹H-NMR (400 MHz, DMSO-d6) δ[ppm]: 1.657 (5.60), 1.664 (8.53), 1.673 (16.00), 1.682 (8.54), 1.690 (5.67), 1.698 (1.17), 2.327 (0.95), 2.523 (6.32), 2.669 (1.02), 2.748 (6.04), 2.763 (12.87), 2.778 (6.30), 3.155 (4.87), 3.343 (0.72), 4.026 (6.19), 4.041 (12.42), 4.055 (5.92), 6.764 (1.62), 6.771 (11.18), 6.776 (4.11), 6.788 (4.48), 6.793 (11.27), 6.800 (1.69), 7.761 (1.71), 7.768 (12.02), 7.773 (4.21), 7.785 (4.20), 7.790 (11.27), 7.797 (1.55).

### Intermediate 27

### ethyl 1-(2-methoxyethyl)-3-(trifluoromethyl)-1H-pyrazole-4-carboxylate

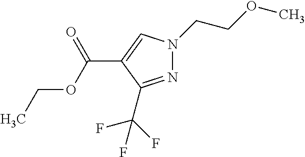 [SMILES: CCOC(=O)c1cn(CCOC)nc1C(F)(F)F]

To a stirred solution of ethyl 3-(trifluoromethyl)-1H-pyrazole-4-carboxylate (2.5 ml, 4.8 mmol) in THF (20 mL) was added sodium hydride in oil (266 mg, 65% purity, 7.21 mmol) at 0° C. and the mixture was stirred at room temperature for 30 min. 1-bromo-2-methoxyethane (900 μl, 9.6 mmol) was added and the mixture was stirred at 60° C. for 2 days. Water and an aqueous solution of sodium bicarbonate was added, and the mixture was extracted with ethyl acetate. The organic phase was washed with a saturated sodium chloride solution, dried (sodium sulfate), filtered and the solvent was removed in vacuum. Silicagel chromatography gave 960 mg (75% yield) of the title compound.

LC-MS (Method 1): Rₜ=1.11 min; MS (ESIpos): m/z=267 [M+H]⁺

¹H-NMR (400 MHz, DMSO-d6) δ[ppm]: 1.249 (7.26), 1.267 (16.00), 1.285 (7.34), 3.330 (3.16), 3.699 (2.95), 3.712 (4.23), 3.725 (3.18), 4.217 (2.24), 4.235 (7.27), 4.252 (7.14), 4.270 (2.13), 4.381 (2.57), 4.394 (3.86), 4.407 (2.36), 8.548 (3.50), 8.550 (3.44).

### Intermediate 28

### 1-(2-methoxyethyl)-3-(trifluoromethyl)-1H-pyrazole-4-carboxylic acid

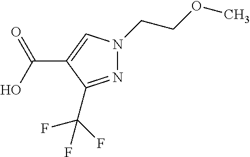 [SMILES: COCCn1cc(C(=O)O)c(C(F)(F)F)n1]

To a stirred solution of ethyl 1-(2-methoxyethyl)-3-(trifluoromethyl)-1H-pyrazole-4-carboxylate (1.07 g, 4.02 mmol) in ethanol (25 ml) was added an aqueous solution of sodium hydroxide (3.0 ml, 2.0 M, 6.0 mmol) and the mixture was stirred at r.t. for 2 h. Hydrochloric acid (4 N) was added until pH 3 was reached, water was added and the mixture was concentrated in vacuum to a volume of approx. 30 mL. The mixture was extracted with ethyl acetate. The organic phase was dried (sodium sulfate), filtered and the solvent was removed in vacuum. Silicagel chromatography gave 375 mg (39% yield) of the title compound.

LC-MS (Method 1): Rₜ=0.80 min; MS (ESIpos): m/z=239 [M+H]⁺

¹H-NMR (400 MHz, DMSO-d6) δ[ppm]: 1.232 (0.40), 1.250 (1.74), 1.267 (3.63), 1.285 (1.72), 2.518 (2.66), 2.522 (1.78), 2.673 (0.43), 3.055 (0.60), 3.191 (0.44), 3.333 (3.65), 3.408 (0.67), 3.696 (10.81), 3.709 (16.00), 3.722 (11.44), 4.217 (0.50), 4.234 (1.53), 4.252 (1.46), 4.271 (0.45), 4.363 (9.24), 4.377 (14.48), 4.389 (8.71), 4.406 (0.67), 5.758 (0.74), 8.455 (13.15), 8.457 (13.01), 8.548 (0.78), 8.551 (0.78), 12.943 (3.22).

### Intermediate 29

### ethyl 1-[2-(pyrrolidin-1-yl)ethyl]-3-(trifluoromethyl)-1H-pyrazole-4-carboxylate

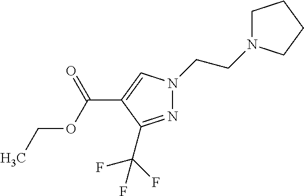 [SMILES: CCOC(=O)c1cn(CCN2CCCC2)nc1C(F)(F)F]

To a stirred solution of ethyl 3-(trifluoromethyl)-1H-pyrazole-4-carboxylate (550 mg, 2.64 mmol) in DMF (6 mL) was added sodium hydride in oil (346 mg, 55% purity, 7.93 mmol) at 0° C. and the mixture was stirred at room temperature for 30 min. 1-(2-chloroethyl)pyrrolidine-hydrogen chloride (674 mg, 3.96 mmol) was added and the mixture was stirred at room temperature for 3 days. Water and an aqueous solution of sodium bicarbonate was added, and the mixture was extracted with ethyl acetate. The organic phase was washed with a saturated sodium chloride solution, dried (sodium sulfate), filtered and the solvent was removed in vacuum. Silicagel chromatography gave 370 mg (45% yield) of the title compound and 40.0 mg (5 yield) of a second isomer, Intermediate 30.

LC-MS (Method 1): Rₜ=0.71 min; MS (ESIpos): m/z=306 [M+H]⁺

¹H-NMR (400 MHz, DMSO-d6) δ[ppm]: 1.27 (t, 3H), 1.65 (dt, 4H), 2.41-2.48 (m, 4H), 2.84 (t, 2H), 4.24 (q, 2H), 4.32 (t, 2H), 8.60 (d, 1H)

### Intermediate 30

### ethyl 1-[2-(pyrrolidin-1-yl)ethyl]-5-(trifluoromethyl)-1H-pyrazole-4-carboxylate

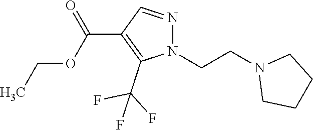 [SMILES: CCOC(=O)c1cnn(CCN2CCCC2)c1C(F)(F)F]

LC-MS (Method 1): Rₜ=0.67 min; MS (ESIpos): m/z=306 [M+H]⁺

¹H-NMR (400 MHz, DMSO-d6) δ[ppm]: 1.27 (t, 3H), 1.63 (dt, 4H), 2.39-2.46 (m, 4H), 2.81 (t, 2H), 4.25 (q, 2H), 4.45 (t, 2H), 8.08 (s, 1H)

### Intermediate 31

### 1-[2-(pyrrolidin-1-yl)ethyl]-3-(trifluoromethyl)-1H-pyrazole-4-carboxylic acid

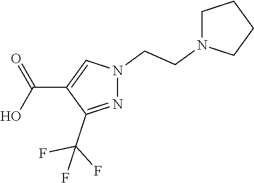 [SMILES: O=C(O)c1cn(CCN2CCCC2)nc1C(F)(F)F]

To a stirred solution of ethyl 1-[2-(pyrrolidin-1-yl)ethyl]-3-(trifluoromethyl)-1H-pyrazole-4-carboxylate (340 mg, 1.11 mmol) in ethanol (6.9 ml) was added an aqueous solution of sodium hydroxide (840 μl, 2.0 M, 1.7 mmol) and the mixture was stirred at r.t. for 2 h. Hydrochloric acid (4 N) was added until pH 3 was reached and the mixture was concentrated in vacuum. Toluene was added and the mixture was concentrated again in vacuum to give 1.50 g of the title compound as a crude product that was used without further purification.

LC-MS (Method 1): Rₜ=0.48 min; MS (ESIpos): m/z=278 [M+H]⁺

¹H-NMR (400 MHz, DMSO-d6) δ[ppm]: 1.263 (0.47), 1.617 (0.53), 1.636 (5.22), 1.644 (7.83), 1.653 (16.00), 1.662 (7.72), 1.670 (5.37), 1.677 (1.06), 1.689 (0.58), 1.901 (1.51), 2.442 (5.98), 2.446 (5.75), 2.459 (14.17), 2.472 (5.73), 2.476 (6.05), 2.478 (7.94), 2.518 (1.77), 2.523 (1.22), 2.725 (0.96), 2.727 (0.90), 2.805 (6.20), 2.821 (13.89), 2.837 (6.39), 2.888 (1.15), 3.159 (0.51), 4.230 (0.42), 4.244 (5.26), 4.260 (10.73), 4.276 (4.97), 5.761 (1.60), 8.215 (7.01), 8.376 (1.79).

### Intermediate 32

### 1-[2-(pyrrolidin-1-yl)ethyl]-5-(trifluoromethyl)-1H-pyrazole-4-carboxylic Acid

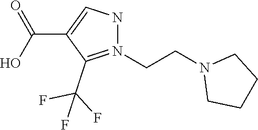

To a stirred solution of ethyl 1-[2-(pyrrolidin-1-yl)ethyl]-5-(trifluoromethyl)-1H-pyrazole-4-carboxylate (35.0 mg, 115 μmol) in ethanol (710 μl) was added an aqueous solution of sodium hydroxide (86 μl, 2.0 M, 170 μmol) and the mixture was stirred at r.t. for 2 h. Hydrochloric acid (4 N) was added until pH 3 was reached and the mixture was concentrated in vacuum. Toluene was added and the mixture was concentrated again in vacuum to give 65 mg of the title compound as a crude product that was used without further purification.

¹H-NMR (400 MHz, DMSO-d6) δ[ppm]: 1.230 (0.86), 1.695 (11.92), 1.907 (0.71), 2.337 (1.49), 2.456 (3.06), 2.461 (3.53), 2.518 (16.00), 2.523 (11.76), 2.600 (4.55), 2.975 (2.43), 3.155 (2.20), 3.165 (2.12), 4.482 (2.98), 4.499 (5.41), 4.514 (2.82), 4.653 (0.55), 5.761 (4.16), 8.013 (12.63), 8.065 (1.49).

### Intermediate 33

### ethyl 1-(2-hydroxy-2-methylpropyl)-3-(trifluoromethyl)-1H-pyrazole-4-carboxylate

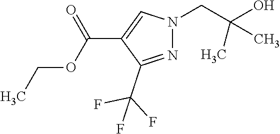 [SMILES: CCOC(=O)c1cn(CC(C)(C)O)nc1C(F)(F)F]

To a stirred solution of ethyl 3-(trifluoromethyl)-1H-pyrazole-4-carboxylate (2.5 ml, 2.4 mmol) in N,N-dimethylacetamide (3.0 ml, 32 mmol) was added potassium carbonate (766 mg, 65 purity, 3.60 mmol) and 1-chloro-2-methylpropan-2-ol (490 μl, 4.8 mmol) and the mixture was stirred at 80° C. for 10 h. Water was added, and the mixture was extracted with ethyl acetate.

The organic phase was washed with a saturated sodium chloride solution, dried (sodium sulfate), filtered and the solvent was removed in vacuum.

Silicagel chromatography gave 470 mg (69% yield) of the title compound and 33.0 mg (5 yield) of a second isomer, Intermediate 34.

LC-MS (Method 1): Rₜ=1.04 min; MS (ESIpos): m/z=281 [M+H]⁺

¹H-NMR (400 MHz, DMSO-d6) δ[ppm]: 1.07 (s, 6H), 1.27 (t, 3H), 4.14 (s, 2H), 4.19-4.30 (m, 2H), 4.85 (s, 1H), 8.37 (d, 1H).

### Intermediate 34

### ethyl 1-(2-hydroxy-2-methylpropyl)-5-(trifluoromethyl)-1H-pyrazole-4-carboxylate

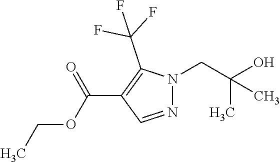 [SMILES: CCOC(=O)c1cnn(CC(C)(C)O)c1C(F)(F)F]

LC-MS (Method 1): Rₜ=1.04 min; MS (ESIpos): m/z=281 [M+H]⁺

¹H-NMR (400 MHz, DMSO-d6) δ[ppm]: 1.09 (s, 6H), 1.27 (t, 3H), 4.21-4.29 (m, 4H), 4.77 (s, 1H), 8.08 (s, 1H)

### Intermediate 35

### 1-(2-hydroxy-2-methylpropyl)-3-(trifluoromethyl)-1H-pyrazole-4-carboxylic Acid

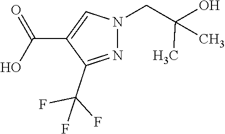 [SMILES: CC(C)(O)Cn1cc(C(=O)O)c(C(F)(F)F)n1]

To a stirred solution of ethyl 1-(2-hydroxy-2-methylpropyl)-3-(trifluoromethyl)-1H-pyrazole-4-carboxylate (410 mg, 1.46 mmol) in ethanol (9.1 ml) was added an aqueous solution of sodium hydroxide (1.1 ml, 2.0 M, 2.2 mmol) and the mixture was stirred at r.t. for 2 h. Hydrochloric acid (4 N) was added until pH 3 was reached water was added and the mixture was extracted with ethyl acetate. The organic phase was dried (sodium sulfate), filtered and the solvent was removed in vacuum to give 297 mg (80% yield) of the title compound as a crude product that was used without further purification.

LC-MS (Method 1): Rₜ=0.76 min; MS (ESIpos): m/z=253 [M+H]⁺

¹H-NMR (400 MHz, DMSO-d6) δ[ppm]: 1.073 (16.00), 1.907 (0.63), 3.336 (0.45), 4.124 (4.38), 4.841 (0.70), 8.292 (1.87), 8.295 (1.84).

### Intermediate 36

### 1-(2-hydroxy-2-methylpropyl)-5-(trifluoromethyl)-1H-pyrazole-4-carboxylic Acid

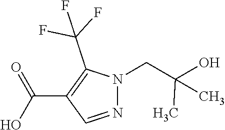 [SMILES: CC(C)(O)Cn1ncc(C(=O)O)c1C(F)(F)F]

To a stirred solution of ethyl 1-(2-hydroxy-2-methylpropyl)-5-(trifluoromethyl)-1H-pyrazole-4-carboxylate (30.0 mg, 107 μmol) in ethanol (670 μl) was added an aqueous solution of sodium hydroxide (80 μl, 2.0 M, 160 μmol) and the mixture was stirred at r.t. for 2 h. Hydrochloric acid (4 N) was added until pH 3 was reached and the mixture was concentrated in vacuum. Toluene was added and the mixture was concentrated in vacuum, then dichloromethane was added and the mixture was concentrated again in vacuum to give 63 mg of the title compound as a crude product that was used without further purification.

LC-MS (Method 1): Rₜ=0.72 min; MS (ESIneg): m/z=251 [M−H]⁻

¹H-NMR (400 MHz, DMSO-d6) δ[ppm]: 1.086 (16.00), 2.518 (1.22), 2.522 (0.86), 3.154 (0.42), 3.167 (0.42), 4.257 (3.37), 4.773 (3.61), 8.010 (2.32).

### EXPERIMENTAL SECTION—EXAMPLES

### Example 1

### N-[5-(3,5-dicyano-1,2,6-trimethyl-1,4-dihydropyridin-4-yl)-6-fluoro-7-iodo-1H-indazol-3-yl]-1-ethyl-3-methyl-1H-pyrazole-5-carboxamide

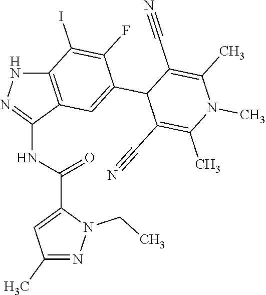 [SMILES: CCn1nc(C)cc1C(=O)Nc1n[nH]c2c(I)c(F)c(C3C(C#N)=C(C)N(C)C(C)=C3C#N)cc12]

To a stirred solution of 1-ethyl-3-methyl-1H-pyrazole-5-carboxylic acid (92.5 mg, 600 μmol) in dichloromethane (2.6 mL) was added DMF (2.3 μl, 30 μmol) and ethanedioyl dichloride (47 μl, 540 μmol) and the mixture was stirred at reflux for 2 h and then allowed to cool down to r.t. The crude reaction mixture was added to a stirred solution of 4-(3-amino-6-fluoro-7-iodo-1H-indazol-5-yl)-1,2,6-trimethyl-1,4-dihydropyridine-3,5-dicarbonitrile (175 mg, 390 μmol), pyridine (95 μl, 1.2 mmol) and DMAP (4.77 mg, 39.0 μmol) in dichloromethane (19 ml) and the mixture was stirred at r.t. for 1 h. Ethanol (0.2 mL) was added and the mixture was stirred at r.t. for 0.5 h. Aminophase-silicagel chromatography of the crude mixture followed by silicagel chromatography gave a solid that was triturated with dichloromethane to give 103 mg (45 yield) of the title compound.

LC-MS (Method 1): Rₜ=1.08 min; MS (ESIpos): m/z=585 [M+H]⁺

¹H-NMR (400 MHz, DMSO-d6) δ[ppm]: 1.035 (0.65), 1.053 (1.37), 1.071 (0.63), 1.317 (2.68), 1.335 (6.02), 1.353 (2.68), 2.216 (9.12), 2.236 (16.00), 2.518 (0.96), 2.523 (0.68), 3.331 (11.19), 4.355 (0.43), 4.450 (0.56), 4.468 (1.69), 4.486 (1.66), 4.503 (0.54), 4.726 (1.86), 5.758 (1.63), 6.974 (1.57), 7.702 (1.38), 7.719 (1.37), 10.870 (1.05), 13.214 (0.76).

### Example 2

### 2-chloro-N-[5-(3,5-dicyano-1,2,6-trimethyl-1,4-dihydropyridin-4-yl)-6-fluoro-7-iodo-1H-indazol-3-yl]benzamide

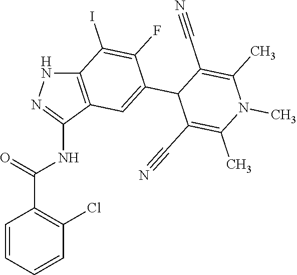 [SMILES: CC1=C(C#N)C(c2cc3c(NC(=O)c4ccccc4Cl)n[nH]c3c(I)c2F)C(C#N)=C(C)N1C]

To a stirred solution of 4-(3-amino-6-fluoro-7-iodo-1H-indazol-5-yl)-1,2,6-trimethyl-1,4-dihydropyridine-3,5-dicarbonitrile (20.0 mg, 44.6 μmol), pyridine (11 μl, 130 μmol) and DMAP (550 μg, 4.5 μmol) in dichloromethane (2.2 ml) at r.t. was added 2-chlorobenzoyl chloride (8.5 μl, 67 μmol) and the mixture was stirred at r.t. for 1 h. Ethanol (0.2 mL) was added and the mixture was stirred at r.t. for 0.5 h. Aminophase-silicagel chromatography of the crude mixture followed by silicagel chromatography gave 13.0 mg (45% yield) of the title compound.

LC-MS (Method 1): Rₜ=1.08 min; MS (ESIpos): m/z=587 [M+H]⁺

¹H-NMR (400 MHz, DMSO-d6) δ[ppm]: 1.035 (2.14), 1.053 (5.01), 1.071 (2.18), 1.233 (0.46), 2.085 (3.26), 2.246 (16.00), 2.270 (0.49), 2.331 (0.43), 2.518 (2.56), 2.523 (1.71), 2.674 (0.42), 3.214 (8.08), 3.423 (1.00), 3.435 (1.03), 3.440 (0.92), 3.453 (0.94), 4.344 (0.68), 4.356 (1.29), 4.369 (0.63), 4.712 (1.99), 5.759 (2.83), 7.477 (0.80), 7.495 (0.67), 7.512 (0.49), 7.532 (0.73), 7.549 (0.42), 7.588 (1.04), 7.607 (0.63), 7.661 (0.83), 7.676 (0.71), 7.785 (0.88), 7.803 (0.85), 11.097 (0.73), 13.165 (0.56).

### Example 3

### N-[5-(3,5-dicyano-1,2,6-trimethyl-1,4-dihydropyridin-4-yl)-6-fluoro-7-iodo-1H-indazol-3-yl]-1-methyl-3-(trifluoromethyl)-1H-pyrazole-4-carboxamide

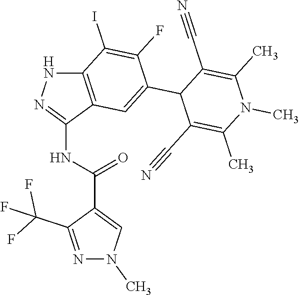 [SMILES: CC1=C(C#N)C(c2cc3c(NC(=O)c4cn(C)nc4C(F)(F)F)n[nH]c3c(I)c2F)C(C#N)=C(C)N1C]

To a stirred solution of 4-(3-amino-6-fluoro-7-iodo-1H-indazol-5-yl)-1,2,6-trimethyl-1,4-dihydropyridine-3,5-dicarbonitrile (100 mg, 223 μmol), pyridine (54 μl, 670 μmol) and DMAP (2.73 mg, 22.3 μmol) in dichloromethane (11 ml) at r.t. was added 1-methyl-3-(trifluoromethyl)-1H-pyrazole-4-carbonyl chloride (71.1 mg, 335 μmol) and the mixture was stirred at r.t. for 1 h. A half-saturated solution of sodium bicarbonate was added and the mixture was extracted with ethyl acetate. The organic phase was washed with saturated sodium chloride solution, dried (sodium sulfate) and the solvent was removed in vacuum. Aminophase-silicagel chromatography gave a solid that was triturated with dichloromethane to give 66.0 mg (47 yield) of the title compound.

LC-MS (Method 1): Rₜ=1.09 min; MS (ESIpos): m/z=625 [M+H]⁺

¹H-NMR (400 MHz, DMSO-d6) δ[ppm]: 2.236 (16.00), 2.518 (0.87), 2.523 (0.56), 3.331 (11.59), 3.991 (7.58), 4.677 (2.04), 5.758 (2.53), 7.771 (1.29), 7.789 (1.29), 8.638 (1.47), 10.939 (1.18), 13.137 (0.88).

### Example 4

### 2-chloro-N-[5-(3,5-dicyano-1,2,6-trimethyl-1,4-dihydropyridin-4-yl)-6-fluoro-7-iodo-1H-indazol-3-yl]-4-[2-(pyrrolidin-1-yl)ethoxy]benzamide

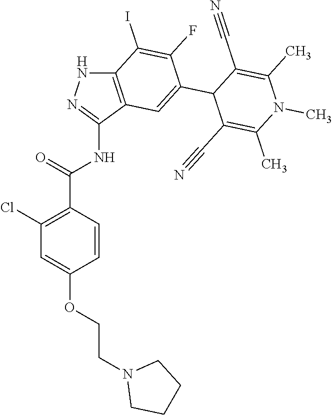 [SMILES: CC1=C(C#N)C(c2cc3c(NC(=O)c4ccc(OCCN5CCCC5)cc4Cl)n[nH]c3c(I)c2F)C(C#N)=C(C)N1C]

To a stirred solution of 4-(3-amino-6-fluoro-7-iodo-1H-indazol-5-yl)-1,2,6-trimethyl-1,4-dihydropyridine-3,5-dicarbonitrile (100 mg, 223 μmol) in DMA (3.0 mL) was added N,N-diisopropylethylamine (160 μl, 890 μmol), 2-chloro-4-[2-(pyrrolidin-1-yl)ethoxy]benzoic acid (72.2 mg, 268 μmol) and HATU (119 mg, 312 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum.

Aminophase-silicagel chromatography followed by silicagel chromatography gave 22.0 mg (14 yield) of the title compound.

LC-MS (Method 1): Rₜ=0.84 min; MS (ESIpos): m/z=700 [M+H]⁺

¹H-NMR (400 MHz, DMSO-d6) δ[ppm]: 1.053 (0.56), 1.703 (2.86), 2.244 (16.00), 2.518 (2.36), 2.523 (1.63), 2.562 (1.21), 2.766 (0.53), 2.840 (0.63), 2.979 (0.42), 3.215 (9.03), 4.173 (1.07), 4.707 (2.03), 5.759 (4.56), 7.027 (0.42), 7.044 (0.42), 7.169 (0.72), 7.597 (0.40), 7.773 (0.52), 7.790 (0.49), 10.953 (0.58), 13.138 (0.91).

### Example 5

### N-[5-(3,5-dicyano-1,2,6-trimethyl-1,4-dihydropyridin-4-yl)-6-fluoro-7-methyl-1H-indazol-3-yl]-1-ethyl-3-methyl-1H-pyrazole-5-carboxamide

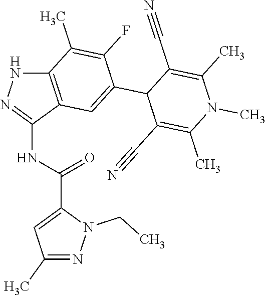 [SMILES: CCn1nc(C)cc1C(=O)Nc1n[nH]c2c(C)c(F)c(C3C(C#N)=C(C)N(C)C(C)=C3C#N)cc12]

To a stirred solution of 1-ethyl-3-methyl-1H-pyrazole-5-carboxylic acid (99.4 mg, 644 μmol) in dichloromethane (2.8 mL) was added DMF (2.5 μl, 32 μmol) and ethanedioyl dichloride (51 μl, 580 μmol) and the mixture was stirred at reflux for 2 h and then allowed to cool down to r.t. The crude reaction mixture was added to a stirred solution of 4-(3-amino-6-fluoro-7-methyl-1H-indazol-5-yl)-1,2,6-trimethyl-1,4-dihydropyridine-3,5-dicarbonitrile (130 mg, 386 μmol), pyridine (94 μl, 1.2 mmol) and DMAP (4.72 mg, 38.6 μmol) in dichloromethane (19 ml) and the mixture was stirred at r.t. for 1 h. Ethanol (0.2 mL) was added and the mixture was stirred at r.t. for 0.5 h. Aminophase-silicagel chromatography of the crude mixture followed by preparative reverse phase HPLC (Chromatex C18, 10 μm, 125×30 mm, gradient of water and acetonitrile containing 0.2% aqueous ammonia as additive) gave 86.0 mg (47% yield) of the title compound after lyophilisation.

LC-MS (Method 1): Rₜ=1.00 min; MS (ESIpos): m/z=473 [M+H]⁺

¹H-NMR (400 MHz, DMSO-d6) δ[ppm]: 1.317 (2.69), 1.334 (6.19), 1.352 (2.70), 2.214 (9.55), 2.226 (16.00), 2.423 (4.33), 2.426 (4.47), 2.518 (1.20), 2.523 (0.85), 3.202 (9.84), 4.451 (0.57), 4.469 (1.76), 4.487 (1.73), 4.505 (0.56), 4.641 (1.87), 6.968 (1.39), 7.513 (1.02), 7.530 (1.04), 10.764 (1.52), 13.094 (1.31).

### Example 6

### 2-chloro-N-[5-(3,5-dicyano-1,2,6-trimethyl-1,4-dihydropyridin-4-yl)-6-fluoro-7-methyl-1H-indazol-3-yl]benzamide

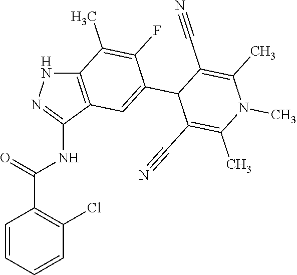 [SMILES: CC1=C(C#N)C(c2cc3c(NC(=O)c4ccccc4Cl)n[nH]c3c(C)c2F)C(C#N)=C(C)N1C]

To a stirred solution of 4-(3-amino-6-fluoro-7-methyl-1H-indazol-5-yl)-1,2,6-trimethyl-1,4-dihydropyridine-3,5-dicarbonitrile (50.0 mg, 149 μmol), pyridine (36 μl, 450 μmol) and DMAP (1.82 mg, 14.9 μmol) in dichloromethane (7.4 ml) at r.t. was added 2-chlorobenzoyl chloride (28 μl, 220 μmol) and the mixture was stirred at r.t. for 1 h. Ethanol (0.2 mL) was added, the mixture was stirred at r.t. for 0.5 h and the solvents were removed in vacuum. Preparative reverse phase HPLC (Chromatex C18, 10 μm, 125×30 mm, gradient of water and acetonitrile containing 0.2% aqueous ammonia as additive) gave 18.0 mg (25% yield) of the title compound after lyophilisation.

LC-MS (Method 1): Rₜ=1.04 min; MS (ESIpos): m/z=475 [M+H]⁺

¹H-NMR (400 MHz, DMSO-d6) δ[ppm]: 2.085 (5.05), 2.237 (16.00), 2.428 (4.34), 2.518 (1.28), 2.523 (0.96), 2.540 (0.87), 3.209 (8.27), 4.627 (2.09), 5.759 (9.67), 7.473 (0.92), 7.492 (0.77), 7.502 (0.54), 7.507 (0.57), 7.523 (0.82), 7.526 (0.87), 7.540 (0.47), 7.582 (1.25), 7.601 (0.81), 7.615 (1.00), 7.632 (1.03), 7.644 (1.01), 7.648 (0.97), 7.663 (0.79), 10.972 (1.56), 13.038 (1.37).

### Example 7

### N-[5-(3,5-dicyano-1,2,6-trimethyl-1,4-dihydropyridin-4-yl)-6-fluoro-7-methyl-1H-indazol-3-yl]-2,6-difluorobenzamide

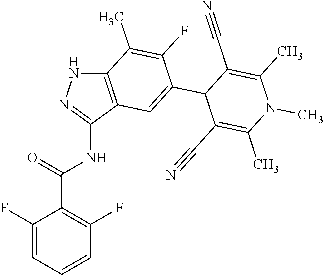 [SMILES: CC1=C(C#N)C(c2cc3c(NC(=O)c4c(F)cccc4F)n[nH]c3c(C)c2F)C(C#N)=C(C)N1C]

To a stirred solution of 4-(3-amino-6-fluoro-7-methyl-1H-indazol-5-yl)-1,2,6-trimethyl-1,4-dihydropyridine-3,5-dicarbonitrile (100 mg, 297 μmol), pyridine (72 μl, 890 μmol) and DMAP (3.63 mg, 29.7 μmol) in dichloromethane (15 ml) at r.t. was added 2,6-difluorobenzoyl chloride (56 μl, 450 μmol) and the mixture was stirred at r.t. for 14 h. Ethanol (0.2 mL) was added, the mixture was stirred at r.t. for 0.5 h and the solvents were removed in vacuum. Aminophase-silicagel chromatography followed by preparative reverse phase HPLC (Chromatex C18, 10 μm, 125×30 mm, gradient of water and acetonitrile containing 0.2% aqueous ammonia as additive) gave 13.0 mg (8% yield) of the title compound after lyophilisation.

LC-MS (Method 1): Rₜ=1.02 min; MS (ESIpos): m/z=477 [M+H]⁺

¹H-NMR (400 MHz, DMSO-d6) δ[ppm]: 2.239 (16.00), 2.275 (1.01), 2.430 (4.38), 2.518 (1.36), 2.523 (0.94), 2.540 (1.03), 3.212 (9.55), 3.255 (0.54), 4.622 (1.96), 5.759 (2.73), 7.253 (1.15), 7.273 (2.00), 7.293 (1.36), 7.536 (1.02), 7.554 (1.04), 7.583 (0.49), 7.587 (0.43), 7.603 (0.78), 7.625 (0.43), 11.270 (1.80), 13.104 (1.43).

### Example 8

### N-[5-(3,5-dicyano-1,2,6-trimethyl-1,4-dihydropyridin-4-yl)-6-fluoro-7-methyl-1H-indazol-3-yl]-2-ethylbenzamide

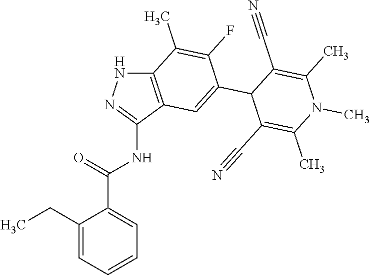 [SMILES: CCc1ccccc1C(=O)Nc1n[nH]c2c(C)c(F)c(C3C(C#N)=C(C)N(C)C(C)=C3C#N)cc12]

To a stirred solution of 4-(3-amino-6-fluoro-7-methyl-1H-indazol-5-yl)-1,2,6-trimethyl-1,4-dihydropyridine-3,5-dicarbonitrile (3.00 g, 8.92 mmol) in DMA (88 mL) was added N,N-diisopropylethylamine (6.2 ml, 36 mmol), 2-ethylbenzoic acid (2.01 g, 13.4 mmol) and HATU (5.43 g, 14.3 mmol). The mixture was stirred at 100° C. for 0.5 h. An aqueous solution of sodium bicarbonate was added, the mixture was stirred for 15 minutes and the mixture was extracted with ethyl acetate. The organic phase was washed with saturated sodium chloride solution, dried (sodium sulfate), filtered and the solvent was removed in vacuum.

Silicagel chromatography followed by aminophase-silicagel chromatography gave a solid that was triturated with a mixture of dichloromethane and hexane to give 3.06 g (73% yield) of the title compound.

LC-MS (Method 2): Rₜ=1.11 min; MS (ESIpos): m/z=469.5 [M+H]⁺

¹H-NMR (400 MHz, DMSO-d6) δ[ppm]: 0.831 (0.92), 0.837 (0.45), 0.854 (0.55), 0.859 (0.62), 1.195 (1.88), 1.214 (4.10), 1.233 (1.97), 1.395 (0.59), 2.234 (16.00), 2.425 (3.33), 2.518 (0.86), 2.523 (0.56), 2.814 (0.96), 2.833 (0.92), 3.205 (9.22), 4.639 (1.87), 7.311 (0.48), 7.338 (0.55), 7.358 (0.69), 7.430 (0.48), 7.520 (0.46), 7.537 (0.41), 7.570 (0.61), 7.587 (0.61), 10.768 (0.60), 12.992 (1.06).

### Example 9

### N-[5-(3,5-dicyano-1,2,6-trimethyl-1,4-dihydropyridin-4-yl)-6-fluoro-7-methyl-1H-indazol-3-yl]-1-methyl-3-(trifluoromethyl)-1H-pyrazole-4-carboxamide

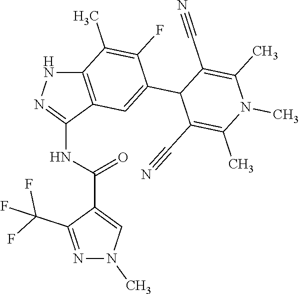 [SMILES: CC1=C(C#N)C(c2cc3c(NC(=O)c4cn(C)nc4C(F)(F)F)n[nH]c3c(C)c2F)C(C#N)=C(C)N1C]

To a stirred solution of 4-(3-amino-6-fluoro-7-methyl-1H-indazol-5-yl)-1,2,6-trimethyl-1,4-dihydropyridine-3,5-dicarbonitrile (100 mg, 297 μmol), pyridine (72 μl, 890 μmol) and DMAP (3.63 mg, 29.7 μmol) in dichloromethane (15 ml) at r.t. was added 1-methyl-3-(trifluoromethyl)-1H-pyrazole-4-carbonyl chloride (94.8 mg, 446 μmol) and the mixture was stirred at r.t. for 1 h. Ethanol (0.2 mL) was added, the mixture was stirred at r.t. for 0.5 h and the solvents were removed in vacuum. Aminophase-silicagel chromatography gave a solid that was crystallized from dichloromethane to give 55.0 mg (32% yield) of the title compound.

LC-MS (Method 1): Rₜ=1.01 min; MS (ESIpos): m/z=513 [M+H]⁺

¹H-NMR (400 MHz, DMSO-d6) δ[ppm]: 2.227 (16.00), 2.413 (4.27), 2.416 (4.35), 2.518 (0.89), 2.522 (0.61), 3.202 (10.18), 3.988 (7.54), 4.593 (1.99), 7.585 (0.98), 7.602 (0.99), 8.626 (1.26), 10.820 (1.70), 13.018 (1.56).

### Example 10

### N-[5-(3,5-dicyano-1,2,6-trimethyl-1,4-dihydropyridin-4-yl)-6-fluoro-7-methyl-1H-indazol-3-yl]-5-fluoro-2-(trifluoromethyl)benzamide

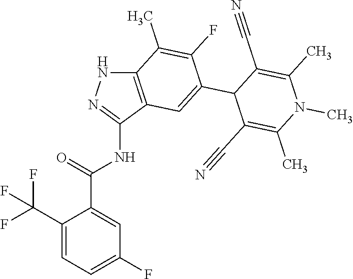 [SMILES: CC1=C(C#N)C(c2cc3c(NC(=O)c4cc(F)ccc4C(F)(F)F)n[nH]c3c(C)c2F)C(C#N)=C(C)N1C]

To a stirred solution of 4-(3-amino-6-fluoro-7-methyl-1H-indazol-5-yl)-1,2,6-trimethyl-1,4-dihydropyridine-3,5-dicarbonitrile (200 mg, 595 μmol) in DMA (5.8 mL) was added N,N-diisopropylethylamine (410 μl, 2.4 mmol), 5-fluoro-2-(trifluoromethyl)benzoic acid (186 mg, 892 μmol) and HATU (362 mg, 951 μmol). The mixture was stirred at 100° C. for 2 h in a microwave oven. An aqueous solution of sodium bicarbonate was added, the mixture was stirred for 15 minutes and the mixture was extracted with ethyl acetate. The organic phase was washed with saturated sodium chloride solution, dried (sodium sulfate), filtered and the solvent was removed in vacuum. Silicagel chromatography followed by aminophase-silicagel chromatography gave a solid that was triturated with a mixture of dichloromethane and hexane to give 107 mg (34% yield) of the title compound.

LC-MS (Method 1): Rₜ=1.11 min; MS (ESIpos): m/z=527 [M+H]⁺

¹H-NMR (400 MHz, DMSO-d6) δ[ppm]: 2.235 (16.00), 2.430 (4.75), 2.518 (0.98), 2.523 (0.66), 3.331 (10.12), 4.619 (2.09), 5.759 (2.04), 7.563 (0.40), 7.576 (1.57), 7.594 (1.22), 7.740 (0.68), 7.746 (0.71), 7.762 (0.71), 7.768 (0.66), 7.945 (0.69), 7.958 (0.73), 7.968 (0.67), 7.980 (0.62), 11.164 (2.00), 13.080 (1.73).

### Example 11

### 2-chloro-N-[5-(3,5-dicyano-1,2,6-trimethyl-1,4-dihydropyridin-4-yl)-6-fluoro-7-methyl-1H-indazol-3-yl]-6-fluorobenzamide

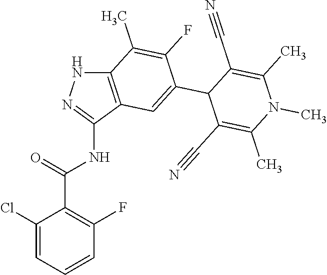 [SMILES: CC1=C(C#N)C(c2cc3c(NC(=O)c4c(F)cccc4Cl)n[nH]c3c(C)c2F)C(C#N)=C(C)N1C]

To a stirred solution of 4-(3-amino-6-fluoro-7-methyl-1H-indazol-5-yl)-1,2,6-trimethyl-1,4-dihydropyridine-3,5-dicarbonitrile (100 mg, 297 μmol), pyridine (72 μl, 890 μmol) and DMAP (3.63 mg, 29.7 μmol) in dichloromethane (15 ml) at r.t. was added 2-chloro-6-fluorobenzoyl chloride (86.1 mg, 446 μmol) and the mixture was stirred at r.t. for 2 h. Ethanol (0.2 mL) was added, the mixture was stirred at r.t. for 0.5 h and the solvents were removed in vacuum. Aminophase-silicagel chromatography followed by silicagel chromatography gave a solid that was triturated with dichloromethane to give 10.0 mg (90% purity, 6% yield) of the title compound.

LC-MS (Method 1): Rₜ=1.05 min; MS (ESIpos): m/z=493 [M+H]⁺

¹H-NMR (500 MHz, DMSO-d6) δ[ppm]: 2.257 (12.56), 2.448 (3.16), 2.521 (0.68), 3.059 (16.00), 3.236 (0.78), 4.594 (1.47), 7.353 (0.63), 7.428 (0.54), 7.443 (0.69), 7.535 (0.50), 7.550 (0.43), 7.605 (0.63), 7.617 (0.60), 10.988 (0.55), 12.856 (0.53).

### Example 12

### N-[5-(3,5-dicyano-1,2,6-trimethyl-1,4-dihydropyridin-4-yl)-6-fluoro-7-methyl-1H-indazol-3-yl]-1-ethyl-3-methyl-1H-pyrazole-4-carboxamide

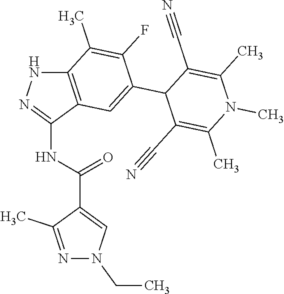

To a stirred solution of 4-(3-amino-6-fluoro-7-methyl-1H-indazol-5-yl)-1,2,6-trimethyl-1,4-dihydropyridine-3,5-dicarbonitrile (80.0 mg, 238 μmol), pyridine (58 μl, 710 μmol) and DMAP (2.91 mg, 23.8 μmol) in dichloromethane (12 ml) at r.t. was added 1-ethyl-3-methyl-1H-pyrazole-4-carbonyl chloride (61.6 mg, 357 μmol) and the mixture was stirred at r.t. for 16 h. A half-saturated solution of sodium bicarbonate was added and the mixture was extracted with dichloromethane. The organic phase was washed with saturated sodium chloride solution, dried (sodium sulfate) and the solvent was removed in vacuum. Aminophase-silicagel chromatography followed by silicagel chromatography followed by preparative reverse phase HPLC (Chromatex C18, 10 μm, 125×30 mm, gradient of water and acetonitrile containing 0.2% aqueous ammonia as additive) gave 21.0 mg (17% yield) of the title compound after lyophilisation.

LC-MS (Method 1): Rₜ=0.96 min; MS (ESIpos): m/z=472 [M+H]⁺

¹H-NMR (400 MHz, DMSO-d6) δ[ppm]: 1.376 (3.64), 1.395 (9.12), 1.413 (3.75), 2.232 (16.00), 2.394 (12.48), 2.406 (4.38), 2.409 (4.46), 2.518 (0.87), 2.523 (0.63), 3.332 (15.66), 4.072 (0.84), 4.090 (2.61), 4.108 (2.64), 4.126 (0.81), 4.604 (1.86), 7.596 (1.08), 7.613 (1.10), 8.468 (3.24), 10.311 (2.00), 12.923 (1.58).

### Example 13

### N-[5-(3,5-dicyano-1,2,6-trimethyl-1,4-dihydropyridin-4-yl)-6-fluoro-7-methyl-1H-indazol-3-yl]-4-[2-(pyrrolidin-1-yl)ethoxy]benzamide

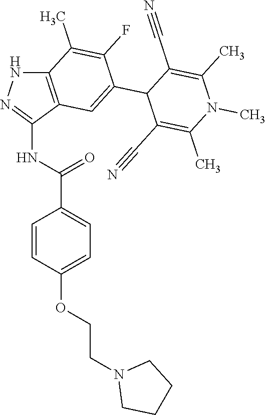 [SMILES: CC1=C(C#N)C(c2cc3c(NC(=O)c4ccc(OCCN5CCCC5)cc4)n[nH]c3c(C)c2F)C(C#N)=C(C)N1C]

To a stirred solution of 4-[2-(pyrrolidin-1-yl)ethoxy]benzoic acid-hydrogen chloride (1:1) (1.00 g, approx. 64% purity, 2.36 mmol) in dichloromethane (22 mL) was added DMF (9.1 μl, 120 μmol) and ethanedioyl dichloride (410 μl, 4.7 mmol) and the mixture was stirred at reflux for 2 h and then allowed to cool down to r.t. An aliquote of the crude reaction mixture (1.6 mL, approx. 0.17 mmol) was added to a stirred solution of 4-(3-amino-6-fluoro-7-methyl-1H-indazol-5-yl)-1,2,6-trimethyl-1,4-dihydropyridine-3,5-dicarbonitrile (28.0 mg, 83.2 μmol), pyridine (27 μl, 330 μmol) and DMAP (1.02 mg, 8.32 μmol) in dichloromethane (4.1 ml) and the mixture was stirred at r.t. for 16 h. A half-saturated solution of sodium bicarbonate was added and the mixture was extracted with dichloromethane. The organic phase was washed with saturated sodium chloride solution, dried (sodium sulfate) and the solvent was removed in vacuum. Aminophase-silicagel chromatography followed by preparative reverse phase HPLC (Chromatex C18, 10 μm, 125×30 mm, gradient of water and acetonitrile containing 0.2% aqueous ammonia as additive) gave 12.0 mg of the title compound after lyophilisation.

LC-MS (Method 2): Rₜ=1.14 min; MS (ESIneg): m/z=552 [M−H]⁻

¹H-NMR (400 MHz, DMSO-d6) δ[ppm]: 1.673 (1.49), 1.681 (2.17), 1.689 (4.42), 1.698 (2.11), 1.707 (1.48), 2.220 (16.00), 2.420 (4.63), 2.423 (4.72), 2.518 (2.48), 2.522 (2.79), 2.527 (3.82), 2.534 (1.99), 2.539 (1.79), 2.544 (1.48), 2.798 (1.52), 2.812 (3.40), 2.827 (1.59), 3.207 (10.21), 4.149 (1.49), 4.165 (3.04), 4.179 (1.41), 4.631 (1.91), 7.056 (3.04), 7.061 (0.94), 7.079 (3.04), 7.502 (1.17), 7.520 (1.19), 8.048 (2.99), 8.053 (0.96), 8.071 (2.68), 10.680 (1.94), 13.022 (1.26).

### Example 14

### N-[5-(3,5-dicyano-1,2,6-trimethyl-1,4-dihydropyridin-4-yl)-6-fluoro-7-methyl-1H-indazol-3-yl]-2-(6-methylpyridin-3-yl)acetamide

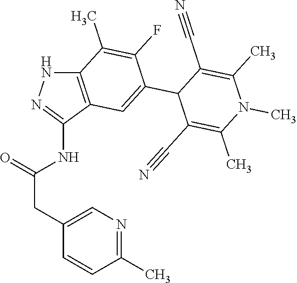 [SMILES: CC1=C(C#N)C(c2cc3c(NC(=O)Cc4ccc(C)nc4)n[nH]c3c(C)c2F)C(C#N)=C(C)N1C]

To a stirred solution of 4-(3-amino-6-fluoro-7-methyl-1H-indazol-5-yl)-1,2,6-trimethyl-1,4-dihydropyridine-3,5-dicarbonitrile (50.0 mg, 149 μmol) in DMF (1.5 mL) was added N,N-diisopropylethylamine (78 μl, 450 μmol), (6-methylpyridin-3-yl)acetic acid (27.0 mg, 178 μmol) and HATU (79.1 mg, 208 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography gave a solid that was triturated with dichloromethane to give 21.0 mg (30% yield) of the title compound.

LC-MS (Method 1): Rₜ=0.77 min; MS (ESIpos): m/z=470 [M+H]⁺

¹H-NMR (400 MHz, DMSO-d6) δ[ppm]: 2.187 (16.00), 2.231 (0.42), 2.383 (6.56), 2.449 (11.19), 2.518 (1.96), 2.523 (1.35), 3.145 (8.07), 3.720 (3.93), 4.543 (2.02), 5.759 (2.34), 7.224 (1.34), 7.245 (1.43), 7.453 (1.14), 7.471 (1.16), 7.657 (0.94), 7.672 (0.86), 8.434 (1.59), 10.771 (1.59), 12.922 (1.57).

### Example 15

### N-[5-(3,5-dicyano-1,2,6-trimethyl-1,4-dihydropyridin-4-yl)-6-fluoro-7-methyl-1H-indazol-3-yl]-1-(2-methoxyethyl)-3-(trifluoromethyl)-1H-pyrazole-4-carboxamide

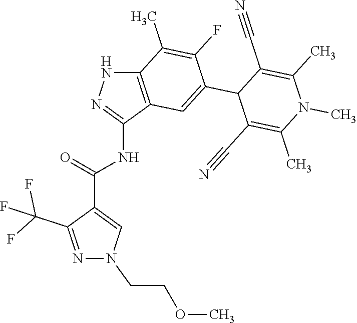 [SMILES: COCCn1cc(C(=O)Nc2n[nH]c3c(C)c(F)c(C4C(C#N)=C(C)N(C)C(C)=C4C#N)cc23)c(C(F)(F)F)n1]

To a stirred solution of 1-(2-methoxyethyl)-3-(trifluoromethyl)-1H-pyrazole-4-carboxylic acid (63.7 mg, 268 μmol) in DMA (2.7 mL) was added N,N-diisopropylethylamine (120 μl, 670 μmol), 4-(3-amino-6-fluoro-7-methyl-1H-indazol-5-yl)-1,2,6-trimethyl-1,4-dihydropyridine-3,5-dicarbonitrile (75.0 mg, 223 μmol) and HATU (119 mg, 312 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography gave a solid that was triturated with dichloromethane to give 27.0 mg (20% yield) of the title compound.

LC-MS (Method 1): Rₜ=1.06 min; MS (ESIpos): m/z=557 [M+H]⁺

¹H-NMR (400 MHz, DMSO-d6) δ[ppm]: 2.228 (16.00), 2.415 (4.35), 2.418 (4.45), 2.518 (0.98), 2.523 (0.67), 3.205 (9.65), 3.331 (13.71), 3.737 (1.33), 3.749 (2.07), 3.762 (1.43), 4.409 (1.22), 4.422 (1.89), 4.434 (1.12), 4.598 (2.02), 7.593 (0.97), 7.610 (0.98), 8.697 (1.08), 10.848 (1.43), 13.022 (1.41).

### Example 16

### N-[5-(3,5-dicyano-1,2,6-trimethyl-1,4-dihydropyridin-4-yl)-6-fluoro-7-methyl-1H-indazol-3-yl]-1-[2-(pyrrolidin-1-yl)ethyl]-3-(trifluoromethyl)-1H-pyrazole-4-carboxamide

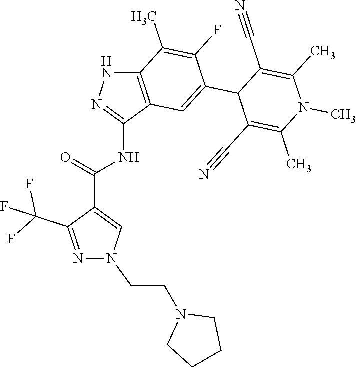 [SMILES: CC1=C(C#N)C(c2cc3c(NC(=O)c4cn(CCN5CCCC5)nc4C(F)(F)F)n[nH]c3c(C)c2F)C(C#N)=C(C)N1C]

To a stirred solution of 1-[2-(pyrrolidin-1-yl)ethyl]-3-(trifluoromethyl)-1H-pyrazole-4-carboxylic acid (371 mg, 20% purity, 268 μmol) in DMA (2.7 mL) was added N,N-diisopropylethylamine (120 μl, 670 μmol), 4-(3-amino-6-fluoro-7-methyl-1H-indazol-5-yl)-1,2,6-trimethyl-1,4-dihydropyridine-3,5-dicarbonitrile (75.0 mg, 223 μmol) and HATU (119 mg, 312 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography followed by silicagel chromatography gave 21.0 mg (14% yield) of the title compound.

LC-MS (Method 1): Rₜ=0.86 min; MS (ESIpos): m/z=596 [M+H]⁺

¹H-NMR (400 MHz, DMSO-d6) δ[ppm]: 1.691 (2.53), 2.228 (16.00), 2.417 (4.85), 2.878 (0.78), 3.205 (9.02), 4.344 (0.71), 4.359 (1.20), 4.594 (2.10), 5.758 (2.45), 7.600 (0.98), 7.617 (0.98), 8.713 (1.11), 10.839 (1.64), 13.024 (1.90).

### Example 17

### N-[5-(3,5-dicyano-1,2,6-trimethyl-1,4-dihydropyridin-4-yl)-6-fluoro-7-methyl-1H-indazol-3-yl]-1-(2-hydroxy-2-methylpropyl)-3-(trifluoromethyl)-1H-pyrazole-4-carboxamide

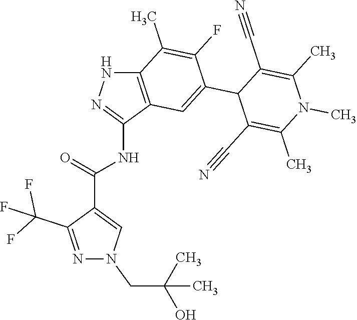 [SMILES: CC1=C(C#N)C(c2cc3c(NC(=O)c4cn(CC(C)(C)O)nc4C(F)(F)F)n[nH]c3c(C)c2F)C(C#N)=C(C)N1C]

To a stirred solution of 1-(2-hydroxy-2-methylpropyl)-3-(trifluoromethyl)-1H-pyrazole-4-carboxylic acid (67.5 mg, 268 μmol) in DMA (2.7 mL) was added N,N-diisopropylethylamine (120 μl, 670 μmol), 4-(3-amino-6-fluoro-7-methyl-1H-indazol-5-yl)-1,2,6-trimethyl-1,4-dihydropyridine-3,5-dicarbonitrile (75.0 mg, 223 μmol) and HATU (119 mg, 312 μmol). The mixture was stirred at 90° C. for 14 h. Further HATU (170 mg, 446 μmol) and N,N-diisopropylethylamine (80 μl, 446 μmol) was added and the mixture was stirred at r.t. for 4 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography gave a solid that was triturated with dichloromethane to give 20.0 mg (14 yield) of the title compound.

LC-MS (Method 1): Rₜ=1.04 min; MS (ESIpos): m/z=571 [M+H]⁺

¹H-NMR (400 MHz, DMSO-d6) δ[ppm]: 1.122 (13.38), 2.228 (16.00), 2.416 (4.58), 2.518 (1.24), 2.523 (0.83), 3.205 (9.26), 4.151 (3.72), 4.599 (2.04), 4.904 (3.36), 5.759 (0.94), 7.595 (0.93), 7.612 (0.94), 8.674 (0.98), 10.887 (1.49), 13.017 (1.57).

### Example 18

### N-[5-(3,5-dicyano-1,2,6-trimethyl-1,4-dihydropyridin-4-yl)-6-fluoro-7-methyl-1H-indazol-3-yl]-1-(2-hydroxy-2-methylpropyl)-5-(trifluoromethyl)-1H-pyrazole-4-carboxamide

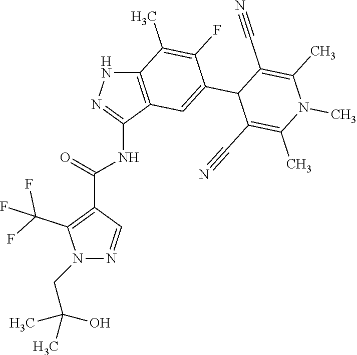 [SMILES: CC1=C(C#N)C(c2cc3c(NC(=O)c4cnn(CC(C)(C)O)c4C(F)(F)F)n[nH]c3c(C)c2F)C(C#N)=C(C)N1C]

To a stirred solution of 1-(2-hydroxy-2-methylpropyl)-5-(trifluoromethyl)-1H-pyrazole-4-carboxylic acid (64.3 mg, 42% purity, 107 μmol) in DMA (1.1 mL) was added N,N-diisopropylethylamine (78 μl, 450 μmol), 4-(3-amino-6-fluoro-7-methyl-1H-indazol-5-yl)-1,2,6-trimethyl-1,4-dihydropyridine-3,5-dicarbonitrile (30.0 mg, 89.2 μmol) and HATU (47.5 mg, 125 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography followed by silicagel chromatography gave 11.0 mg (21% yield) of the title compound.

LC-MS (Method 1): Rₜ=1.00 min; MS (ESIpos): m/z=571 [M+H]⁺

¹H-NMR (400 MHz, DMSO-d6) δ[ppm]: 1.135 (11.42), 2.084 (2.44), 2.228 (16.00), 2.417 (4.42), 2.518 (1.65), 2.523 (1.11), 3.203 (9.29), 4.266 (2.42), 4.609 (1.96), 4.803 (3.66), 5.759 (5.00), 7.541 (0.61), 7.559 (0.61), 8.229 (0.52), 10.910 (0.66), 13.044 (1.32).

### Example 19

### N-[5-(3,5-dicyano-1,2,6-trimethyl-1,4-dihydropyridin-4-yl)-6-fluoro-7-methyl-1H-indazol-3-yl]-1-[2-(pyrrolidin-1-yl)ethyl]-5-(trifluoromethyl)-1H-pyrazole-4-carboxamide

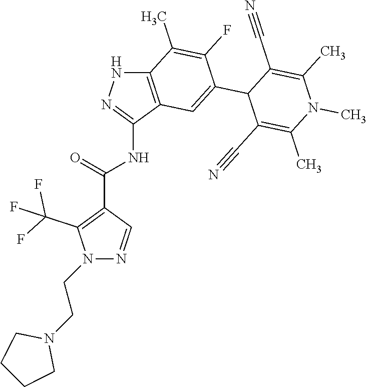 [SMILES: CC1=C(C#N)C(c2cc3c(NC(=O)c4cnn(CCN5CCCC5)c4C(F)(F)F)n[nH]c3c(C)c2F)C(C#N)=C(C)N1C]

To a stirred solution of 1-[2-(pyrrolidin-1-yl)ethyl]-5-(trifluoromethyl)-1H-pyrazole-4-carboxylic acid (60.6 mg, 49% purity, 107 μmol) in DMA (1.1 mL) was added N,N-diisopropylethylamine (78 μl, 450 μmol), 4-(3-amino-6-fluoro-7-methyl-1H-indazol-5-yl)-1,2,6-trimethyl-1,4-dihydropyridine-3,5-dicarbonitrile (30.0 mg, 89.2 μmol) and HATU (47.5 mg, 125 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography followed by silicagel chromatography gave 6.0 mg (11% yield) of the title compound.

LC-MS (Method 1): Rₜ=0.82 min; MS (ESIpos): m/z=596 [M+H]⁺

¹H-NMR (400 MHz, DMSO-d6) δ[ppm]: 1.154 (0.46), 1.172 (0.93), 1.190 (0.48), 1.667 (2.83), 1.987 (1.72), 2.227 (16.00), 2.326 (0.43), 2.415 (4.57), 2.518 (1.74), 2.522 (1.19), 2.668 (0.42), 2.872 (0.81), 3.203 (9.06), 4.017 (0.40), 4.434 (1.02), 4.608 (2.01), 7.538 (0.54), 7.554 (0.53), 8.183 (0.59), 10.980 (0.65), 13.048 (1.91).

### Example 20

### 2-chloro-N-[5-(3,5-dicyano-1,2,6-trimethyl-1,4-dihydropyridin-4-yl)-6-fluoro-7-methyl-1H-indazol-3-yl]-4-[2-(pyrrolidin-1-yl)ethoxy]benzamide

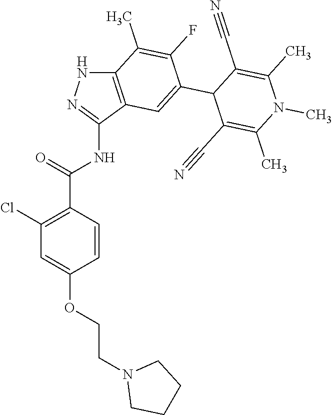 [SMILES: CC1=C(C#N)C(c2cc3c(NC(=O)c4ccc(OCCN5CCCC5)cc4Cl)n[nH]c3c(C)c2F)C(C#N)=C(C)N1C]

To a stirred solution of 2-chloro-4-[2-(pyrrolidin-1-yl)ethoxy]benzoic acid (96.2 mg, 357 μmol) in DMA (4.0 mL) was added N,N-diisopropylethylamine (210 μl, 1.2 mmol), 4-(3-amino-6-fluoro-7-methyl-1H-indazol-5-yl)-1,2,6-trimethyl-1,4-dihydropyridine-3,5-dicarbonitrile (100 mg, 297 μmol) and HATU (158 mg, 416 μmol). The mixture was stirred at 90° C. for 14 h. Water was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography followed by silicagel chromatography gave 46.0 mg (26% yield) of the title compound.

LC-MS (Method 1): Rₜ=0.81 min; MS (ESIpos): m/z=588 [M+H]⁺

¹H-NMR (400 MHz, DMSO-d6) δ[ppm]: 1.695 (3.31), 2.084 (1.86), 2.235 (16.00), 2.417 (3.28), 2.518 (2.03), 2.522 (1.91), 2.539 (1.91), 2.815 (0.96), 3.209 (8.53), 4.164 (1.10), 4.622 (2.01), 5.759 (2.25), 7.019 (0.43), 7.038 (0.43), 7.161 (0.76), 7.601 (0.88), 10.825 (0.65), 13.005 (0.99).

### Example 21

### (rac)-N-[5-(3,5-dicyano-1,2,6-trimethyl-1,4-dihydropyridin-4-yl)-6-fluoro-7-methyl-1H-indazol-3-yl]-2-(pyridin-3-yl)propanamide

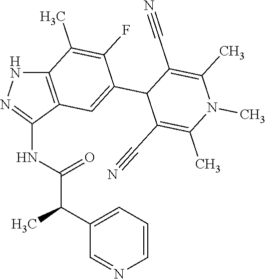 [SMILES: CC1=C(C#N)C(c2cc3c(NC(=O)[C@H](C)c4cccnc4)n[nH]c3c(C)c2F)C(C#N)=C(C)N1C]

To a stirred solution of (rac)-2-(pyridin-3-yl)propanoic acid (53.9 mg, 357 μmol) in DMA (4.0 mL) was added N,N-diisopropylethylamine (210 μl, 1.2 mmol), 4-(3-amino-6-fluoro-7-methyl-1H-indazol-5-yl)-1,2,6-trimethyl-1,4-dihydropyridine-3,5-dicarbonitrile (100 mg, 297 μmol) and HATU (158 mg, 416 μmol). The mixture was stirred at 90° C. for 14 h. Water was added, the mixture was stirred for 15 minutes and the mixture was extracted with ethyl acetate. The organic phase was washed with half-saturated sodium chloride solution, dried (sodium sulfate), filtered and the solvent was removed in vacuum. Aminophase-silicagel chromatography followed by silicagel chromatography gave a solid that was crystallized from dichloromethane to give 14.0 mg (10% yield) of the title compound.

LC-MS (Method 1): Rₜ=0.78 min; MS (ESIpos): m/z=470 [M+H]⁺

¹H-NMR (400 MHz, DMSO-d6) δ[ppm]: 1.486 (5.43), 1.503 (5.47), 2.201 (11.99), 2.239 (14.58), 2.377 (8.11), 2.518 (3.04), 2.523 (1.96), 3.200 (16.00), 3.218 (0.50), 3.995 (0.41), 4.012 (1.30), 4.030 (1.29), 4.548 (3.01), 5.759 (3.70), 7.380 (1.05), 7.392 (1.14), 7.400 (1.21), 7.412 (1.21), 7.425 (1.73), 7.443 (1.73), 7.823 (1.33), 7.843 (1.21), 8.480 (1.55), 8.484 (1.66), 8.492 (1.61), 8.496 (1.53), 8.647 (2.25), 8.652 (2.29), 10.757 (2.51), 12.925 (2.34).

### Example 22

### methyl 4-{[5-(3,5-dicyano-1,2,6-trimethyl-1,4-dihydropyridin-4-yl)-6-fluoro-7-methyl-1H-indazol-3-yl]carbamoyl}cubane-1-carboxylate

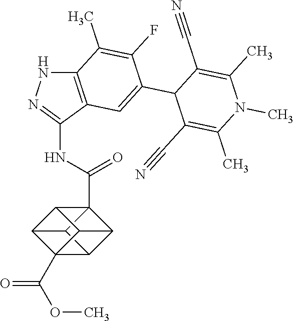 [SMILES: COC(=O)C12C3C4C1C1C2C3C41C(=O)Nc1n[nH]c2c(C)c(F)c(C3C(C#N)=C(C)N(C)C(C)=C3C#N)cc12]

To a stirred solution of 4-(methoxycarbonyl)cubane-1-carboxylic acid (230 mg, 1.11 mmol) in DMA (6.9 mL) was added N,N-diisopropylethylamine (520 μl, 3.0 mmol), 4-(3-amino-6-fluoro-7-methyl-1H-indazol-5-yl)-1,2,6-trimethyl-1,4-dihydropyridine-3,5-dicarbonitrile (250 mg, 743 μmol) and HATU (452 mg, 1.19 mmol). The mixture was stirred at 100° C. in a microwave oven for 0.5 h. An aqueous solution of sodium bicarbonate was added, the mixture was stirred for 15 minutes and the mixture was extracted with ethyl acetate. The organic phase was washed with saturated sodium chloride solution, dried (sodium sulfate), filtered and the solvent was removed in vacuum. Aminophase-silicagel chromatography gave 322 mg (82% yield) of the title compound.

LC-MS (Method 1): Rₜ=0.99 min; MS (ESIpos): m/z=525 [M+H]⁺

¹H-NMR (400 MHz, DMSO-d6) δ[ppm]: 2.085 (0.68), 2.228 (14.77), 2.392 (5.22), 2.518 (1.53), 2.523 (1.12), 3.218 (11.92), 3.643 (3.19), 4.190 (1.46), 4.313 (1.40), 4.599 (1.02), 5.759 (16.00), 7.521 (0.65), 7.539 (0.60), 10.399 (0.63), 12.949 (1.03).

### Example 23

### 4-(azetidine-1-carbonyl)-N-[5-(3,5-dicyano-1,2,6-trimethyl-1,4-dihydropyridin-4-yl)-6-fluoro-7-methyl-1H-indazol-3-yl]cubane-1-carboxamide

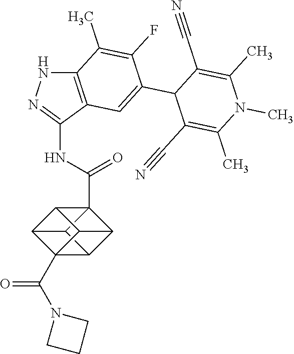 [SMILES: CC1=C(C#N)C(c2cc3c(NC(=O)C45C6C7C4C4C5C6C74C(=O)N4CCC4)n[nH]c3c(C)c2F)C(C#N)=C(C)N1C]

To a stirred solution of 4-{[5-(3,5-dicyano-1,2,6-trimethyl-1,4-dihydropyridin-4-yl)-6-fluoro-7-methyl-1H-indazol-3-yl]carbamoyl}cubane-1-carboxylic acid-hydrogen chloride (1:1) (150 mg, approx. 274 μmol) in DMA (2.5 mL) was added N,N-diisopropylethylamine (190 μl, 1.1 mmol), azetidine-hydrogen chloride (1:1) (38.5 mg, 411 μmol) and HATU (167 mg, 439 μmol). The mixture was stirred at r.t. for 16 h. An aqueous solution of sodium bicarbonate was added, the mixture was stirred for 15 minutes and the mixture was extracted with ethyl acetate. The organic phase was dried (sodium sulfate), filtered and the solvent was removed in vacuum. Silicagel chromatography followed by aminophase-silicagel chromatography gave a solid that was triturated with warm ethyl acetate to give 90.0 mg (57% yield) of the title compound.

LC-MS (Method 1): Rₜ=0.90 min; MS (ESIpos): m/z=550 [M+H]⁺

¹H-NMR (400 MHz, DMSO-d6) δ[ppm]: 0.831 (0.47), 0.852 (0.51), 0.858 (0.50), 1.154 (0.75), 1.172 (1.35), 1.190 (0.69), 1.242 (0.41), 1.988 (2.49), 2.228 (16.00), 2.393 (6.12), 3.332 (5.99), 3.883 (1.52), 4.017 (0.78), 4.035 (0.79), 4.053 (0.41), 4.152 (1.37), 4.200 (1.97), 4.279 (1.77), 4.602 (1.29), 7.524 (0.78), 7.538 (0.75), 10.354 (0.74), 12.942 (1.93).

### Example 24

### N-[5-(3,5-dicyano-1,2,6-trimethyl-1,4-dihydropyridin-4-yl)-6-fluoro-7-methyl-1H-indazol-3-yl]-4-(4-methylpiperazine-1-carbonyl)cubane-1-carboxamide

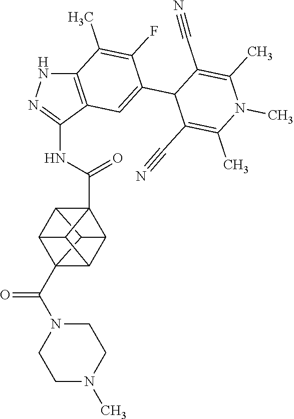 [SMILES: CC1=C(C#N)C(c2cc3c(NC(=O)C45C6C7C4C4C5C6C74C(=O)N4CCN(C)CC4)n[nH]c3c(C)c2F)C(C#N)=C(C)N1C]

To a stirred solution of 4-{[5-(3,5-dicyano-1,2,6-trimethyl-1,4-dihydropyridin-4-yl)-6-fluoro-7-methyl-1H-indazol-3-yl]carbamoyl}cubane-1-carboxylic acid-hydrogen chloride (1:1) (150 mg, approx. 274 μmol) in DMA (2.5 mL) was added N,N-diisopropylethylamine (190 μl, 1.1 mmol), 1-methylpiperazine (46 μl, 410 μmol) and HATU (167 mg, 439 μmol). The mixture was stirred at r.t. for 16 h. An aqueous solution of sodium bicarbonate was added, the mixture was stirred for 15 minutes and the mixture was extracted with ethyl acetate. The organic phase dried (sodium sulfate), filtered and the solvent was removed in vacuum. Silicagel chromatography gave a solid that was triturated with dichloromethane to give 90.0 mg (53% yield) of the title compound.

LC-MS (Method 1): Rₜ=0.90 min; MS (ESIpos): m/z=593 [M+H]⁺

¹H-NMR (400 MHz, DMSO-d6) δ[ppm]: 0.831 (0.55), 0.859 (0.40), 1.155 (0.79), 1.173 (1.69), 1.190 (0.85), 1.907 (0.50), 1.988 (2.78), 2.084 (0.64), 2.193 (4.10), 2.230 (12.01), 2.261 (0.99), 2.323 (0.51), 2.327 (0.68), 2.332 (0.69), 2.349 (0.80), 2.394 (4.24), 2.518 (1.18), 2.523 (0.78), 3.332 (16.00), 3.432 (0.82), 4.017 (0.65), 4.035 (0.63), 4.162 (1.04), 4.302 (0.99), 4.601 (0.80), 7.538 (0.45), 7.553 (0.41), 10.392 (0.44), 12.946 (0.82).

### Example 25

### N-[5-(3,5-dicyano-1,2,6-trimethyl-1,4-dihydropyridin-4-yl)-7-ethenyl-6-fluoro-1H-indazol-3-yl]-1-ethyl-3-methyl-1H-pyrazole-5-carboxamide

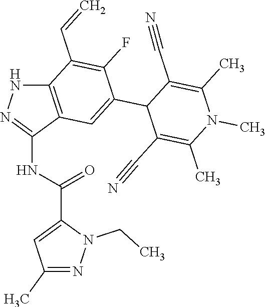 [SMILES: C=Cc1c(F)c(C2C(C#N)=C(C)N(C)C(C)=C2C#N)cc2c(NC(=O)c3cc(C)nn3CC)n[nH]c12]

To a stirred solution of N-[5-(3,5-dicyano-1,2,6-trimethyl-1,4-dihydropyridin-4-yl)-6-fluoro-7-iodo-1H-indazol-3-yl]-1-ethyl-3-methyl-1H-pyrazole-5-carboxamide (10.5 mg, 18.0 μmol) in 1-propanol (320 μl) was added pyridine-triethenylboroxin (1:1) (5.92 mg, 95% purity, 23.4 μmol), PdCl₂(PPh₃)₂ (1.89 mg, 2.70 μmol), triphenyl phosphine (710 μg, 2.7 μmol) and sodium bicarbonate solution (45 μl, 2.0 M, 90 μmol). The mixture was heated to reflux for 15 min. The mixture was concentrated in vacuum. Preparative TLC gave 4.50 mg (51% yield) of the title compound.

LC-MS (Method 1): Rₜ=1.05 min; MS (ESIpos): m/z=485 [M+H]⁺

¹H-NMR (400 MHz, DMSO-d6) δ[ppm]: 1.232 (0.91), 1.320 (2.94), 1.338 (7.06), 1.356 (2.93), 2.219 (9.61), 2.233 (16.00), 2.250 (0.58), 2.518 (1.52), 2.522 (1.08), 3.202 (10.25), 4.452 (0.61), 4.470 (1.89), 4.487 (1.84), 4.505 (0.58), 4.716 (1.81), 5.734 (1.21), 5.763 (1.26), 6.111 (0.80), 6.156 (0.87), 6.979 (1.71), 7.000 (1.03), 7.029 (1.02), 7.045 (1.00), 7.074 (0.84), 7.643 (1.04), 7.661 (1.04), 10.820 (1.46), 13.186 (1.26).

### Example 26

### N-[5-(3,5-dicyano-1,2,6-trimethyl-1,4-dihydropyridin-4-yl)-7-ethyl-6-fluoro-1H-indazol-3-yl]-2,6-difluorobenzamide

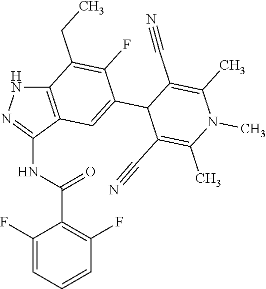 [SMILES: CCc1c(F)c(C2C(C#N)=C(C)N(C)C(C)=C2C#N)cc2c(NC(=O)c3c(F)cccc3F)n[nH]c12]

tert-butyl 3-amino-5-(3,5-dicyano-1,2,6-trimethyl-1,4-dihydropyridin-4-yl)-7-ethyl-6-fluoro-1H-indazole-1-carboxylate (50.0 mg, 85% purity, 94.3 μmol) and triethylamine (39 μl, 280 μmol), were dissolved in 2 mL of dichloromethane. Then 2,6-difluorobenzoyl chloride (22 mg, 0.12 mmol) was added at room temperature dropwise and the resulting mixture was stirred at this temperature for 6 h. Dichloromethane was added and the mixture was washed with brine, dried over anhydrous sodium sulfate and concentrated in vacuo. The residue was purified by prep-HPLC (Column: XBridge Prep C18 OBD Column 19×150 mm 5 μm; Mobile Phase A: Water (10 mmol/L NH4HCO3), Mobile Phase B: ACN; Flow rate: 20 mL/min; Gradient: 35% B to 60% B in 7 min; Detector: 254 nm, 220 nm) to give 6.1 mg (13% yield) of the product as an off-white solid.

LC-MS [Water(0.05% TFA)-Acetonitrile, 5% B]: Rₜ=2.38 min.

MS (ESIpos): m/z=491 (M+H)⁺.

¹H-NMR (400 MHz, DMSO-d6) δ[ppm]: 1.24 (t, 3H), 2.24 (s, 6H), 2.90 (q, 2H), 3.21 (s, 3H), 4.64 (s, 1H), 7.26-7.30 (m, 2H), 7.52-7.54 (m, 1H), 7.57-7.65 (m, 1H), 11.26 (s, 1H), 13.12 (s, 1H).

### Example 27

### N-[5-(3,5-dicyano-1,2,6-trimethyl-1,4-dihydropyridin-4-yl)-7-ethyl-6-fluoro-1H-indazol-3-yl]-2-ethylbenzamide

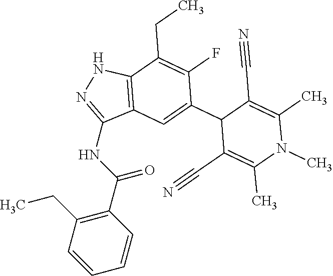 [SMILES: CCc1ccccc1C(=O)Nc1n[nH]c2c(CC)c(F)c(C3C(C#N)=C(C)N(C)C(C)=C3C#N)cc12]

tert-butyl 3-amino-5-(3,5-dicyano-1,2,6-trimethyl-1,4-dihydropyridin-4-yl)-7-ethyl-6-fluoro-1H-indazole-1-carboxylate (100 mg, 85% purity, 189 μmol), HATU (100 mg, 264 μmol), and N,N-diisopropylethylamine (99 μl, 570 μmol), were dissolved in 3 mL of the N,N-dimethylformamide, the resulting mixture was stirred at room temperature for 15 min, then 2-ethylbenzoic acid (34.0 mg, 226 μmol) was added and the resulting mixture was stirred at 90° C. for 16 h. The resulting mixture was diluted by addition of water and extracted with ethyl acetate. The combined organic phases were washed with water and brine, dried over anhydrous sodium sulfate and concentrated in vacuo and the residue was purified by prep-HPLC (Column: XBridge Prep C18 OBD Column 19×150 mm 5 μm; Mobile Phase A: Water (10 mmol/L NH4HCO3), Mobile Phase B: ACN; Flow rate: 20 mL/min; Gradient: 25% B to 65% B in 7 min; Detector: 254 nm, 220 nm) to give 36.2 mg (38% yield) of the product as a light yellow solid.

LC-MS [Water(0.05% TFA)-Acetonitrile, 5% B]: Rₜ=1.46 min.

MS (ESIpos): m/z=483 (M+H)⁺.

¹H-NMR (400 MHz, DMSO-d6) δ[ppm]: 1.19-1.26 (m, 6H), 2.27 (s, 6H), 2.79-2.92 (m, 4H), 3.20 (s, 3H), 4.66 (s, 1H), 7.29-7.36 (m, 2H), 7.43-7.46 (m, 1H), 7.51-7.58 (m, 2H), 10.77 (s, 1H), 13.01 (s, 1H).

### Example 28

### N-[5-(3,5-dicyano-1,2,6-trimethyl-1,4-dihydropyridin-4-yl)-7-ethyl-6-fluoro-1H-indazol-3-yl]-1-(propan-2-yl)-1H-pyrazole-5-carboxamide

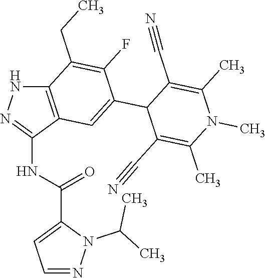 [SMILES: CCc1c(F)c(C2C(C#N)=C(C)N(C)C(C)=C2C#N)cc2c(NC(=O)c3ccnn3C(C)C)n[nH]c12]

1-(propan-2-yl)-1H-pyrazole-5-carboxylic acid (37.8 mg, 245 μmol), HATU (108 mg, 283 μmol), and N,N-diisopropylethylamine (99 μl, 570 μmol), were dissolved in 3 mL of the N,N-dimethylformamide and the resulting mixture was stirred at room temperature for 15 min, then tert-butyl 3-amino-5-(3,5-dicyano-1,2,6-trimethyl-1,4-dihydropyridin-4-yl)-7-ethyl-6-fluoro-1H-indazole-1-carboxylate (100 mg, 85% purity, 189 μmol) was added and the resulting mixture was stirred at 90° C. for 16 h. The resulting mixture was diluted by addition of water and extracted with ethyl acetate, the combined organic phases was washed with water and brine, dried over anhydrous sodium sulfate and concentrated in vacuo. The residue was purified by prep-HPLC (Column: XBridge Prep C18 OBD Column 19×150 mm, 5 μm; Mobile Phase A: Water (10 mmol/L NH4HCO3), Mobile Phase B: ACN; Flow rate: 20 mL/min; Gradient: 25% B to 65% B in 7 min; Detector: 254 nm, 220 nm) to give 39.9 mg (43% yield) of the product as a light yellow solid.

LC-MS [Water(0.05% TFA)-Acetonitrile, 5% B]: Rₜ=1.38 min.

MS (ESIpos): m/z=487 (M+H)⁺.

¹H-NMR (400 MHz, DMSO-d6) δ[ppm]: 1.24 (t, 3H), 1.44 (d, 6H), 2.23 (s, 6H), 2.90 (q, 2H), 3.20 (s, 3H), 4.66 (s, 1H), 5.50-5.56 (m, 1H), 7.16 (s, 1H), 7.53-7.55 (m, 1H), 7.59 (s, 1H), 10.85 (s, 1H), 13.12 (s, 1H).

### Example 29

### 4-chloro-N-[5-(3,5-dicyano-1,2,6-trimethyl-1,4-dihydropyridin-4-yl)-7-ethyl-6-fluoro-1H-indazol-3-yl]-1-methyl-1H-pyrazole-5-carboxamide

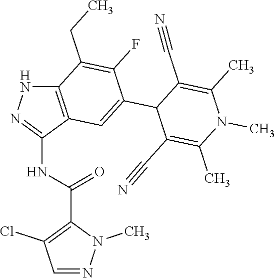 [SMILES: CCc1c(F)c(C2C(C#N)=C(C)N(C)C(C)=C2C#N)cc2c(NC(=O)c3c(Cl)cnn3C)n[nH]c12]

4-chloro-1-methyl-1H-pyrazole-5-carboxylic acid (39.4 mg, 245 μmol), HATU (108 mg, 283 μmol), and N,N-diisopropylethylamine (99 μl, 570 μmol), were dissolved in 3 mL of the N,N-dimethylformamide, the resulting mixture was stirred at room temperature for 15 min, then tert-butyl 3-amino-5-(3,5-dicyano-1,2,6-trimethyl-1,4-dihydropyridin-4-yl)-7-ethyl-6-fluoro-1H-indazole-1-carboxylate (100 mg, 85% purity, 189 μmol), was added and the resulting mixture was stirred at 90° C. for 16 h. The resulting mixture was diluted by addition of water and extracted with ethyl acetate. The combined organic phases were washed with water and brine, dried over anhydrous sodium sulfate and concentrated in vacuo. The residue was purified by prep-HPLC (Column: XBridge Prep C18 OBD Column 19×150 mm, 5 μm; Mobile Phase A: Water (10 mmol/L NH4HCO3), Mobile Phase B: ACN; Flow rate: 20 mL/min; Gradient: 25% B to 65% B in 7 min; Detector: 254 nm, 220 nm) to give 41.5 mg (44% yield) of the product as a light yellow solid.

LC-MS [Water(0.05% TFA)-Acetonitrile, 5% B)]: Rₜ=2.34 min.

MS (ESIpos): m/z=493 (M+H)⁺.

¹H-NMR (400 MHz, DMSO-d6) δ[ppm]: 1.23 (t, 3H), 2.24 (s, 6H), 2.90 (q, 2H), 3.21 (s, 3H), 3.99 (s, 3H), 4.66 (s, 1H), 7.58-7.60 (m, 1H), 7.70 (s, 1H), 11.02 (s, 1H), 13.15 (s, 1H).

### Example 30

### N-[5-(3,5-dicyano-1,2,6-trimethyl-1,4-dihydropyridin-4-yl)-7-ethyl-6-fluoro-1H-indazol-3-yl]-1-ethyl-3-methyl-1H-pyrazole-5-carboxamide

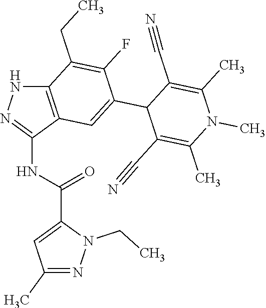 [SMILES: CCc1c(F)c(C2C(C#N)=C(C)N(C)C(C)=C2C#N)cc2c(NC(=O)c3cc(C)nn3CC)n[nH]c12]

1-ethyl-3-methyl-1H-pyrazole-5-carboxylic acid (37.8 mg, 245 μmol), HATU (108 mg, 283 μmol), and N,N-diisopropylethylamine (99 μl, 570 μmol) were dissolved in 3 mL of the N,N-dimethylformamide and the resulting mixture was stirred at room temperature for 15 min. Then tert-butyl 3-amino-5-(3,5-dicyano-1,2,6-trimethyl-1,4-dihydropyridin-4-yl)-7-ethyl-6-fluoro-1H-indazole-1-carboxylate (100 mg, 85% purity, 189 μmol), was added and the resulting mixture was stirred at 90° C. for 16 h. The resulting mixture was diluted by addition of water and extracted with ethyl acetate. The combined organic phases were washed with water and brine, dried over anhydrous sodium sulfate and concentrated in vacuo. The residue was purified by prep-HPLC (Column: XBridge Prep C18 OBD Column 19×150 mm, 5 μm; Mobile Phase A: Water (10 mmol/L NH4HCO3), Mobile Phase B: ACN; Flow rate: 20 mL/min; Gradient: 25% B to 65% B in 7 min; Detector: 254 nm, 220 nm) to give 39.2 mg (42% yield) of the product as a light yellow solid.

LC-MS [Water(0.05% TFA)-Acetonitrile, 5% B]: Rₜ=1.34 min.

MS (ESIpos): m/z=487 (M+H)⁺.

¹H-NMR (400 MHz, DMSO-d6) δ[ppm]: 1.23 (t, 3H), 1.34 (t, 3H), 2.22-2.23 (m, 9H), 2.90 (q, 2H), 3.20 (s, 3H), 4.48 (q, 2H), 4.66 (s, 1H), 6.97 (s, 1H), 7.50-7.52 (m, 1H), 10.76 (s, 1H), 13.10 (s, 1H).

### Example 31

### 2-chloro-N-[5-(3,5-dicyano-1,2,6-trimethyl-1,4-dihydropyridin-4-yl)-7-ethyl-6-fluoro-1H-indazol-3-yl]benzamide

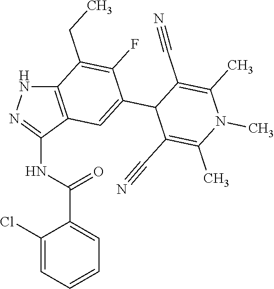 [SMILES: CCc1c(F)c(C2C(C#N)=C(C)N(C)C(C)=C2C#N)cc2c(NC(=O)c3ccccc3Cl)n[nH]c12]

2-Chlorobenzoic acid, (38.4 mg, 245 μmol), HATU (108 mg, 283 μmol), N,N-diisopropylethylamine, (99 μl, 570 μmol), and tert-Butyl-3-amino-5-(3,5-dicyano-1,2,6-trimethyl-1,4-dihydropyridin-4-yl)-7-ethyl-6-fluoro-1H-indazole-1-carboxylate (100 mg, 85% purity, 189 μmol), were dissolved in 3 mL of N,N-dimethylformamide and the resulting mixture was stirred at 90° C. for 16 hours. After cooled to room temperature, water was added and the mixture was extracted with ethyl acetate. The combined organic layers were washed with brine and dried over anhydrous sodium sulfate. The solvent was removed in vacuo and the residue was purified by prep-HPLC (Column: XBridge Prep C18 OBD Column 19×150 mm, 5 μm; Mobile Phase A: Water (10 mmol/L NH4HCO3), Mobile Phase B: ACN; Flow rate: 20 mL/min; Gradient: 25% B to 65% B in 7 min; Detector: 254 nm, 220 nm) to give 34.9 mg (37% yield) of the product as a light yellow solid.

LC-MS [Water(0.05% TFA)-Acetonitrile, 5% B]: Rₜ=1.38 min.

MS (ESIpos): m/z=489 (M+H)⁺.

¹H NMR (400 MHz, DMSO-d6) δ[ppm]: 1.24 (t, 3H), 2.24 (s, 6H), 2.90 (q, 2H), 3.21 (s, 3H), 4.65 (s, 1H), 7.46-7.55 (m, 2H), 7.59-7.66 (m, 3H), 10.96 (s, 1H), 13.05 (s, 1H).

### Example 32

### 2-chloro-N-[5-(3,5-dicyano-1,2,6-trimethyl-1,4-dihydropyridin-4-yl)-6-fluoro-7-(trifluoromethyl)-1H-indazol-3-yl]benzamide

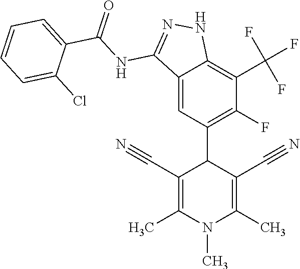 [SMILES: CC1=C(C#N)C(c2cc3c(NC(=O)c4ccccc4Cl)n[nH]c3c(C(F)(F)F)c2F)C(C#N)=C(C)N1C]

4-[3-amino-6-fluoro-7-(trifluoromethyl)-1H-indazol-5-yl]-1,2,6-trimethyl-1,4-dihydropyridine-3,5-dicarbonitrile (800 mg, 65% purity, 1.33 mmol), and triethylamine (560 μl, 4.0 mmol), were dissolved in 52 mL of 1,4-dioxane. Then 2-chlorobenzoyl chloride (350 mg, 2.00 mmol) was added at room temperature dropwise and the resulting mixture was stirred at this temperature for 23 h. The solvent was removed in vacuo and the residue was diluted with dichloromethane, the resulting mixture was washed with water and brine, dried over anhydrous sodium sulfate and concentrated in vacuo. The residue was purified by silica gel column chromatography followed by purification by prep-HPLC (Column: Atlantis® Prep OBD™ 5 μm 19*150 mm, Mobile Phase A: Water (containing 0.1% TFA), Mobile Phase B: ACN; Flow rate: 20 mL/min; Gradient: 45% B to 68% B in 8 min; 254 nm, 220 nm) to give 390 mg (50% yield) of the product as a light yellow solid.

LC-MS [Water(0.1% FA)-Acetonitrile, 10% B]: Rₜ=2.97 min.

MS (ESIpos): m/z=529 (M+H)⁺.

¹H-NMR (400 MHz, DMSO-d6) δ[ppm]: 2.26 (s, 6H), 3.23 (s, 3H), 4.84 (s, 1H), 7.47-7.56 (m, 2H), 7.60-7.62 (m, 1H), 7.69-7.71 (m, 1H), 8.18 (d, 1H), 11.26 (s, 1H), 13.38 (s, 1H).

### Example 33

### N-[5-(3,5-dicyano-1,2,6-trimethyl-1,4-dihydropyridin-4-yl)-6-fluoro-7-(trifluoromethyl)-1H-indazol-3-yl]-1-ethyl-3-methyl-1H-pyrazole-5-carboxamide

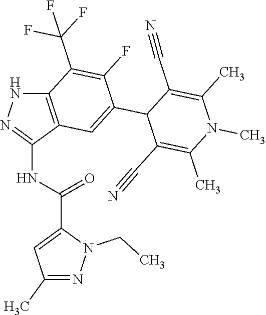 [SMILES: CCn1nc(C)cc1C(=O)Nc1n[nH]c2c(C(F)(F)F)c(F)c(C3C(C#N)=C(C)N(C)C(C)=C3C#N)cc12]

1-ethyl-3-methyl-1H-pyrazole-5-carboxylic acid (39.5 mg, 256 μmol) was dissolved in dichloromethane (10 mL), then ethanedioyl dichloride (150 μl, 2.0 M, 310 μmol) and DMF (2.0 μl, 26 μmol) were added and the resulting mixture was stirred at room temperature for 3 h. The solvent was removed in vacuo and the residue was redissolved in 1,4-dioxane. This solution was added dropwise to a solution of 4-[3-amino-6-fluoro-7-(trifluoromethyl)-1H-indazol-5-yl]-1,2,6-trimethyl-1,4-dihydropyridine-3,5-dicarbonitrile (100 mg, 256 μmol) and triethylamine (110 μl, 770 μmol) in 1,4-dioxane (10 mL) at room temperature. The resulting mixture was stirred at this temperature for 18 h. The solvent was removed in vacuo and the residue was diluted with dichloromethane. The resulting mixture was washed with water and brine, dried over anhydrous sodium sulfate and concentrated in vacuo. The residue was purified by silica gel chromatography (ethyl acetate/hexane=5:1) followed by purification by prep-HPLC (Column: Atlantis® Prep OBD™ 5 μm, 19*150 mm, Mobile Phase A: Water (containing 0.1 TFA), Mobile Phase B: ACN; Flow rate: 20 mL/min; Gradient: 40% B to 63% B in 8 min; 254 nm, 220 nm) to give 44.3 mg (33% yield) of the product as an off-white solid.

LC-MS [Water(0.1% FA)-Acetonitrile, 10% B]: Rₜ=1.39 min.

MS (ESIpos): m/z=527 (M+H)⁺.

¹H-NMR (400 MHz, DMSO-d6) δ[ppm]: 1.35 (t, 3H), 2.23 (s, 3H), 2.26 (s, 6H), 3.22 (s, 3H), 4.47-4.52 (q, 2H), 4.86 (s, 1H), 7.01 (s, 1H), 8.12 (d, 1H), 11.01 (s, 1H), 13.43 (s, 1H).

### Reference Example 1

### N-[5-(3,5-dicyano-2,6-dimethyl-1,4-dihydropyridin-4-yl)-1H-indazol-3-yl]benzamide

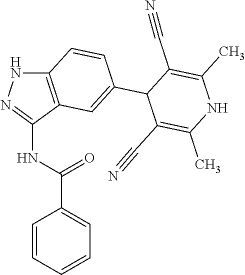 [SMILES: CC1=C(C#N)C(c2ccc3[nH]nc(NC(=O)c4ccccc4)c3c2)C(C#N)=C(C)N1]

Reference Example 1 was prepared as described in WO 2008/071451 A1, following the general procedures described therein.

### EXPERIMENTAL SECTION—BIOLOGICAL ASSAYS

The following assays can be used to illustrate the commercial utility of the compounds according to the present invention.

Examples were tested in selected biological assays one or more times. When tested more than once, data are reported as either average values or as median values or single individual measurements, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.
Individual measurements are shown when median or average values cannot be computed.

Examples were synthesized one or more times. When synthesized more than once, data from biological assays represent average values calculated utilizing data sets obtained from testing of one or more synthetic batch.

The in vitro activity of the compounds of the present invention can be demonstrated in the following assays:

AMPK Low ATP Assay

AMPK inhibitory activity of compounds of the present invention at an ATP concentration of 10 μM is quantified employing the TR-FRET-based AMPK activity inhibition assay as described in the following paragraphs.

Recombinant fusion protein of N-terminal Glutathion-S-Transferase (GST) and full-length human AMPKα2 [1-552(end) amino acids of accession number NP_006243.2] co-expressed with GST-PRKAB1 [1-270(end) amino acids of

accession number NP_006244.2] and PRKAG1 [1-331(end) amino acids of accession number NP_002724.1] using a baculovirus expression system, purified as GST-AMPKα2/ß1/γ1 complex by using glutathione sepharose chromatography, activated with His-tagged CaMKK1 and subsequently purified by using glutathione sepharose chromatography, was purchased from Carna Biosciences (product number 02-114) and used as kinase. As substrate for the kinase reaction biotinylated peptide biotin-Ahx-HMRSAMSFAEPG (C-terminus in amide form) is used which can be purchased e.g. form the company Biosyntan (Berlin-Buch, Germany).

For the assay 50 nl of a 100 fold concentrated solution of the test compound in DMSO is pipetted into either a black low volume 384 well microtiter plate or a black 1536 well microtiter plate (both Greiner Bio-One, Frickenhausen, Germany), 2 μl of a solution of GST-AMPKα2/ß1/γ1 in aqueous assay buffer [50 mM Hepes pH 7.5, 10 mM MgCl₂, 5 mM β-glycerophosphate, 2.5 mM dithiothreitol (DTT), 0.5 mM EGTA, 0.01% (w/v) bovine γ-globulin (Sigma-Aldrich, #G5009), 0.01% (v/v) Triton X-100 (Sigma-Aldrich, #T9284)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.67 μM=>final conc. in the 5 μl assay volume is 10 μM), adenosine-mono-phosphate (AMP, 3.33 μM=>final conc. in the 5 μl assay volume is 2 μM), and substrate (0.83 μM=>final conc. in the 5 μl assay volume is 0.5 μM) in assay buffer and the resulting mixture is incubated for a reaction time of 90 min at 22° C. The concentration of GST-AMPK α2/ß1/γ1 is adjusted depending of the activity of the enzyme lot and is chosen appropriate to have the assay in the linear range, a typical concentration was 0.05 nM. The reaction is stopped by the addition of 3 μl of a solution of TR-FRET detection reagents (0.2 μM streptavidine-XL665 [Cisbio Bioassays, Codolet, France] and 3.33 nM anti-phosho-Serine antibody [Merck Millipore, “STK antibody”, cat. #35-002] and 3.33 nM anti mouse IgG-Tb cryptate, a Terbium-cryptate labelled anti-mouse IgG antibody [Cisbio Bioassays, Codolet, France] in an aqueous EDTA-solution (166.7 mM EDTA, 0.06 (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XL665. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

AMPK High ATP Assay

AMPK inhibitory activity of compounds of the present invention at an ATP concentration of 1 mM was quantified employing the TR-FRET-based AMPK activity inhibition assay as described in the following paragraphs.

Recombinant fusion protein of N-terminal Glutathion-S-Transferase (GST) and full-length human AMPKα2 [1-552(end) amino acids of accession number NP_006243.2] co-expressed with GST-PRKAB1 [1-270(end) amino acids of accession number NP_006244.2] and PRKAG1 [1-331(end) amino acids of accession number NP_002724.1] using a baculovirus expression system, purified as GST-AMPKα2/ß1/γ1 complex by using glutathione sepharose chromatography, activated with His-tagged CaMKK1 and subsequently purified by using glutathione sepharose chromatography, was purchased from Carna Biosciences (product number 02-114) and used as kinase. As substrate for the kinase reaction biotinylated peptide biotin-Ahx-HMRSAMSFAEPG (C-terminus in amide form) was used which can be purchased e.g. form the company Biosyntan (Berlin-Buch, Germany). For the assay 50 nl of a 100 fold concentrated solution of the test compound in DMSO was pipetted into either a black low volume 384 well microtiter plate or a black 1536 well microtiter plate (both Greiner Bio-One, Frickenhausen, Germany), 2 μl of a solution of GST-AMPKα2/ß1/γ1 in aqueous assay buffer [50 mM Hepes pH 7.5, 10 mM MgCl₂, 5 mM β-glycerophosphate, 2.5 mM dithiothreitol (DTT), 0.5 mM EGTA, 0.01% (w/v) bovine γ-globulin (Sigma-Aldrich, #G5009), 0.01% (v/v) Triton X-100 (Sigma-Aldrich, #T9284)] were added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 1.67 mM=>final conc. in the 5 μl assay volume is 1 mM), adenosine-mono-phosphate (AMP, 3.33 μM=>final conc. in the 5 μl assay volume is 2 μM), and substrate (0.83 μM=>final conc. in the 5 μl assay volume is 0.5 μM) in assay buffer and the resulting mixture was incubated for a reaction time of 30 min at 22° C. The concentration of GST-AMPK α2/ß1/γ1 was adjusted depending of the activity of the enzyme lot and was chosen appropriate to have the assay in the linear range, a typical concentration was 0.08 nM. The reaction was stopped by the addition of 3 μl of a solution of TR-FRET detection reagents (0.2 μM streptavidine-XL665 [Cisbio Bioassays, Codolet, France] and 3.33 nM anti-phosho-Serine antibody [Merck Millipore, “STK antibody”, cat. #35-002] and 3.33 nM anti mouse IgG-Tb cryptate, a Terbium-cryptate labelled anti-mouse IgG antibody [Cisbio Bioassays, Codolet, France] in an aqueous EDTA-solution (166.7 mM EDTA, 0.06 (w/v) bovine serum albumin in 50 mM HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Tb-cryptate to the streptavidine-XL665. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0 inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

Enzyme Selectivity Profiling

Aurora-A (h)

Aurora-A (h) kinase activity is determined at Eurofins according to the following procedure:

Aurora-A (h) is incubated with 8 mM MOPS pH 7.0, 0.2 mM EDTA, 200 μM LRRASLG (Kemptide), 10 mM Magnesium acetate and [9-33P]-ATP (specific activity and concentration as required). The reaction is initiated by the addition of the Mg/ATP mix. After incubation for 40 minutes at room temperature, the reaction is stopped by the addition of phosphoric acid to a concentration of 0.5%. 10 μL of the reaction is then spotted onto a P30 filtermat and washed four times for 4 minutes in 0.425% phosphoric acid and once in methanol prior to drying and scintillation counting.

Cellular Mechanistic Assays

Phospho-ACC (Ser79) HTRF in COLO 320DM and IMR-32 Cells

This assay determines the levels of phospho-ACC in cell lysates only when phosphorylated on Serine 79. Phospho-ACC is detected in a sandwich HTRF assay with an anti-total ACC antibody labeled with d2 and an anti-phospho ACC antibody labeled with cryptate (Phospho-ACC (Ser79) Cellular Assay Kit, Cisbio).

On day 1, the cells (COLO 320DM or IMR-32) are seeded (25,000 cells in 12 μl/well) in a 384-well-SmallVolume-plate (Greiner Bio One #784075) in medium without glucose (DMEM, Biochrom #F0405) containing 10% FBS and 5 mM 2DG (2-Deoxy-D-glucose, Sigma #D8375-100G). The cells are then treated with different compounds or DMSO (added with the HP Dispenser) and incubated for 1 h at 37° C. Following incubation, the cells are lysed in 4 μl of lysis buffer for 1 h on ice. Finally, 4 μl of antibody solution (containing equal amounts of total ACC and phospho-ACC) is added and the samples are incubated overnight at 4° C. On the 2nd day, the plate is read on PHERAstar FS (BMG Labtech).

IC₅₀s were calculated using the DRC Master Spreadsheet (Bella software) and setting DMSO-treated cells as the minimum inhibition (C0) and Staurosporine-treated cells (1 μM of Staurosporine) as the maximum inhibition (Ci).

Proliferation Assays

Proliferation Assays Using Myc-Dependent Cells

For proliferation studies, cells are plated in cell culture media at a density of 800-1600 cells/25 μL/well in 384-well black plates (Corning #3571). Sister wells are plated in a separate plate for time zero determination and all plates were incubated overnight at 37° C.

On the next day, the test compounds are added in serial dilutions using the HP D300 Digital Dispenser and incubated at 37° C. for 72 h (COLO 320DM, LS-174T, COLO 201, Ramos, SNU-16, SU-DHL-10, OCI-LY7 and JJN-3) or 144 h (IMR-32, IMR-5/75 and SK-N-F1). The time zero plate is measured by adding 25 uL/well of CellTiter-Fluor solution (Promega, #G6080) followed by incubation for 30 minutes at 37° C. and measurement of fluorescence on PHERAStar (fluorometer 400 nmEX/505 nmEM, Gain:300). After 72 h/144 h incubation, the plates are measured as described above.

Background values measured with “medium only” are subtracted from all other values. Control wells, containing cells with culture medium and DMSO, are used to determine the control cell growth at 72 h/144 h compared to the initial number of cells (time zero value). To distinguish between cell growth inhibition and cell kill, the luminescence values are corrected after 72 h/144 h for the mean luminescence observed for the time zero wells at the day of drug addition (time zero value). IC₅₀s, defined as the drug concentration that corresponds to a reduction of cellular growth by 50% when compared with values of DMSO control cells, are calculated using the DRC Master Spreadsheet (Bella software).

Proliferation Assays Using Prostate Cancer Cells

For proliferation studies with prostate cancer cells, the cells are plated in RPMI 1640 medium without phenol red and supplemented with 10% charcoal-stripped FCS, at a density of 800-1000 cells/20 μL/well in 384-well black plates (Corning #3571). Sister wells are plated in a separate plate for time zero determination and all plates were incubated overnight at 37° C. On the next day, R1881 (final concentration: 0.1, 1 or 10 nM) are added, followed by addition of the test compounds in serial dilutions using the HP D300 Digital Dispenser and incubated at 37° C. for 144 h. The time zero plate is measured by adding 25 uL/well of CellTiter-Fluor solution (Promega, #G6080) followed by incubation for 30 minutes at 37° C. and measurement of fluorescence on PHERAStar (fluorometer 400 nmEX/505 nmEM, Gain:300). After 144 h incubation, the plates are measured as described above.

Background values measured with “medium only” are subtracted from all other values. Control wells, containing cells with culture medium, DMSO and R1881, are used to determine the control cell growth at 144 h compared to cells treated with DMSO, but without R1881. IC₅₀s, defined as the drug concentration that corresponds to a reduction of cellular growth by 50% when compared with values of DMSO+R1881 control cells, are calculated using the DRC Master Spreadsheet (Bella software).

Caco-2 Permeation Assay

Caco-2 cells (purchased from DSMZ Braunschweig, Germany) are seeded at a density of 4.5×10⁴ cell per well on 24 well insert plates, 0.4 μm pore size, and grown for 15 days in DMEM medium supplemented with 10% fetal bovine serum, 1% GlutaMAX (100×, GIBCO), 100 U/ml penicillin, 100 μg/ml streptomycin (GIBCO) and 1% non essential amino acids (100×). Cells are maintained at 37° C. in a humified 5% CO₂ atmosphere. Medium is changed every 2-3 day. Before running the permeation assay, the culture medium is replaced by a FCS-free hepes-carbonate transport puffer (pH 7.2) For assessment of monolayer integrity the transepithelial electrical resistance (TEER) is measured. Test compounds are predissolved in DMSO and added either to the apical or basolateral compartment in final concentration of 2 μM. Before and after 2 h incubation at 37° C. samples are taken from both compartments. Analysis of compound content is done after precipitation with methanol by LC/MS/MS analysis. Permeability (Papp) is calculated in the apical to basolateral (A→B) and basolateral to apical (B→A) directions. The apparent permeability is calculated using following equation:

Pₐₚₚ=(Vᵣ/Pₒ)(1/S)(P₂/t)

Where Vᵣ is the volume of medium in the receiver chamber, Pₒ is the measured peak area of the test drug in the donor chamber at t=o, S the surface area of the monolayer, P₂ is the measured peak area of the test drug in the acceptor chamber after 2 h of incubation, and t is the incubation time. The efflux ratio basolateral (B) to apical (A) is calculated by dividing the Pₐₚₚ B-A by the Pₐₚₚ A-B. In addition the compound recovery is calculated. As assay control reference compounds are analyzed in parallel.

Results:

Table 2 shows the results of the inhibition in the AMPK high ATP assay:

| Example | AMPK high ATP assay |
|---|---|
| No | IC₅₀ [mol/l] (median) |
| 1 | 6.26E−8 |
| 2 | 1.30E−8 |
| 3 | 5.43E−8 |
| 4 | 2.70E−9 |
| 5 | 3.74E−9 |
| 6 | 3.65E−9 |
| 7 | 1.96E−8 |
| 8 | 1.51E−8 |
| 9 | 1.22E−8 |
| 10 | 2.62E−8 |
| 11 | 8.90E−9 |
| 12 | 1.57E−8 |
| 13 | 1.34E−8 |
| 14 | 2.15E−8 |
| 15 | 7.76E−9 |
| 16 | 5.71E−9 |
| 17 | 7.95E−9 |
| 18 | 4.54E−8 |
| 19 | 3.76E−8 |
| 20 | 6.60E−10 |
| 21 | 8.40E−8 |
| 22 | 6.56E−8 |
| 23 | 1.05E−7 |
| 24 | 5.97E−8 |
| 25 | 1.52E−7 |
| 26 | 3.81E−8 |
| 27 | 5.95E−8 |
| 28 | 1.38E−7 |
| 29 | 1.22E−7 |
| 30 | 6.86E−8 |
| 31 | 7.15E−8 |
| 32 | 1.05E−8 |
| 33 | 2.50E−8 |
| Reference | 2.79E−7 |
| Example 1 | |

1. A compound of formula (I): 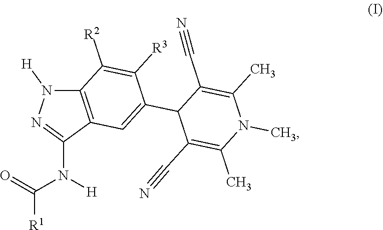
wherein
R¹ is a group selected from the group consisting of 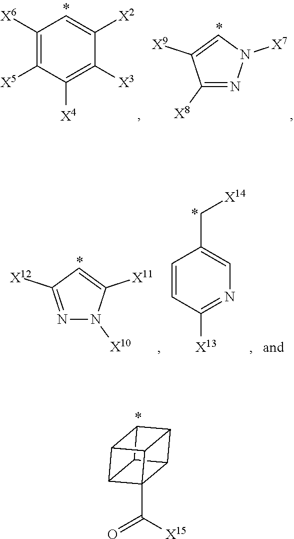
wherein “*” is the point of attachment to the rest of the molecule,
and
wherein X² is a hydrogen atom or a halogen atom or a group selected from the group consisting of
C₁-C₂-alkyl and C₁-C₂-haloalkyl,
wherein X³ is a hydrogen atom,
wherein X⁴ is a hydrogen atom or a (R⁴)(R⁵)N—(C₂-C₃-alkoxy)- group,
wherein X⁵ and X⁶, independently of each other, are a hydrogen atom or a halogen atom,
wherein X⁷ is a group selected from the group consisting of C₁-C₄-alkyl, C₂-C₄-hydroxyalkyl, methoxy-(C₂-C₄-alkyl)-; and (R⁴)(R⁵)N—(C₂-C₃-alkoxy)-,
wherein X⁸ and X⁹, independently of each other, are a hydrogen atom or a halogen atom or a group selected from the group consisting of methyl and C₁-haloalkyl,
wherein X¹⁰ is a group selected from the group consisting of C₁-C₄-alkyl, C₂-C₄-hydroxyalkyl, methoxy-(C₂-C₄-alkyl)-, and (R⁴)(R⁵)N—(C₂-C₃-alkoxy)-,
wherein X¹¹ and X¹², independently of each other, are a hydrogen atom or a halogen atom or a group selected from the group consisting of methyl and C₁-haloalkyl,
wherein X¹³ and X¹⁴, independently of each other, are a hydrogen atom or a methyl group,
and
wherein X¹⁵ is a group selected from the group consisting of methoxy and —N(R⁴)(R⁵),
R² is a halogen atom or a group selected from the group consisting of C₁-C₂-alkyl, C₁-C₂-fluoroalkyl, and vinyl,
R³ is a fluorine atom or a chlorine atom,
R⁴ and R⁵ are, independently of each other, a hydrogen atom or a methyl group,
or
R⁴ and R⁵ together with the nitrogen to which they are attached are a
nitrogen containing 4- to 6-membered heterocycloalkyl group,
wherein said 4- to 6-membered nitrogen containing heterocycloalkyl group is optionally substituted, one or two times, with a methyl group,
or a stereoisomer, a tautomer, an N-oxide, a hydrate, a solvate, or a salt thereof, or a mixture of the foregoing.

2. The compound according to claim 1, wherein:
R¹ is a group selected from the group consisting of 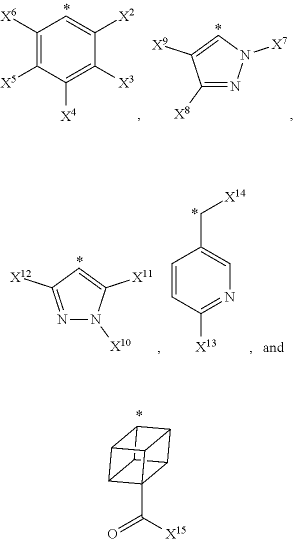
wherein “*” is the point of attachment to the rest of the molecule,
and
wherein X² is a hydrogen atom or a fluorine atom or a chlorine atom or a group selected from the group consisting of ethyl and trifluoromethyl,
wherein X³ is a hydrogen atom,
wherein X⁴ is a hydrogen atom or a 2-pyrrolidin-1-ylethoxy- group,
wherein X⁵ and X⁶, independently of each other, are a hydrogen atom or a fluorine atom,
wherein X⁷ is a group selected from the group consisting of methyl, ethyl, and isopropyl,
wherein X⁸ and X⁹, independently of each other, are a hydrogen atom or a chlorine atom or a methyl group,
wherein X¹⁰ is a group selected from the group consisting of methyl, ethyl, 2-hydroxy-2-methylpropyl, 2-methoxyethyl, and 2-pyrrolidin-1-ylethoxy,
wherein X¹¹ and X¹², independently of each other, are a hydrogen atom or a group selected from the group consisting of methyl and trifluoromethyl,
wherein X¹³ and X¹⁴, independently of each other, are a hydrogen atom or a methyl group,
and
wherein X¹⁵ is a group selected from the group consisting of methoxy, azetidin-1-yl, and 4-methylpiperazin-1-yl,
R² is an iodine atom or a group selected from the group consisting of methyl, ethyl, trifluoromethyl, and vinyl,
R³ is a fluorine atom,
or a stereoisomer, a tautomer, an N-oxide, a hydrate, a solvate, or a salt thereof, or a mixture of the foregoing.

3. The compound according to claim 1, wherein:
R¹ is a group selected from the group consisting of 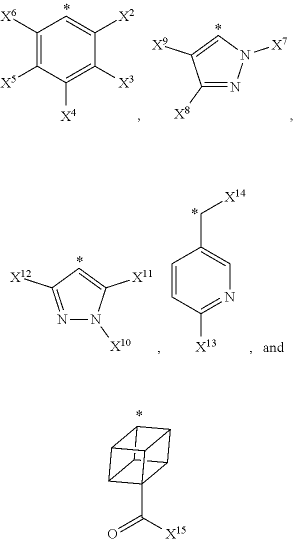
wherein “*” is the point of attachment to the rest of the molecule,
and
wherein X² is a hydrogen atom or a fluorine atom or a chlorine atom or a group selected from the group consisting of ethyl and trifluoromethyl,
wherein X³ is a hydrogen atom,
wherein X⁴ is a hydrogen atom or a 2-pyrrolidin-1-ylethoxy- group,
wherein X⁵ and X⁶, independently of each other, are a hydrogen atom or a fluorine atom,
wherein X⁷ is a group selected from the group consisting of methyl, ethyl and isopropyl,
wherein X⁸ is a hydrogen atom or a methyl group,
wherein X⁹ is a hydrogen atom or a chlorine atom,
wherein X¹⁰ is a group selected from the group consisting of methyl, ethyl, 2-hydroxy-2-methylpropyl, 2-methoxyethyl, and 2-pyrrolidin-1-ylethoxy,
wherein X¹¹ is a hydrogen atom or trifluoromethyl group,
wherein X¹² is a hydrogen atom or a group selected from the group consisting of methyl and trifluoromethyl,
wherein X¹³ and X¹⁴, independently of each other, are a hydrogen atom or a methyl group,
and
wherein X¹⁵ is a group selected from the group consisting of methoxy, azetidin-1-yl, and 4-methylpiperazin-1-yl,
R² is an iodine atom or a group selected from the group consisting of methyl, ethyl, trifluoromethyl, and vinyl,
R³ is a fluorine atom,
or a stereoisomer, a tautomer, an N-oxide, a hydrate, a solvate, or a salt thereof, or a mixture of the foregoing.

4. The compound according to claim 1, which is selected from the group consisting of:
N-[5-(3,5-dicyano-1,2,6-trimethyl-1,4-dihydropyridin-4-yl)-6-fluoro-7-iodo-1H-indazol-3-yl]-1-ethyl-3-methyl-1H-pyrazole-5-carboxamide,
2-chloro-N-[5-(3,5-dicyano-1,2,6-trimethyl-1,4-dihydropyridin-4-yl)-6-fluoro-7-iodo-1H-indazol-3-yl]benzamide,
N-[5-(3,5-dicyano-1,2,6-trimethyl-1,4-dihydropyridin-4-yl)-6-fluoro-7-iodo-1H-indazol-3-yl]-1-methyl-3-(trifluoromethyl)-1H-pyrazole-4-carboxamide,
2-chloro-N-[5-(3,5-dicyano-1,2,6-trimethyl-1,4-dihydropyridin-4-yl)-6-fluoro-7-iodo-1H-indazol-3-yl]-4-[2-(pyrrolidin-1-yl)ethoxy]benzamide,
N-[5-(3,5-dicyano-1,2,6-trimethyl-1,4-dihydropyridin-4-yl)-6-fluoro-7-methyl-1H-indazol-3-yl]-1-ethyl-3-methyl-1H-pyrazole-5-carboxamide,
2-chloro-N-[5-(3,5-dicyano-1,2,6-trimethyl-1,4-dihydropyridin-4-yl)-6-fluoro-7-methyl-1H-indazol-3-yl]benzamide,
N-[5-(3,5-dicyano-1,2,6-trimethyl-1,4-dihydropyridin-4-yl)-6-fluoro-7-methyl-1H-indazol-3-yl]-2,6-difluorobenzamide,
N-[5-(3,5-dicyano-1,2,6-trimethyl-1,4-dihydropyridin-4-yl)-6-fluoro-7-methyl-1H-indazol-3-yl]-2-ethylbenzamide,
N-[5-(3,5-dicyano-1,2,6-trimethyl-1,4-dihydropyridin-4-yl)-6-fluoro-7-methyl-1H-indazol-3-yl]-1-methyl-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-5-fluoro-2-(trifluoromethyl)benzamide,
2-chloro-N-[5-(3,5-dicyano-1,2,6-trimethyl-1,4-dihydropyridin-4-yl)-6-fluoro-7-methyl-1H-indazol-3-yl]-6-fluorobenzamide,
N-[5-(3,5-dicyano-1,2,6-trimethyl-1,4-dihydropyridin-4-yl)-6-fluoro-7-methyl-1H-indazol-3-yl]-1-ethyl-3-methyl-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-4-[2-(pyrrolidin-1-yl)ethoxy]benzamide,
N-[5-(3,5-dicyano-1,2,6-trimethyl-1,4-dihydropyridin-4-yl)-6-fluoro-7-methyl-1H-indazol-3-yl]-2-(6-methylpyridin-3-yl)acetamide,
N-[5-(3,5-dicyano-1,2,6-trimethyl-1,4-dihydropyridin-4-yl)-6-fluoro-7-methyl-1H-indazol-3-yl]-1-(2-methoxyethyl)-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-[2-(pyrrolidin-1-yl)ethyl]-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-(2-hydroxy-2-methylpropyl)-3-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-(2-hydroxy-2-methylpropyl)-5-(trifluoromethyl)-1H-pyrazole-4-carboxamide,
N-[5-(3,5-dicyano-1,2,6-trimethyl-1,4-dihydropyridin-4-yl)-6-fluoro-7-methyl-1H-indazol-3-yl]-1-[2-(pyrrolidin-1-yl)ethyl]-5-(trifluoromethyl)-1H-pyrazole-4-carboxamide,
2-chloro-N-[5-(3,5-dicyano-1,2,6-trimethyl-1,4-dihydropyridin-4-yl)-6-fluoro-7-methyl-1H-indazol-3-yl]-4-[2-(pyrrolidin-1-yl)ethoxy]benzamide,
(2R)—N-[5-(3,5-dicyano-1,2,6-trimethyl-1,4-dihydropyridin-4-yl)-6-fluoro-7-methyl-1H-indazol-3-yl]-2-(pyridin-3-yl)propanamide,
methyl 4-{[5-(3,5-dicyano-1,2,6-trimethyl-1,4-dihydropyridin-4-yl)-6-fluoro-7-methyl-1H-indazol-3-yl]carbamoyl}cubane-1-carboxylate,
4-(azetidin-1-ylcarbonyl)-N-[5-(3,5-dicyano-1,2,6-trimethyl-1,4-dihydropyridin-4-yl)-6-fluoro-7-methyl-1H-indazol-3-yl]cubane-1-carboxamide,
N-[5-(3,5-dicyano-1,2,6-trimethyl-1,4-dihydropyridin-4-yl)-6-fluoro-7-methyl-1H-indazol-3-yl]-4-[(4-methylpiperazin-1-yl)carbonyl]cubane-1-carboxamide,
N-[5-(3,5-dicyano-1,2,6-trimethyl-1,4-dihydropyridin-4-yl)-7-ethyl-6-fluoro-1H-indazol-3-yl]-2,6-difluorobenzamide,
N-[5-(3,5-dicyano-1,2,6-trimethyl-1,4-dihydropyridin-4-yl)-7-ethyl-6-fluoro-1H-indazol-3-yl]-2-ethylbenzamide,
N-[5-(3,5-dicyano-1,2,6-trimethyl-1,4-dihydropyridin-4-yl)-7-ethyl-6-fluoro-1H-indazol-3-yl]-1-(propan-2-yl)-1H-pyrazole-5-carboxamide,
4-chloro-N-[5-(3,5-dicyano-1,2,6-trimethyl-1,4-dihydropyridin-4-yl)-7-ethyl-6-fluoro-1H-indazol-3-yl]-1-methyl-1H-pyrazole-5-carboxamide,
N-[5-(3,5-dicyano-1,2,6-trimethyl-1,4-dihydropyridin-4-yl)-7-ethyl-6-fluoro-1H-indazol-3-yl]-1-ethyl-3-methyl-1H-pyrazole-5-carboxamide,
2-chloro-N-[5-(3,5-dicyano-1,2,6-trimethyl-1,4-dihydropyridin-4-yl)-7-ethyl-6-fluoro-1H-indazol-3-yl]benzamide,
2-chloro-N-[5-(3,5-dicyano-1,2,6-trimethyl-1,4-dihydropyridin-4-yl)-6-fluoro-7-(trifluoromethyl)-1H-indazol-3-yl]benzamide,
N-[5-(3,5-dicyano-1,2,6-trimethyl-1,4-dihydropyridin-4-yl)-6-fluoro-7-(trifluoromethyl)-1H-indazol-3-yl]-1-ethyl-3-methyl-1H-pyrazole-5-carboxamide, and
N-[5-(3,5-dicyano-1,2,6-trimethyl-1,4-dihydropyridin-4-yl)-7-ethenyl-6-fluoro-1H-indazol-3-yl]-1-ethyl-3-methyl-1H-pyrazole-5-carboxamide,
or a stereoisomer, a tautomer, an N-oxide, a hydrate, a solvate, or a salt thereof, or a mixture of the foregoing.

5. (canceled)

6. A pharmaceutical composition comprising the compound of formula (I) according to claim 1, or a stereoisomer, a tautomer, an N-oxide, a hydrate, a solvate, or a salt thereof, or a mixture of the foregoing, and one or more pharmaceutically acceptable excipients.

7. A pharmaceutical combination comprising:
one or more compounds of formula (I) according to claim 1, or a stereoisomer, a tautomer, an N-oxide, a hydrate, a solvate, or a salt thereof, or a mixture of the foregoing, and
one or more further active ingredients.

8. A method for treatment or prophylaxis of a disease, comprising administering to a mammal in need thereof an effective amount of a compound of formula (I) according to claim 1, or a stereoisomer, a tautomer, an N-oxide, a hydrate, a solvate, or a salt thereof, or a mixture of the foregoing.

9. (canceled)

10. The method according to claim 8 wherein the disease is a hyperproliferative disorder.

11. The method according to claim 10, wherein the disease is a cancer.

12. The method according to claim 11, wherein the cancer disease is selected from the group consisting of breast cancer, cancer of the respiratory tract, brain cancer, prostate cancer, testicular cancer, endometrial cancer, cervical cancer, ovarian cancer, vaginal cancer, vulvar cancer, sarcoma of the uterus, anal cancer, colon cancer, colorectal cancer, oesophageal cancer, gallbladder cancer, gastric cancer, pancreatic cancer, rectal cancer, small-intestine cancer, salivary gland cancer, bladder cancer, penile cancer, kidney cancer, renal pelvis cancer, ureter cancer, urethral cancer, human papillary renal cancer, eye cancer, liver cancer, skin cancer, head-and-neck cancer, lymphoma, sarcoma and leukemia.

13. The pharmaceutical combination according to claim 7, wherein the one or more further active ingredients are anti-cancer agents.
